(19) United States
(12) Patent Application Publication
(10) Pub. No.: US 2025/0339526 A1
(43) Pub. Date: Nov. 6, 2025

(54) METHODS FOR TREATING CANCER WITH ENHANCED ANTIGEN PRESENTING CELLS

(71) Applicant: STEMCELL TECHNOLOGIES CANADA INC., Vancouver (CA)

(72) Inventors: Ricardo Zwirtes, Watertown, MA (US); Oliver Rosen, Watertown, MA (US); Andrew Elnatan, Watertown, MA (US)

(21) Appl. No.: 18/997,693

(22) PCT Filed: Jul. 28, 2023
(86) PCT No.: PCT/US2023/071265

§ 371 Date: Jan. 22, 2025

Related U.S. Application Data
(60) Provisional application No. 63/369,752, filed on Jul. 28, 2022.

(51) Int. Cl. A61K 40/11; A61K 35/15; A61K 35/17; A61K 40/13; A61K 40/15; A61K 40/17; A61K 40/46; A61K 45/06; A61P 35/04; C12N 5/0781; C12N 5/0783; C12N 5/0786

(52) U.S. Cl. CPC A61K 40/11; A61K 35/15; A61K 35/17; A61K 40/13; A61K 40/15; A61K 40/17; A61K 40/46; A61K 45/06; A61P 35/04; C12N 5/0635; C12N 5/0636; C12N 5/0645; C12N 5/0646

(57) ABSTRACT

The present application provides enhanced APCs for treating HPV-associated cancers. The enhanced APCs are capable of activating T cells in an HLA agnostic manner and are derived from input APCs in which at least one nucleic acid encoding an HPV antigen has been delivered intracellularly. In some aspects, the enhanced APCs are administered in combination with an immune checkpoint inhibitor, such as an antagonist of PD-1/PD-L1 and/or an antagonist of CTLA-4.

### CROSS-REFERENCE TO RELATED APPLICATIONS

This PCT application claims the priority benefit of U.S. Provisional Application No. 63/369,752, filed Jul. 28, 2022, which is herein incorporated by reference in its entirety.

### REFERENCE TO SEQUENCE LISTING SUBMITTED ELECTRONICALLY

The content of the sequence listing is submitted electronically (Name: 4821_088PC01_SequenceListing_ST26.XML; Size: 35,417 bytes; and Date of Creation: Jul. 28, 2023) and is filed with the application herein incorporated by reference in its entirety.

### FIELD OF THE DISCLOSURE

The present disclosure generally relates to methods of using modified antigen presenting cells (“enhanced APCs”) that are capable of activating T cells in an HLA-agnostic manner to treat HPV-associated cancers in a subject, as well as doses and regimens thereof.

### BACKGROUND

Human papillomavirus, or HPV, is a virus that infects many people. In fact, more than 75% of women and men will be infected at some point in life. While most HPV infections are subclinical and will cause no physical symptoms, in some people infections can cause growths known as papillomas, and can even cause cancers of the cervix, vulva, vagina, penis, oropharynx and anus. In particular, HPV16 and HPV18 are known to cause around 70% of cervical cancer cases. There are currently no treatments available for HPV itself. Current treatment options generally aim to treat the various ailments that can be associated with HPV infection. While vaccine (e.g., GARDASIL®) has been approved, it is strictly preventive in nature and not without side effects. Therefore, new and alternative treatment options for HPV infection is needed.

All references cited herein, including patent applications and publications, are incorporated by reference in their entirety. The patent publications WO 2013/059343, WO 2015/023982, WO 2016/070136, WO2017041050, WO2017008063, WO 2017/192785, WO 2017/192786, WO 2019/178005, WO 2019/178006, WO 2020/072833, WO 2020/154696, and WO 2020/176789, US20180142198, and U.S. Pat. No. 20,180,201889 are hereby expressly incorporated by reference in their entirety.

### BRIEF SUMMARY OF THE DISCLOSURE

Provided herein is a method for treating a human papilloma virus (HPV)-associated cancer in a subject in need thereof, the method comprising administering to the subject a composition comprising modified antigen presenting cells (“enhanced APCs”), wherein the enhanced APCs comprise an HPV antigen and are capable of activating T cells in an HLA agnostic manner. In some aspects, the method further comprises administering to the subject an immune checkpoint inhibitor. Also provided herein is a method for treating a human papilloma virus (HPV)-associated cancer in a subject in need thereof, the method comprising administering to the subject (i) a composition comprising modified antigen presenting cells (“enhanced APCs”), wherein the enhanced APCs comprise an HPV antigen and are capable of activating T cells in an HLA agnostic manner, and (ii) an immune checkpoint inhibitor.

In some aspects, the enhanced APCs comprise T cells, B cells, NK cells, monocytes, or combinations thereof. In some aspects, the HPV antigen comprises an HPV-16 antigen or an HPV-18 antigen. In some aspects, the HPV antigen comprises a peptide derived from HPV E6 and/or HPV E7. In some aspects, the HPV antigen comprises a peptide derived from HPV E6 and a peptide derived from HPV E7. In some aspects, the peptide derived from HPV E6 and/or HPV E7 is full-length E6 and/or E7. In some aspects, the HPV antigen comprises the amino acid sequence set forth in any one of SEQ ID NOs: 14-17. In some aspects, the HPV antigen comprises a first antigen comprising the amino acid sequence set forth in SEQ ID NO: 14 and a second antigen comprising the amino acid sequence set forth in SEQ ID NO: 15. In some aspects, the HPV antigen comprises a first antigen comprising the amino acid sequence set forth in SEQ ID NO: 16 and a second antigen comprising the amino acid sequence set forth in SEQ ID NO: 17.

In some aspects, the enhanced APCs exhibit increased expression of a co-stimulatory molecule as compared to corresponding non-modified APCs (“reference APCs”). In some aspects, the co-stimulatory molecule comprises CD86. In some aspects, the enhanced APCs exhibit increased expression of a cytokine as compared to corresponding non-modified APCs (“reference APCs”). In some aspects, the cytokine comprises a membrane-bound cytokine. In some aspects, the cytokine comprises IL-2, IL-12, or both.

In some aspects, the enhanced APCs have been prepared by passing a cell suspension comprising input APCs through a cell-deforming constriction, thereby causing perturbations in the APCs such that a nucleic acid encoding the antigen, a nucleic acid encoding the co-stimulatory molecule and/or a nucleic acid encoding the cytokine enters the APCs through the perturbations when contacted with the APCs, and thereby producing the enhanced APCs. In some aspects, the cell suspension comprising the input APCs have been cultured in combination with the nucleic acid encoding the antigen, the nucleic acid encoding the co-stimulatory molecule, and/or the nucleic acid encoding the cytokine, such that the nucleic acid encoding the antigen, the nucleic acid encoding the co-stimulatory molecule, and/or the nucleic acid encoding the cytokine are in contact with the input APCs. In some aspects, the nucleic acid encoding the antigen, the nucleic acid encoding the co-stimulatory molecule, and/or the nucleic acid encoding the cytokine is an mRNA. In some aspects, the cell-deforming constriction comprises a diameter, which is about 4.2 μm to about 6 μm or about 4.2 μm to about 4.8 μm.

In any of the methods provided herein, in some aspects, the enhanced APCs have been conditioned in a medium comprising an adjuvant. In some aspects, the enhanced APCs have been conditioned in a medium comprising an adjuvant for about 2 hours to about 10 hours. In some aspects, the enhanced APCs have been conditioned in a medium comprising an adjuvant for about 3 hours to about 6 hours. In some aspects, the enhanced APCs have been conditioned in a medium comprising an adjuvant for about 4 hours. In some aspects, the enhanced APCs have been conditioned in a medium comprising an adjuvant at about 37° C. In some aspects, the adjuvant comprises a CpG oligodeoxynucleotide (ODN), LPS, IFN-α, STING agonists, RIG-I agonists, poly I:C, R837, R848, a TLR3 agonist, a TLR4 agonist, or a TLR9 agonist. In some aspects, the adjuvant is ODN.

For any of the methods provided herein comprising administering an immune checkpoint inhibitor, in some aspects, the immune checkpoint inhibitor comprises an antagonist of CTLA-4 and/or an antagonist of PD-1/PD-L1. In some aspects, the immune checkpoint inhibitor is an antagonist of PD-1/PD-L1. In some aspects, the antagonist of PD-1/PD-L1 is an antibody that binds PD-1 or an antibody that binds PD-L1. In some aspects, the antagonist of CTLA-4 is an antibody that binds CTLA-4. In some aspects, the antibody that binds PD-1 is pembrolizumab. In some aspects, the antibody that binds PD-1 is nivolumab. In some aspects, the antibody that binds PD-L1 is atezolizumab. In some aspects, the antibody that binds CTLA-4 is ipilimumab.

Provided herein is a method for treating a HPV″ recurrent, locally advanced, or metastatic tumor in a subject in need thereof, the method comprising administering to the subject a composition comprising modified antigen presenting cells (“enhanced APCs”), wherein the enhanced APCs comprise an HPV antigen and are capable of activating T cells in an HLA agnostic manner.

For such a method, in some aspects, the enhanced APCs comprise T cells, B cells, NK cells, monocytes, or combinations thereof. In some aspects, the HPV antigen comprises an HPV-16 antigen or an HPV-18 antigen. In some aspects, the HPV antigen comprises a peptide derived from HPV E6 and/or HPV E7. In some aspects, the HPV antigen comprises a peptide derived from HPV E6 and a peptide derived from HPV E7. In some aspects, the peptide derived from HPV E6 and/or HPV E7 is full-length E6 and/or E7. In some aspects, the HPV antigen comprises the amino acid sequence set forth in any one of SEQ ID NOs: 14-17. In some aspects, the HPV antigen comprises a first antigen comprising the amino acid sequence set forth in SEQ ID NO: 14 and a second antigen comprising the amino acid sequence set forth in SEQ ID NO: 15. In some aspects, the HPV antigen comprises a first antigen comprising the amino acid sequence set forth in SEQ ID NO: 16 and a second antigen comprising the amino acid sequence set forth in SEQ ID NO: 17.

In some aspects, the enhanced APCs exhibit increased expression of a co-stimulatory molecule as compared to corresponding non-modified APCs (“reference APCs”). In some aspects, the co-stimulatory molecule comprises CD86. In some aspects, the enhanced APCs exhibit increased expression of a cytokine as compared to corresponding non-modified APCs (“reference APCs”). In some aspects, the cytokine comprises a membrane-bound cytokine. In some aspects, the cytokine comprises IL-2, IL-12, or both.

In some aspects, the enhanced APCs have been prepared by passing a cell suspension comprising input APCs through a cell-deforming constriction, thereby causing perturbations in the APCs such that a nucleic acid encoding the antigen, a nucleic acid encoding the co-stimulatory molecule and/or a nucleic acid encoding the cytokine enters the APCs through the perturbations when contacted with the APCs, and thereby producing the enhanced APCs. In some aspects, the cell suspension comprising the input APCs have been cultured in combination with the nucleic acid encoding the antigen, the nucleic acid encoding the co-stimulatory molecule, and/or the nucleic acid encoding the cytokine, such that the nucleic acid encoding the antigen, the nucleic acid encoding the co-stimulatory molecule, and/or the nucleic acid encoding the cytokine are in contact with the input APCs.

In some aspects, the nucleic acid encoding the antigen, the nucleic acid encoding the co-stimulatory molecule, and/or the nucleic acid encoding the cytokine is an mRNA. In some aspects, the cell-deforming constriction comprises a diameter, which is about 4.2 μm to about 6 μm or about 4.2 μm to about 4.8 μm.

In some aspects, the enhanced APCs have been conditioned in a medium comprising an adjuvant. In some aspects, the enhanced APCs have been conditioned in a medium comprising an adjuvant for about 2 hours to about 10 hours. In some aspects, the enhanced APCs have been conditioned in a medium comprising an adjuvant for about 3 hours to about 6 hours. In some aspects, the enhanced APCs have been conditioned in a medium comprising an adjuvant for about 4 hours. In some aspects, the enhanced APCs have been conditioned in a medium comprising an adjuvant at about 37° C. In some aspects, the adjuvant comprises a CpG oligodeoxynucleotide (ODN), LPS, IFN-α, STING agonists, RIG-I agonists, poly I:C, R837, R848, a TLR3 agonist, a TLR4 agonist, or a TLR9 agonist. In some aspects, the adjuvant is ODN.

For any of the above methods, in some aspects, the composition comprising enhanced APCs is administered in combination with an immune checkpoint inhibitor. In some aspects, the one or more immune checkpoint inhibitors comprises an antagonist of CTLA-4 and/or an antagonist of PD-1/PD-L1. In some aspects, the immune checkpoint inhibitor is an antagonist of PD-1/PD-L1. In some aspects, the antagonist of PD-1/PD-L1 is an antibody that binds PD-1 or an antibody that binds PD-L1. In some aspects, the antagonist of CTLA-4 is an antibody that binds CTLA-4. In some aspects, the antibody that binds PD-1 is pembrolizumab. In some aspects, the antibody that binds PD-1 is nivolumab. In some aspects, the antibody that binds PD-L1 is atezolizumab. In some aspects, the antibody that binds CTLA-4 is ipilimumab.

In some aspects, the subject that can be treated using the methods provided herein is a human. In some aspects, the subject is positive for human papillomavirus type 16 (HPV16″). In some aspects, the HPV-associated cancer and/or HPV″ tumor comprises a cervical cancer, anal cancer, vulval cancer, vaginal cancer, penile cancer, or oropharyngeal cancer.

For any of the treating methods provided herein, in some aspects, the composition comprising enhanced APCs is administered in an amount of about 0.25×10⁶ cells/kg to about 7.50×10⁶ cells/kg. In some aspects, the composition comprising enhanced APCs is administered in an amount of about 0.5×10⁶ cells/kg to about 5.0×10⁶ cells/kg. In some aspects, the composition comprising enhanced APCs is administered in an amount of about 0.5×10⁶ cells/kg. In some aspects, the composition comprising enhanced APCs is administered in an amount of about 2.5×10⁶ cells/kg. In some aspects, the composition comprising enhanced APCs is administered in an amount of about 5.0×10⁶ cells/kg. In some aspects, the composition comprising enhanced APCs is administered in an amount of about 0.25×10⁶ cells/kg. In some aspects, the composition comprising enhanced APCs is administered in an amount of about 1.25×10⁶ cells/kg. In some aspects, the composition comprising enhanced APCs is administered in an amount of about 3.25×10⁶ cells/kg. In some aspects, the composition comprising enhanced APCs is administered in an amount of about 7.5×10⁶ cells/kg.

For any of the treating methods provided herein, in some aspects, the composition comprising enhanced APCs is administered intravenously. Where the method comprises administering an immune checkpoint inhibitor, in some aspects, the immune checkpoint inhibitor is administered intravenously, orally, or subcutaneously. In some aspects, the immune checkpoint inhibitor is administered intravenously.

In some aspects, pembrolizumab is administered in an amount of about 10 mg to about 400 mg. In some aspects, pembrolizumab is administered in an amount of about 200 mg to about 400 mg. In some aspects, pembrolizumab is administered in an amount of about 200 mg.

In some aspects, nivolumab is administered in an amount of about 15 mg to about 500 mg. In some aspects, nivolumab is administered in an amount of about 240 mg to about 480 mg. In some aspects, nivolumab is administered in an amount of about 360 mg.

In some aspects, atezolizumab is administered in an amount of about 75 mg to about 1700 mg. In some aspects, atezolizumab is administered in an amount of about 840 mg to about 1680 mg. In some aspects, atezolizumab is administered in an amount of about 1200 mg.

In some aspects, ipilimumab is administered in an amount of about 1 mg/kg to about 10 mg/kg. In some aspects, ipilimumab is administered in an amount of about 1 mg/kg to about 3 mg/kg.

For any of the treatment methods provided herein, in some aspects, the composition comprising enhanced APCs is administered on day 1 of a three-week cycle. In some aspects, the composition comprising enhanced APCs is further administered on day 2 of a first three-week cycle. In some aspects, about 0.25×10⁶ cells/kg, about 0.5×10⁶ cells/kg, about 1.25×10⁶ cells/kg, about 2.5×10⁶ cells/kg, about 3.25×10⁶ cells/kg, about 5.0×10⁶ cells/kg, or about 7.50×10⁶ cells/kg are administered on day 1 of each three-week cycle. In some aspects, about 0.25×10⁶ cells/kg, about 0.5×10⁶ cells/kg, about 1.25×10⁶ cells/kg, about 2.5×10⁶ cells/kg, about 3.25×10⁶ cells/kg, about 5.0×10⁶ cells/kg, or about 7.50×10⁶ cells/kg are administered on day 2 of the first three-week cycle.

In some aspects, the antibody that binds PD-1, the antibody that binds PD-L1, and/or the antibody that binds CTLA-4 is administered once per three-week cycle. In some aspects, the antibody that binds PD-1 is administered on day 1 of a three-week cycle. In some aspects, the antibody that binds PD-1 is administered on day 8 of the first three-week cycle. In some aspects, the antibody that binds PD-1 is administered on day 8 of the first three-week cycle and day 1 of each subsequent three-week cycle. In some aspects, the antibody that binds PD-1 is administered on day 1 of the third three-week cycle. In some aspects, the antibody that binds PD-1 is administered on day 1 of the third three-week cycle and day 1 of each subsequent three-week cycle. In some aspects, the antibody that binds PD-1 is pembrolizumab, wherein the pembrolizumab is administered in an amount of about 200 mg. In some aspects, the antibody that binds PD-1 is nivolumab, wherein the nivolumab is administered in an amount of about 360 mg. In some aspects, the antibody that binds CTLA-4 is administered on day 1 of each three-week cycle. In some aspects, the antibody that binds CTLA-4 is administered once per two three-week cycles. In some aspects, the antibody that binds CTLA-4 is ipilimumab, wherein the ipilimumab is administered at a dose of about 3 mg/kg. In some aspects, the antibody that binds PD-L1 is administered on day 8 of the first three-week cycle and day 1 of each subsequent cycle. In some aspects, the antibody that binds PD-L1 is atezolizumab, wherein the atezolizumab is administered at a dose of about 1200 mg.

For any of the treating methods provided herein, in some aspects, the composition comprising enhanced APCs is administered to the subject for at least about 3 months, 6 months, 9 months, 12 months, 15 months, 18 months, 21 months, or 24 months.

In some aspects, the composition comprising enhanced APCs comprises: (a) about 5×10⁶ enhanced APCs to about 1×10⁹ enhanced APCs, (b) cryopreservation medium at a concentration of about 40% to about 95% (w/w), (c) hypothermic preservation medium at a concentration of about 25% to about 35% (w/w), and (d) human serum albumin solution at a concentration of 15% to about 25% (w/w), wherein the pH of the composition is about pH 6.0 to about pH 8.5. In some aspects, the composition comprising enhanced APCs comprises: (a) about 8.1×10⁷ enhanced APCs, (b) cryopreservation medium at a concentration of about 50% (w/w), (c) hypothermic preservation medium at a concentration of about 30% (w/w), and (d) human serum albumin solution at a concentration of 20% (w/w), wherein the pH of the composition is about pH 7.0 to about pH 7.9. In some aspects, the composition comprising enhanced APCs comprises: (a) about 1.05×10⁸ enhanced APCs, (b) cryopreservation medium at a concentration of about 50% (w/w), (c) hypothermic preservation medium at a concentration of about 30% (w/w), and (d) human serum albumin solution at a concentration of 20% (w/w), wherein the pH of the composition is about pH 7.0 to about pH 7.9.

For any of the methods provided herein, in some aspects, the composition comprising enhanced APCs comprises about 1×10⁶ enhanced APCs/mL to about 1×10⁸ enhanced APCs/mL. In some aspects, the composition comprising enhanced APCs comprises about 8.5×10⁶ enhanced APCs/mL. In some aspects, the composition comprising enhanced APCs comprises about 1.1×10⁷ enhanced APCs/mL.

In some aspects, the cryopreservation medium is CryoStor® CS10. In some aspects, the hypothermic preservation medium is HypoThermasol® FRS.

Provided herein is a method for treating a human papilloma virus (HPV)-associated cancer in a subject in need thereof, the method comprising: administering to the subject: (i) a composition comprising modified antigen presenting cells (“enhanced APCs”), wherein the enhanced APCs comprise an HPV E6 and/or HPV E7 antigen, CD86, membrane-bound interleukin-2 (“mbIL-2”), and membrane-bound interleukin-12 (“mbIL-12”), wherein the enhanced APCs are capable of activating T cells in an HLA agnostic manner, and (ii) an antagonist of PD-1. Also provided herein is a method for for treating a HPV″ recurrent, locally advanced, or metastatic tumor in a subject in need thereof, the method comprising administering a composition comprising modified antigen presenting cells (“enhanced APCs”), wherein the enhanced APCs comprise an HPV E6 and/or HPV E7 antigen, CD86, membrane-bound interleukin-2 (“mbIL-2”), and membrane-bound interleukin-12 (“mbIL-12”), and wherein the enhanced APCs are capable of activating T cells in an HLA agnostic manner.

In some aspects, the composition comprising enhanced APCs is administered in combination with an antagonist of PD-1. In some aspects, the antagonist of PD-1 is an antibody that binds PD-1. In some aspects, the antibody that binds PD-1 is pembrolizumab.

In some aspects, the enhanced APCs have been prepared by passing a cell suspension comprising input APCs through a cell-deforming constriction, thereby causing perturbations in the APCs such that a nucleic acid encoding the antigen, a nucleic acid encoding the co-stimulatory moleculem and a nucleic acid encoding the cytokine enters the APCs through the perturbations when contacted with the APCs, and thereby producing the enhanced APCs. In some aspects, the cell suspension comprising the input APCs have been cultured in combination with the nucleic acid encoding the antigen, the nucleic acid encoding the co-stimulatory molecule, and the nucleic acid encoding the cytokine, such that the nucleic acid encoding the antigen, the nucleic acid encoding the co-stimulatory molecule, and the nucleic acid encoding the cytokine are in contact with the input APCs.

In some aspects, the nucleic acid encoding the antigen, the nucleic acid encoding the co-stimulatory molecule, and the nucleic acid encoding the cytokine is an mRNA. In some aspects, the cell-deforming constriction comprises a diameter, which is about 4.2 μm to about 6 μm or about 4.2 μm to about 4.8 μm.

In some aspects, the enhanced APCs have been conditioned in a medium comprising an adjuvant. In some aspects, the enhanced APCs have been conditioned in a medium comprising an adjuvant for about 2 hours to about 10 hours. In some aspects, the enhanced APCs have been conditioned in a medium comprising an adjuvant for about 3 hours to about 6 hours. In some aspects, the enhanced APCs have been conditioned in a medium comprising an adjuvant for about 4 hours. In some aspects, the enhanced APCs have been conditioned in a medium comprising an adjuvant at about 37° C. In some aspects, the adjuvant comprises a CpG oligodeoxynucleotide (ODN), LPS, IFN-α, STING agonists, RIG-I agonists, poly I:C, R837, R848, a TLR3 agonist, a TLR4 agonist, or a TLR9 agonist. In some aspects, the adjuvant is ODN.

### BRIEF DESCRIPTION OF THE DRAWINGS/FIGURES

FIG. 1 shows a modified APC with intracellular HPV-16 E6 mRNA, HPV-16 E7 mRNA, CD86 mRNA, mbIL-2 mRNA, and mbIL-12 mRNA. The modified APC presents on its cell surface various types of HLA molecules, HPV-16 E6 and HPV-16 E7 epitopes, CD86, mbIL-2, and mbIL-12.

FIG. 2 shows a schematic of an exemplary mechanism of action of enhanced APCs described herein, which are created from peripheral blood mononuclear cells (PBMCs) squeeze processed with mRNAs encoding for one or more HPV antigens, co-stimulatory molecules, and/or membrane-bound cytokines. The enhanced APCs migrate to lymphoid organs, where the presentation of E6 and E7 epitopes stimulate E6-specific and E7-specific T cells that are capable of generating a specific antitumor response targeting HPV16⁺ cells while minimizing the risk of accidently attacking healthy tissues.

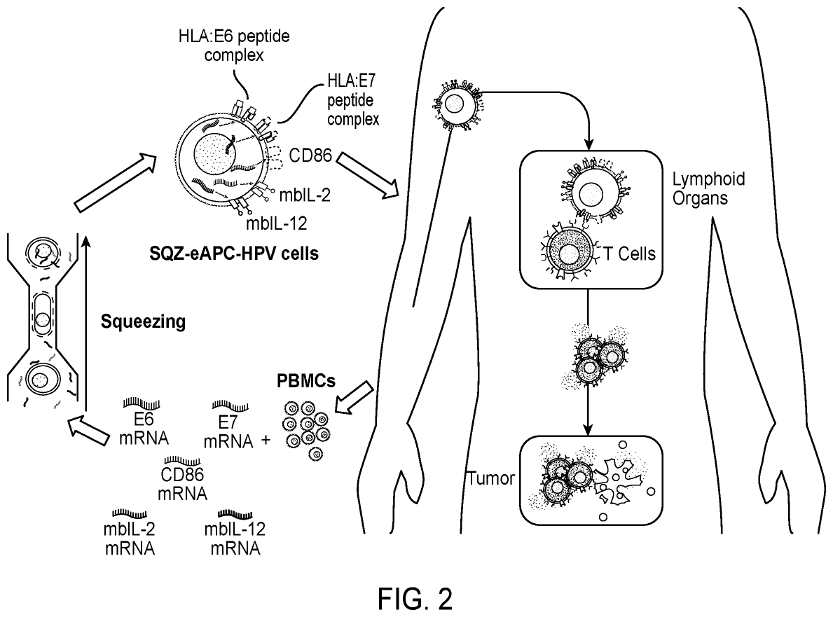

FIG. 3 shows anti-tumor immune response after treatment with enhanced APCs. The top panels (i.e., bar graphs) show percentile increase or reduction of expression of CD8, HLA-1, E6, and PD-L1 in tumor cells at date of screening and 28 days post-treatment with enhanced APCs. The bottom panels show corresponding immunohistochemical staining of CD8, HLA-1, E6, and PD-L1 in tumor biopsies at date of screening and 28 days post-treatment with enhanced APCs.

FIG. 4 shows a schematic of a clinical study design of a monotherapy of enhanced APCs administered at the indicated dose every three weeks (q3w) during a monotherapy phase, followed by a combination phase involving administration of the enhanced APCs in combination with 200 mg of Pembrolizumab every three weeks (q3w); and of a lead-in phase involving administration of enhanced APCs as a monotherapy for 6 weeks, followed by combination therapy of the enhanced APCs with 200 mg of Pembrolizumab every three weeks (q3w).

FIG. 5 shows a monotherapy dosing schematic of enhanced APCs administered on Day 1 of each cycle.

FIG. 6 shows staggered enrollment of the first two patients in each cohort of a clinical study design for a therapy involving administration of enhanced APCs.

FIG. 7 shows the safety schematic of a clinical study design for a combination therapy involving administration of enhanced APCs with Pembrolizumab.

FIG. 8 shows a schematic of a clinical study design of a combination therapy involving administration of enhanced APCs with Pembrolizumab.

FIG. 9 shows a schematic of dose administration modifications in response to adverse events related to the infusion of enhanced APCs as part of a safety assessment for a clinical study of a therapy involving administration of enhanced APCs.

### DETAILED DESCRIPTION OF THE DISCLOSURE

The present application generally relates to the use of enhanced APCs to treat a human papilloma virus (HPV)-associated cancer in a subject in need thereof. The enhanced APCs have been modified such that the APCs exhibit one or more enhanced properties. Non-limiting examples of such enhanced properties are provided throughout the present disclosure. Unless indicated otherwise, the terms “enhanced APCs” (or derivatives thereof) and “modified APCs”) (or derivatives thereof) are used interchangeably to described such APCs. As further described herein, the enhanced APCs differ from other APCs in that the cells are capable of activating T cells in a HLA-agnostic manner. Accordingly, the methods provided herein are useful in various clinical settings and allow for the treatment of diverse subjects irrespective of HLA haplotype. Additional aspects of the present disclosure are provided further below.

### General Techniques

The techniques and procedures described or referenced herein are generally well understood and commonly employed using conventional methodology by those skilled in the art, such as, for example, the widely utilized methodologies described in Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F.M. Ausubel, et al. eds., 2003); the series Methods in Enzymology (Academic Press, Inc.); PCR 2: A Practical Approach (M.J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6^{th} ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 2011).

### Definitions

Unless defined otherwise, all technical and scientific terms used herein have the same meaning as commonly understood by one of ordinary skill in the art to which this disclosure belongs. In case of conflict, the present application including the definitions shall control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural references unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of aspects of “consisting essentially of” or “consisting of.”

The term “about” as used herein refers to the usual error range for the respective value readily known to the skilled person in this technical field, and is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower). Furthermore, reference to “about” as a value or parameter herein includes and describes aspects that are directed to that value or parameter per se. For example, a description referring to “about X” includes “X” as described.

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

The term “effective amount” or “pharmaceutically effective amount” or “therapeutically effective amount” as used herein refers to the amount or quantity of a drug or pharmaceutically active substance which is sufficient to elicit the required or desired therapeutic response, or in other words, the amount which is sufficient to elicit an appreciable biological response when administered to a patient.

As used herein, the term “HLA-agnostic manner” means independent of human leukocyte antigen (HLA) haplotype. Generally, T cell activation requires the recognition of antigens (as peptide fragments) presented on “Human leukocyte antigen” or “HLA,” which are expressed on certain cells. Because of the variability that exists in HLA molecules, certain peptides are restricted or binds only to certain HLA molecules. Therefore, whether a particular antigenic peptide fragment induces an immune response in a subject closely depends on the particular HLA molecules that are present within the subject. As is apparent from the present disclosure, because the enhanced APCs described herein are capable of activating T cells in a HLA-agnostic manner, they can have therapeutic effects in a much larger population.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this disclosure. Allelic variation or naturally occurring mutational events do not give rise to heterologous DNA, as used herein.

The term “heterologous” as it relates to amino acid sequences such as peptide sequences and polypeptide sequences, denotes sequences that are not normally joined together, and/or are not normally associated with a particular cell. Thus, a “heterologous” region of a peptide sequence is a segment of amino acids within or attached to another amino acid molecule that is not found in association with the other molecule in nature. For example, a heterologous region of a peptide construct could include the amino acid sequence of the peptide flanked by sequences not found in association with the amino acid sequence of the peptide in nature. Another example of a heterologous peptide sequence is a construct where the peptide sequence itself is not found in nature (e.g., synthetic sequences having amino acids different as coded from the native gene). Similarly, a cell transformed with a vector that expresses an amino acid construct which is not normally present in the cell would be considered heterologous for purposes of this disclosure. Allelic variation or naturally occurring mutational events do not give rise to heterologous peptides, as used herein.

The term “exogenous” when used in reference to an agent, such as an antigen or an adjuvant, with relation to a cell refers to an agent outside of the cell or an agent delivered into the cell from outside the cell. The cell can or can not have the agent already present, and can or can not produce the agent after the exogenous agent has been delivered.

The term “homologous” as used herein refers to a molecule which is derived from the same organism. In some aspects, the term refers to a nucleic acid or protein which is normally found or expressed within the given organism.

The term “autologous” as used herein refers to biological material (e.g., cells or tissue) obtained from the same subject. In some aspects, “autologous” cells or tissue of a subject are put back into the same subject.

As used herein, “treatment” or “treating” is an approach for obtaining beneficial or desired results, including clinical results. For purposes of this disclosure, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (e.g., tumor volume). The methods of the disclosure contemplate any one or more of these aspects of treatment. In the context of cancer, “treating” includes any or all of killing cancer cells, inhibiting growth of cancer cells, inhibiting replication of cancer cells, lessening of overall tumor burden and ameliorating one or more symptoms associated with the disease.

As used herein, the term “prophylactic treatment” refers to treatment, wherein an individual is known or suspected to have or be at risk for having a disorder but has displayed no symptoms or minimal symptoms of the disorder. An individual undergoing prophylactic treatment can be treated prior to onset of symptoms. In some aspects, an individual can be treated if they have a precancerous lesion, particularly a precancerous lesion associated with HPV infection.

As used herein, the term “combination therapy” means that a first agent can be administered in conjunction with another agent. “In conjunction with” refers to administration of one treatment modality in addition to another treatment modality, such as administration of a composition of PMBCs as described herein in addition to administration of an immunoconjugate as described herein to the same individual. As such, “in conjunction with” refers to administration of one treatment modality before, during, or after delivery of the other treatment modality to the individual.

The term “simultaneous administration” as used herein means that a first therapy and second therapy in a combination therapy are administered with a time separation of no more than about 15 minutes, such as no more than about any of 10, 5, or 1 minutes. When the first and second therapies are administered simultaneously, the first and second therapies can be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy can be administered first. The first and second therapies are contained in separate compositions, which can be contained in the same or different packages or kits.

As used herein, the term “concurrent administration” means that the administration of the first therapy and that of a second therapy in a combination therapy overlap with each other.

As used herein, the term “modulate” can refer to the act of changing, altering, varying, or otherwise modifying the presence, or an activity of, a particular target. For example, modulating an immune response can refer to any act leading to changing, altering, varying, or otherwise modifying an immune response. In some aspects, “modulate” refers to enhancing the presence or activity of a particular target. In some aspects, “modulate” refers to suppressing the presence or activity of a particular target. In other aspects, modulating the expression of a nucleic acid can include, but not limited to a change in the transcription of a nucleic acid, a change in mRNA abundance (e.g., increasing mRNA transcription), a corresponding change in degradation of mRNA, a change in mRNA translation, and so forth.

As used herein, the term “inhibit” refers to the act of blocking, reducing, eliminating, or otherwise antagonizing the presence, or an activity of, a particular target. Inhibition can refer to partial inhibition or complete inhibition. In some aspects, inhibiting an immune response can refer to any act leading to a blockade, reduction, elimination, or any other antagonism of an immune response. In other aspects, inhibition of the expression of a nucleic acid can include, but not limited to reduction in transcription of a nucleic acid, reduction of mRNA abundance (e.g., silencing mRNA transcription), degradation of mRNA, inhibition of mRNA translation, gene editing and so forth. In other aspects, inhibition of the expression of a protein can include, but not be limited to, reduction in the transcription of a nucleic acid encoding the protein, reduction in the stability of mRNA encoding the protein, inhibition of translation of the protein, reduction in stability of the protein, and so forth. In another aspect, inhibit can refer to the act of slowing or stopping growth; for example, retarding or preventing the growth of a tumor cell.

As used herein, the term “suppress” can refer to the act of decreasing, reducing, prohibiting, limiting, lessening, or otherwise diminishing the presence, or an activity of, a particular target. Suppression can refer to partial suppression or complete suppression. For example, suppressing an immune response can refer to any act leading to decreasing, reducing, prohibiting, limiting, lessening, or otherwise diminishing an immune response. In some aspects, suppression of the expression of a nucleic acid can include, but not limited to reduction in transcription of a nucleic acid, reduction of mRNA abundance (e.g., silencing mRNA transcription), degradation of mRNA, inhibition of mRNA translation, and so forth. In other aspects, suppression of the expression of a protein can include, but not be limited to, reduction in the transcription of a nucleic acid encoding the protein, reduction in the stability of mRNA encoding the protein, inhibition of translation of the protein, reduction in stability of the protein, and so forth.

As used herein, the term “enhance” can refer to the act of improving, boosting, heightening, or otherwise increasing the presence, or an activity of, a particular target. For example, enhancing an immune response can refer to any act leading to improving, boosting, heightening, or otherwise increasing an immune response. In one aspect, enhancing an immune response can refer to employing an antigen and/or adjuvant to improve, boost, heighten, or otherwise increase an immune response. In some aspects, enhancing the expression of a nucleic acid can include, but not limited to increase in the transcription of a nucleic acid, increase in mRNA abundance (e.g., increasing mRNA transcription), decrease in degradation of mRNA, increase in mRNA translation, and so forth. In other aspects, enhancing the expression of a protein can include, but not be limited to, increase in the transcription of a nucleic acid encoding the protein, increase in the stability of mRNA encoding the protein, increase in translation of the protein, increase in the stability of the protein, and so forth.

As used herein, the term “induce” can refer to the act of initiating, prompting, stimulating, establishing, or otherwise producing a result. For example, inducing an immune response can refer to any act leading to initiating, prompting, stimulating, establishing, or otherwise producing a desired immune response. In some aspects, inducing the expression of a nucleic acid can include, but not limited to initiation of the transcription of a nucleic acid, initiation of mRNA translation, and so forth. In other aspects, inducing the expression of a protein can include, but not be limited to, increase in the transcription of a nucleic acid encoding the protein, increase in the stability of mRNA encoding the protein, increase in translation of the protein, increase in the stability of the protein, and so forth.

The term “polynucleotide” or “nucleic acid” as used herein refers to a polymeric form of nucleotides of any length, including ribonucleotides and deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as can typically be found in RNA or DNA), or modified or substituted sugar or phosphate groups. The backbone of the polynucleotide can comprise repeating units, such as N-(2-aminoethyl)-glycine, linked by peptide bonds (i.e., peptide nucleic acid). Alternatively, the backbone of the polynucleotide can comprise a polymer of synthetic subunits such as phosphoramidates and phorphorthioates and thus can be an oligodeoxynucleoside phosphoramidate (P—NH2) or a mixed phosphorothioate-phosphorodiester oligomer or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues can contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications can be deliberate, as through site-directed mutagenesis, or can be accidental, such as through mutations of hosts which produce the proteins or errors due to PCR amplification.

As used herein, the term “adjuvant” refers to a substance which modulates and/or engenders an immune response. Generally, the adjuvant is administered in conjunction with an antigen to effect enhancement of an immune response to the antigen as compared to antigen alone. Various adjuvants are described herein.

The terms “CpG oligodeoxynucleotide,” “CpG ODN,” and “ODN” herein refer to DNA molecules of 10 to 30 nucleotides in length containing a dinucleotide of cytosine and guanine separated by a phosphate (also referred to herein as a “CpG” dinucleotide or “CpG”). CpG ODNs of the present disclosure contain at least one unmethylated CpG dinucleotide. That is, the cytosine in the CpG dinucleotide is not methylated (i.e., is not 5-methylcytosine). CpG ODNs can have a partial or complete phosphorothioate (PS) backbone.

The term “pharmaceutically acceptable” or “pharmacologically compatible” as used herein is mean a material that is not biologically or otherwise undesirable, e.g., the material can be incorporated into a pharmaceutical composition administered to a patient without causing any significant undesirable biological effects or interacting in a deleterious manner with any of the other components of a composition in which it is contained. Pharmaceutically acceptable carriers or excipients have preferably met the required standards of toxicological and manufacturing testing and/or are included on the Inactive Ingredient Guide prepared by the U.S. Food and Drug Administration.

As used herein, the terms “peripheral blood mononuclear cells” or “PBMCs” refer to a heterogeneous population of blood cells having a round nucleus. Examples of cells that can be found in a population of PBMCs include lymphocytes such as T cells, B cells, NK cells (including natural killer T cells (NKT cells) and cytokine-induced killer cells (CIK cells)) and monocytes such as macrophages and dendritic cells.

The terms “enhanced antigen presenting cells” or “eAPCs” refer to immune cells that have been modified to exhibit enhanced function. For instance, as described herein, the eAPCs described herein are derived from PBMCs which have been modified to comprise one or more nucleic acid constructs (e.g., mRNA encoding HPV antigen, mRNA encoding a co-stimulatory molecule, and/or mRNA encoding a cytokine) using squeeze processing. The intracellular delivery of these constructs alters one or more properties of the PBMCs (e.g., expresses the encoded antigen on their surface such that they are capable of activating antigen-specific T cells and exhibits increased expression of a co-stimulatory molecule and/or cytokine), such that after the delivery, the modified cells are structurally and/or functionally different from the PBMCs, such that they are capable of enhancing the T cell response to the present antigens.

As used herein, “microfluidic systems” refers to systems in which low volumes (e.g., mL, nL, pL, fL) of fluids are processed to achieve the discrete treatment of small volumes of liquids. Certain implementations described herein include multiplexing, automation, and high throughput screening. The fluids (e.g., a buffer, solution, payload-containing solution, or cell suspension) can be moved, mixed, separated, or otherwise processed. In certain aspects, microfluidic systems are used to apply mechanical constriction to a cell suspended in a buffer, inducing perturbations in the cell (e.g., holes) that allow a payload or compound to enter the cytosol of the cell.

As used herein, a “constriction” can refer to a portion of a microfluidic channel defined by an entrance portion, a center point, and an exit portion, wherein the center point is defined by a width, a length, and a depth. In other examples, a constriction can refer to a pore or can be a portion of a pore. The pore can be contained on a surface (e.g., a filter and/or membrane).

For any of the structural and functional characteristics described herein, methods of determining these characteristics are known in the art.

### Methods of Treatment

In some aspects, provided are methods of treating a HPV-associated disease in a subject in need thereof, the method comprising administering to the subject a composition comprising enhanced APCs, wherein the enhanced APCs comprise an HPV antigen and are capable of activating T cells in an HLA agnostic manner. In some aspects, the method further comprises administering to the subject an immune checkpoint inhibitor.

In some aspects, provided are methods of treating a HPV-associated cancer in a subject in need thereof, the method comprising administering to the subject (i) a composition comprising enhanced APCs, wherein the enhanced APCs comprise an HPV antigen and are capable of activating T cells in an HLA agnostic manner, and (ii) an immune checkpoint inhibitor.

In some aspects, provided are methods of treating a HPV-associated disease in a subject in need thereof, the method comprising administering to the subject a composition comprising enhanced APCs, wherein the enhanced APCs are administered in an amount of about 0.25×10⁶ cells/kg to about 7.50×10⁶ cells/kg, and wherein the enhanced APCs comprise an HPV antigen and are capable of activating T cells in an HLA agnostic manner. In some aspects, the method further comprises administering to the subject an immune checkpoint inhibitor.

In some aspects, provided are methods for treating a HPV″ recurrent, locally advanced, or metastatic tumor in a subject in need thereof, the method comprising administering to the subject a composition comprising enhanced APCs, wherein the enhanced APCs comprise an HPV antigen and are capable of activating T cells in an HLA agnostic manner. In some aspects, the composition comprising enhanced APCs is administered in combination with an immune checkpoint inhibitor.

In some aspects, the HPV-associated cancer comprises a cervical cancer, anal cancer, vulval cancer, vaginal cancer, penile cancer, or oropharyngeal cancer. In some aspects, the HPV tumor comprises a cervical cancer, anal cancer, vulval cancer, vaginal cancer, penile cancer, or oropharyngeal cancer.

In some aspects, the subject is a human. In some aspects, the subject is an adult. In other aspects, the subject is an adolescent. In other aspects, the subject is a child. In some aspects, the subject is positive for human papillomavirus type 16 (HPV16*).

In some aspects, the enhanced APCs comprise T cells, B cells, NK cells, monocytes, or combinations thereof.

In some aspects, the enhanced APCs comprise an HPV antigen that comprises an HPV-16 antigen. In some aspects, the enhanced APCs comprise an HPV antigen that comprises an HPV-18 antigen. In some aspects, the HPV antigen comprises a peptide derived from HPV E6 (e.g., E6 protein of HPV-16 or HPV-18, which are both 158 amino acids in length). In some aspects, the HPV antigen comprises a peptide derived from HPV E7 (e.g., E7 protein of HPV-16, which is 98 amino acids in length, or E7 protein of HPV-18, which is 105 amino acids in length). In other aspects, the HPV antigen comprises a peptide derived from HPV E6 and a peptide derived from HPV E7. In some aspects, the peptide derived from HPV E6 is full-length E6 (e.g., E6 protein of HPV-16 or HPV-18, which are both 158 amino acids in length). In some aspects, the peptide derived from HPV E7 is full-length E7 (e.g., E7 protein of HPV-16, which is 98 amino acids in length, or E7 protein of HPV-18, which is 105 amino acids in length). In other aspects, the peptide derived from HPV E6 is full-length E6 and the peptide derived from HPV E7 is full-length HPV E7.

The amino acid sequence for the full-length E6 protein of HPV-16 is set forth in SEQ ID NO: 14 (see Table A below). The amino acid sequence for the full-length E7 protein of HPV-16 is set forth in SEQ ID NO: 15 (see Table A below). The amino acid sequence for the full-length E6 protein of HPV-18 is set forth in SEQ ID NO: 16 (see Table A below). The amino acid sequence for the full-length E7 protein of HPV-18 is set forth in SEQ ID NO: 17 (see Table A below).

TABLE A
Exemplary HPV E6 and E7 Protein Sequences

| | |
|---|---|
| HPV-16 E6 | MHQKRTAMFQDPQERPRKLPQLCTELQTTIHDIILECVYCKQQLLRREVY |
| UniProt: P03126 | DFAFRDLCIVYRDGNPYAVCDKCLKFYSKISEYRHYCYSLYGTTLEQQYN |
| (SEQ ID NO: 14) | KPLCDLLIRCINCOKPLCPEEKQRHLDKKQRFHNIRGRWTGRCMSCCRSS |
| | RTRRETQL |
| HPV-16 E7 | MHGDTPTLHEYMLDLQPETTDLYCYEQLNDSSEEEDEIDGPAGOAEPDRA |
| UniProt: P03129 | HYNIVTFCCKCDSTLRLCVOSTHVDIRTLEDLLMGTLGIVCPICSQKP |
| (SEQ ID NO: 15) | |
| HPV-18 E6 | MARFEDPTRRPYKLPDLCTELNTSLQDIEITCVYCKTVLELTEVFEFAFK |
| UniProt: P06463 | DLFVVYRDSIPHAACHKCIDFYSRIRELRHYSDSVYGDTLEKLINTGLYN |
| (SEQ ID NO: 16) | LLIRCLRCOKPLNPAEKLRHLNEKRRFHNIAGHYRGQCHSCCNRARQERL |
| | QRRRETQV |
| HPV-18 E7 | MHGPKATLQDIVLHLEPQNEIPVDLLCHEQLSDSEEENDEIDGVNHQHLP |
| UniProt: P06788 | ARRAEPQRHTMLCMCCKCEARIKLVVESSADDLRAFQQLFLNTLSFVCPW |
| (SEQ ID NO: 17) | CASQQ |

Accordingly, in some aspects, a method provided herein comprises administering a composition comprising enhanced APCs that comprise a HPV antigen, wherein the HPV antigen comprises the amino acid sequence set forth in SEQ ID NO: 14, and wherein the enhanced APCs are capable of activating T cells in an HLA-agnostic manner. In some aspects, a method provided herein comprises administering a composition comprising enhanced APCs that comprise a HPV antigen, wherein the HPV antigen comprises the amino acid sequence set forth in SEQ ID NO: 15, and wherein the enhanced APCs are capable of activating T cells in an HLA-agnostic manner. In some aspects, a method provided herein comprises administering a composition comprising enhanced APCs that comprise a HPV antigen, wherein the HPV antigen comprises the amino acid sequence set forth in SEQ ID NO: 16, and wherein the enhanced APCs are capable of activating T cells in an HLA-agnostic manner. In some aspects, a method provided herein comprises administering a composition comprising enhanced APCs that comprise a HPV antigen, wherein the HPV antigen comprises the amino acid sequence set forth in SEQ ID NO: 17, and wherein the enhanced APCs are capable of activating T cells in an HLA-agnostic manner.

In some aspects, the HPV antigen comprises a variant of the full-length HPV E6 protein (“HPV E6 variant”). In some aspects, the HPV E6 variant is a fragment of the full-length HPV E6 protein. For instance, in some aspects, the HPV E6 variant comprises about 5, about 10, about 20, about 30, about 40, about 50, about 60, about 70, about 80, about 90, about 100, about 110, about 120, about 130, about 140, about 150, or about 155-amino acid fragment of SEQ ID NO: 14 or SEQ ID NO: 16. In some aspects, the HPV E7 variant comprises about 5, about 10, about 20, about 30, about 40, about 50, about 60, about 70, about 80, about 90, or about 95-amino acid fragment of SEQ ID NO: 15. In some aspects, the HPV E7 variant comprises about 5, about 10, about 20, about 30, about 40, about 50, about 60, about 70, about 80, about 90, or about 100-amino acid fragment of SEQ ID NO: 17.

In some aspects, the HPV E6 variant comprises one or more amino acid substitutions as compared to the corresponding wild-type HPV E6 protein. Accordingly, in some aspects, the HPV E6 variant comprises one or more amino acid substitutions as compared to the sequence set forth in SEQ ID NO: 14. In some aspects, the HPV E6 variant comprises one or more amino acid substitutions as compared to the sequence set forth in SEQ ID NO: 16. In some aspects, the HPV E6 variant comprises an amino acid sequence that has at least about 75%, at least about 80%, at least about 85%, or about 99% sequence identity with the sequence set forth in SEQ ID NO: 14. In some aspects, the HPV E6 variant comprises an amino acid sequence that has at least about 75%, at least about 80%, at least about 85%, or about 99% sequence identity with the sequence set forth in SEQ ID NO: 16. Similarly, in some aspects, the HPV E7 variant comprises one or more amino acid substitutions as compared to the corresponding wild-type HPV E7 protein. In some aspects, the HPV E7 variant comprises one or more amino acid substitutions as compared to the sequence set forth in SEQ ID NO: 15. In some aspects, the HPV E7 variant comprises one or more amino acid substitutions as compared to the sequence set forth in SEQ ID NO: 17. In some aspects, the HPV E7 variant comprises an amino acid sequence that has at least about 75%, at least about 80%, at least about 85%, or about 99% sequence identity with the sequence set forth in SEQ ID NO: 15. In some aspects, the HPV E7 variant comprises an amino acid sequence that has at least about 75%, at least about 80%, at least about 85%, or about 99% sequence identity with the sequence set forth in SEQ ID NO: 17.

In some aspects, the enhanced APCs of a method provided herein comprises multiple antigens. In some aspects, the enhanced APCs comprise an HPV antigen and a non-HPV antigen. For instance, in some aspects, the enhanced APCs comprise multiple HPV antigens. In some aspects, the enhanced APCs comprise at least two, at least three, at least four, or at least five HPV antigens. In some aspects, the enhanced APCs comprises two HPV antigens, wherein the first HPV antigen is the HPV E6 protein (e.g., full-length E6 protein of HPV-16) and the second HPV antigen is the HPV E7 protein (e.g., full-length E7 protein of HPV-16).

In some aspects, any of the antigens described herein can be introduced to an input APC to produce the enhanced APCs using any suitable methods known in the art. Non-limiting examples of suitable methods for delivering one or more exogenous nucleotide sequences to a cell include: transfection (also known as transformation and transduction), electroporation, non-viral delivery, viral transduction, lipid nanoparticle delivery, and combinations thereof. In some aspects, an antigen (e.g., HPV antigen) is introduced to the input APCs using a constriction-mediated delivery described herein. As further described elsewhere in the present disclosure, as the cells pass through the constriction, they become transiently deformed, such that cell membrane of the cells is perturbed. The perturbation within the cell membrane can allow various payloads (e.g., nucleic acids encoding a HPV antigen, co-stimulatory molecule, and/or a cytokine) to enter the cells through the perturbation (e.g., through diffusion). The specific process by which the cells pass through a constriction and become transiently deformed is referred to herein as “squeeze processing,” “squeeze delivery,” or “squeezing.”

Accordingly, in some aspects, the enhanced cells described herein (e.g., comprising a HPV antigen) have been passed through a constriction under a set of parameters, thereby causing a perturbation within the APCs such that the antigen is able to enter the APCs through the perturbation when contacted with the APCs. More specifically, in some aspects, the enhanced APCs of a method described herein have been passed through a constriction under a set of parameters, thereby causing a perturbation within the APCs such that a nucleic acid encoding a HPV E6 protein (e.g., full-length E6 protein of HPV-16) enters the APCs when contacted with the APCs. In some aspects, the enhanced APCs of a method described herein have been passed through a constriction under a set of parameters, thereby causing a perturbation within the APCs such that a nucleic acid encoding a HPV E7 protein (e.g., full-length E7 protein of HPV-16) enters the APCs when contacted with the APCs. In some aspects, the enhanced APCs of a method described herein have been passed through a constriction under a set of parameters, thereby causing a perturbation within the APCs such that a nucleic acid encoding a HPV E6 protein (e.g., full-length E6 protein of HPV-16) and a nucleic acid encoding a HPV E7 protein (e.g., full-length E7 protein of HPV-16) enters the APCs when contacted with the APCs. In some aspects, the nucleic acid encoding the HPV E6 protein comprises an mRNA. In some aspects, the nucleic acid encoding the HPV E7 protein comprises an mRNA. In other aspects, the nucleic acid encoding the HPV E6 protein and the nucleic acid encoding the HPV E6 protein each comprise an mRNA.

In some aspects, enhanced APCs useful for the present disclosure further exhibit an increased expression of a co-stimulatory molecule as compared to corresponding APCs that have not been modified as described herein (“reference APCs”). In some aspects, enhanced APCs described herein further exhibit an increased expression of a cytokine as compared to the reference APCs. In some aspects, enhanced APCs described herein further exhibit an increased expression of both a co-stimulatory molecule and a cytokine as compared to the reference APCs.

As is generally understood in the art, for optimal T cell activation, multiple signals are required: (1) “signal 1”: antigen-specific signal provided by the binding of the TCR to antigenic peptide complexed with MHC; (2) “signal 2”: mediated by the engagement of co-stimulatory molecules such as CD80 and CD86 on antigen-presenting cells (APC); and (3) “signal 3”: mediated by cytokines (e.g., IL-2 and/or IL-12). Accordingly, compared to a method comprising administration of a composition that comprises reference APCs which have not been modified as described herein (e.g., does not exhibit increased expression of a co-stimulatory molecule and/or cytokine), a method comprising administration of a composition that comprises the enhanced APCs described herein is capable of inducing a much enhanced immune response. In some aspects, an enhanced immune response comprises: (i) an increase in the magnitude of the induced immune response as compared to that induced by the method comprising administration of a composition that comprises reference APCs, (ii) an increase in the breadth of the induced immune response as compared to that induced by the method comprising administration of a composition that comprises reference APCs, (iii) an increase in the duration of the induced immune response as compared to that induced by the method comprising administration of a composition that comprises reference APCs, or (iv) any combination of (i) to (iii).

In some aspects, the cytokine comprises a type I cytokine. In some aspects, the cytokine comprises IL-2, IL-4, IL-7, IL-10, IL-12, IL-15, IL-21, IL-1α, IL-1β, IL-1ra, IL-18, IL-33, IL-36α, IL-36β, IL-36γ, IL-36ra, IL-37, IL-38, IL-3, IL-5, IL-6, IL-11, IL-13, IL-23, granulocyte-macrophage colony stimulating factor (GM-CSF), granulocyte-colony stimulating factor (G-CSF), leukemia inhibitory factor (LIF), stem cell factor (SCF), thrombopoietin (TPO), macrophage-colony stimulating factor (M-CSF), erythropoieticn (EPO), Flt-3, IFN-α, IFN-β, IFN-γ, IL-19, IL-20, IL-22, IL-24, TNF-α, TNF-β, BAFF, APRIL, lymphotoxin beta (TNF-γ), IL-17A, IL-17B, IL-17C, IL-17D, IL-17E, IL-17F, IL-25, TSLP, IL-35, IL-27, TGF-β, or combinations thereof. In some aspects, the cytokine comprises IL-12. In some aspects, the cytokine comprises IL-2, In some aspects, the cytokine comprises both IL-12 and IL-2. In some aspects, the cytokine comprises a membrane bound form of a cytokine (“membrane-bound cytokine”). Amino acid sequences for exemplary membrane-bound cytokines are set forth in SEQ ID NOs: 7-10 and 13.

In some aspects, the co-stimulatory molecule comprises OX40, OX40L, CD27, CD70, CD40, CD40L, 4-1BB, 4-1BBL, CD28, CD80, CD86, ICOS, ICOSL, or related molecules thereof, or any combination thereof. In some aspects, the co-stimulatory molecule comprises CD80. In some aspects, the co-stimulatory molecule comprises CD86.

Accordingly, as is apparent from at least the above disclosure, provided herein is a method that comprises administering a composition comprising enhanced APCs comprising an HPV antigen, wherein the enhanced APCs exhibit increased expression of a co-stimulatory molecule and/or a cytokine as compared to corresponding APCs that have not been modified (e.g., as described herein), and wherein the enhanced APCs are capable of activating T cells in a HLA-agnostic manner. More specifically, in some aspects, a method as described herein comprises administering a composition comprising enhanced APCs comprising an HPV E6 protein or variant thereof (e.g., full-length HPV-16 E6 protein) and/or an HPV E7 protein or variant thereof (e.g., full-length HPV-16 E7 protein), wherein the enhanced APCs exhibit increased expression of a co-stimulatory molecule (e.g., CD86) and a cytokine (e.g., membrane-bound IL-2 and/or membrane-bound IL-12) as compared to corresponding reference APCs that have not been modified (e.g., as described herein), and wherein the enhanced APCs are capable of activating T cells in a HLA-agnostic manner.

Not to be bound by any one theory, in some aspects, a nucleic acid encoding a co-stimulatory molecule and/or a nucleic acid encoding a cytokine can be introduced into APCs to modify them to exhibit increased expression of the co-stimulatory molecule and/or cytokine. In some aspects, such nucleic acids can be introduced into the APCs using a constriction-mediated delivery described herein. In some aspects, the enhanced cells described herein (e.g., comprising a HPV antigen) have been passed through a constriction under a set of parameters, thereby causing a perturbation within the APCs such that any of the following nucleic acids enter the APCs through the perturbation when contacted with the APCs: (i) a nucleic acid encoding a HPV E7 protein (e.g., full-length E7 protein of HPV-16), (ii) a nucleic acid encoding a HPV E6 protein (e.g., full-length E6 protein of HPV-16), (iii) a nucleic acid encoding a co-stimulatory molecule (e.g., CD86), (iv) a nucleic acid encoding a cytokine (e.g., membrane-bound IL-2 and/or membrane-bound IL-12), or (v) any combination of (i) to (iv). In some aspects, the nucleic acid comprises an mRNA.

In some aspects, any of the enhanced APCs provided herein (e.g., comprising an HPV antigen and/or exhibiting increased expression of a co-stimulatory molecule and/or cytokine) can be conditioned, such that APCs exhibit improved properties compared to corresponding APCs that have not been conditioned. As is apparent from the present disclosure, in some aspects, the enhanced APCs described herein have been incubated in the presence of an adjuvant, such that the APCs are conditioned. In some aspects, the enhanced APCs described herein have been incubated with an adjuvant for about 2 hours to about 10 hours, about 2 hours to about 9 hours, about 2 hours to about 8 hours, about 2 hours to about 7 hours, about 2 hours to about 6 hours, about 2 hours to about 5 hours, about 2 hours to about 4 hours, about 3 hours to about 10 hours, about 3 hours to about 9 hours, about 3 hours to about 8 hours, about 3 hours to about 7 hours, about 3 hours to about 6 hours, about 3 hours to about 5 hours, about 3 hours to about 4 hours, about 4 hours to about 10 hours, about 4 hours to about 9 hours, about 4 hours to about 8 hours, about 4 hours to about 7 hours, about 4 hours to about 6 hours, about 4 hours to about 5 hours, about 5 hours to about 10 hours, about 5 hours to about 9 hours, about 5 hours to about 8 hours, about 5 hours to about 7 hours, about 5 hours to about 6 hours, about 6 hours to about 10 hours, about 6 hours to about 9 hours, about 6 hours to about 8 hours, about 6 hours to about 7 hours, about 7 hours to about 10 hours, about 7 hours to about 9 hours, about 7 hours to about 8 hours, about 8 hours to about 10 hours, or about 8 hours to about 9 hours. In some aspects, the enhanced APCs described herein have been incubated with an adjuvant for about 3 hours to about 6 hours. In some aspects, the enhanced APCs described herein have been incubated with an adjuvant for about 2 hours, about 3 hours, about 4 hours, about 5 hours, about 6 hours, about 7 hours, about 8 hours, about 9 hours, or about 10 hours. In some aspects, the enhanced APCs described herein have been incubated with an adjuvant for about 4 hours. In some aspects, the incubation is carried out at about 37° C.

Non-limiting examples of adjuvants useful for the present disclosure comprises: stimulator of interferon genes (STING) agonists, retinoic acid-inducible gene I (RIG-I) agonists, and agonists for TLR3, TLR4, TLR7, TLR8, TLR9, CpG ODN, interferon-α (IFN-α), IFN-β, IFN-γ, alpha-galactosyl ceramide, polyinosinic: polycytidylic acid (polyI:C), imiquimod (R837), resiquimod (R848), cyclic dinucleotides (CDN), or lipopolysaccharide (LPS). In some aspects, the CpG ODN comprises a Class A CpG ODN, a Class B CpG ODN, or a Class C CpG ODN. In some aspects, the CpG ODN comprises CpG ODN 1018, CpG ODN 1585, CpG ODN 2216, CpG ODN 2336, CpG ODN 1668, CpG ODN 1826, CPG ODN 2006, CpG ODN 2007, CpG ODN BW006, CpG ODN D-SL01, CpG ODN 2395, CpG ODN M362, CpG ODN D-SL03.

In some aspects, the enhanced APCs of a method described herein have been prepared by passing a cell suspension comprising input APCs through a cell-deforming constriction, thereby causing a perturbation within the APCs such that a nucleic acid encoding a HPV E6 protein (e.g., full-length E6 protein of HPV-16) enters the APCs when contacted with the APCs. In some aspects, the enhanced APCs of a method described herein have been prepared by passing a cell suspension comprising input APCs through a cell-deforming constriction, thereby causing a perturbation within the APCs such that a nucleic acid encoding a HPV E7 protein (e.g., full-length E7 protein of HPV-16) enters the APCs when contacted with the APCs. In some aspects, the enhanced APCs of a method described herein have been prepared by passing a cell suspension comprising input APCs through a cell-deforming constriction, thereby causing a perturbation within the APCs such that a nucleic acid encoding a HPV E6 protein (e.g., full-length E6 protein of HPV-16) and a nucleic acid encoding a HPV E7 protein (e.g., full-length E7 protein of HPV-16) enters the APCs when contacted with the APCs. In some aspects, the cell suspension comprising the input APCs have been cultured in combination with the nucleic acid encoding the antigen, the nucleic acid encoding the co-stimulatory molecule, and/or the nucleic acid encoding the cytokine, such that the nucleic acid encoding the antigen, the nucleic acid encoding the co-stimulatory molecule, and/or the nucleic acid encoding the cytokine are in contact with the input APCs, thereby producing enhanced APCs capable of inducing an enhanced immune response. In some aspects, the nucleic acid encoding the antigen, the nucleic acid encoding the co-stimulatory molecule, and/or the nucleic acid encoding the cytokine is an mRNA.

In some aspects, the cell suspension comprising the input APCs are passed through multiple cell-deforming constrictions, wherein the multiple cell-deforming constrictions are arranged in series and/or in parallel. In some aspects, the cell-deforming constriction comprises a diameter which is about 4.2 μm to about 6 μm, about 4.2 μm to about 5.8 μm, about 4.2 μm to about 5.6 μm, about 4.2 μm to about 5.4 μm, about 4.2 μm to about 5.2 μm, about 4.2 μm to about 5.0 μm, or about 4.2 μm to about 4.8 μm. In some aspects, the cell-deforming constriction comprises a diameter which is about or less than any one of 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, 10.5 μm, 11 μm, 11.5 μm, 12 μm, 12.5 μm, 13 μm, 13.5 μm, 14 μm, 14.5 μm, or 15 μm. In some aspects, the cell-deforming constriction comprises a diameter which is about or less than any one of 3.0 μm, 3.1 μm, 3.2 μm, 3.3 μm, 3.4 μm, 3.5 μm, 3.6 μm, 3.7 μm, 3.8 μm, 3.9 μm, 4.0 μm, 4.1 μm, 4.2 μm, 4.3 μm, 4.4 μm, 4.5 μm, 4.6 μm, 4.7 μm, 4.8 μm, 4.9 μm, or 5.0 μm. In some aspects, the cell-deforming constriction comprises a diameter which is about 4.5 μm.

### Doses and Regimens

In some aspects, provided are methods for treating a HPV-associated cancer in a subject in need thereof, the method comprising administering to the subject a composition comprising enhanced APCs, wherein the enhanced APCs are administered in an amount of about 0.25×10⁶ cells/kg to about 7.50×10⁶ cells/kg, and wherein the enhanced APCs comprise an HPV antigen and are capable of activating T cells in an HLA agnostic manner. In some aspects, the method further comprises administering an immune checkpoint inhibitor.

In some aspects, the enhanced APCs are administered in an amount of any one of about 0.25×10⁶ cells/kg to about 7.50×10⁶ cells/kg, about 0.25×10⁶ cells/kg to about 5.0×10⁶ cells/kg, about 0.25×10⁶ cells/kg to about 3.25×10⁶ cells/kg, about 0.25×10⁶ cells/kg to about 2.5×10⁶ cells/kg, about 0.25×10⁶ cells/kg to about 1.25×10⁶ cells/kg, about 0.25×10⁶ cells/kg to about 0.5×10⁶ cells/kg, about 0.5×10⁶ cells/kg to about 7.50×10⁶ cells/kg, about 0.5×10⁶ cells/kg to about 5.0×10⁶ cells/kg, about 0.5×10⁶ cells/kg to about 3.25×10⁶ cells/kg, about 0.5×10⁶ cells/kg to about 2.5×10⁶ cells/kg, about 0.5×10⁶ cells/kg to about 1.25×10⁶ cells/kg, about 1.25×10⁶ cells/kg to about 7.50×10⁶ cells/kg, about 1.25×10⁶ cells/kg to about 5.0×10⁶ cells/kg, about 1.25×10⁶ cells/kg to about 3.25×10⁶ cells/kg, about 1.25×10⁶ cells/kg to about 2.5×10⁶ cells/kg, about 2.5×10⁶ cells/kg to about 7.50×10⁶ cells/kg, about 2.5×10⁶ cells/kg to about 5.0×10⁶ cells/kg, about 2.5×10⁶ cells/kg to about 3.25×10⁶ cells/kg, about 3.25×10⁶ cells/kg to about 7.50×10⁶ cells/kg, about 3.25×10⁶ cells/kg to about 5.0×10⁶ cells/kg, and about 5.0×10⁶ cells/kg to about 7.50×10⁶ cells/kg. In some aspects, the enhanced APCs are administered in an amount of about 0.25×10⁶ cells/kg to about 7.50×10⁶ cells/kg. In other aspects, the enhanced APCs are administered in an amount of about 0.5×10⁶ cells/kg to about 5.0×10⁶ cells/kg. In some aspects, the enhanced APCs are administered in an amount of any one of about 0.25×10⁶ cells/kg, about 0.5×10⁶ cells/kg, about 1.25×10⁶ cells/kg, about 2.5×10⁶ cells/kg, about 3.25×10⁶ cells/kg, about 5.0×10⁶ cells/kg, and about 7.50×10⁶ cells/kg.

In some aspects, where an immune checkpoint inhibitor is administered to the subjects, that the immune checkpoint inhibitor comprises an antagonist of PD-1 (such as but not limited to pembrolizumab), an antagonist of PD-L1 (such as but not limited to atezolizumab), and/or an antagonist of CTLA-4 (such as but not limited to ipilimumab). Accordingly, in some aspects, disclosed herein is a method of treating a human papilloma virus (HPV)-associated cancer in a subject in need thereof, comprising administering to the subject a composition comprising the enhanced APCs described herein and an antagonist of PD-1. In some aspects, disclosed herein is a method of treating a human papilloma virus (HPV)-associated cancer in a subject in need thereof, comprising administering to the subject a composition comprising the enhanced APCs described herein and an antagonist of PD-L1. In some aspects, disclosed herein is a method of treating a human papilloma virus (HPV)-associated cancer in a subject in need thereof, comprising administering to the subject a composition comprising the enhanced APCs described herein and an antagonist of CTLA-4. In some aspects, the antagonist of PD-1 is an antibody that binds PD-1. In some aspects, the antagonist of PD-L1 is an antibody that binds PD-L1. In some aspects, the antagonist of CTLA-4 is an antibody that binds CTLA-4.

In some aspects, the antibody that binds PD-1 is pembrolizumab. In some aspects, pembrolizumab is administered in an amount of any one of about 10 mg to about 400 mg, about 10 mg to about 390 mg about 10 mg to about 380 mg, about 10 mg to about 370 mg, about 10 mg to about 360 mg, about 10 mg to about 350 mg, about 10 mg to about 340 mg, about 10 mg to about 330 mg, about 10 mg to about 320 mg, about 10 mg to about 310 mg, about 10 mg to about 300 mg, about 10 mg to about 290 mg, about 10 mg to about 280 mg, about 10 mg to about 270 mg, about 10 mg to about 260 mg, about 10 mg to about 250 mg, about 10 mg to about 240 mg, about 10 mg to about 230 mg, about 10 mg to about 220 mg, about 10 mg to about 210 mg, about 10 mg to about 200 mg, about 10 mg to about 150 mg, about 10 mg to about 100 mg, about 10 mg to about 50 mg, about 50 mg to about 400 mg, about 50 mg to about 390 mg about 50 mg to about 380 mg, about 50 mg to about 370 mg, about 50 mg to about 360 mg, about 50 mg to about 350 mg, about 50 mg to about 340 mg, about 50 mg to about 330 mg, about 50 mg to about 320 mg, about 50 mg to about 310 mg, about 50 mg to about 300 mg, about 50 mg to about 290 mg, about 50 mg to about 280 mg, about 50 mg to about 270 mg, about 50 mg to about 260 mg, about 50 mg to about 250 mg, about 50 mg to about 240 mg, about 50 mg to about 230 mg, about 50 mg to about 220 mg, about 50 mg to about 210 mg, about 50 mg to about 200 mg, about 50 mg to about 150 mg, about 50 mg to about 100 mg, about 100 mg to about 400 mg, about 100 mg to about 390 mg about 100 mg to about 380 mg, about 100 mg to about 370 mg, about 100 mg to about 360 mg, about 100 mg to about 350 mg, about 100 mg to about 340 mg, about 100 mg to about 330 mg, about 100 mg to about 320 mg, about 100 mg to about 310 mg, about 100 mg to about 300 mg, about 100 mg to about 290 mg, about 100 mg to about 280 mg, about 100 mg to about 270 mg, about 100 mg to about 260 mg, about 100 mg to about 250 mg, about 100 mg to about 240 mg, about 100 mg to about 230 mg, about 100 mg to about 220 mg, about 100 mg to about 210 mg, about 100 mg to about 200 mg, about 100 mg to about 150 mg, about 150 mg to about 400 mg, about 150 mg to about 390 mg about 150 mg to about 380 mg, about 150 mg to about 370 mg, about 150 mg to about 360 mg, about 150 mg to about 350 mg, about 150 mg to about 340 mg, about 150 mg to about 330 mg, about 150 mg to about 320 mg, about 150 mg to about 310 mg, about 150 mg to about 300 mg, about 150 mg to about 290 mg, about 150 mg to about 280 mg, about 150 mg to about 270 mg, about 150 mg to about 260 mg, about 150 mg to about 250 mg, about 150 mg to about 240 mg, about 150 mg to about 230 mg, about 150 mg to about 220 mg, about 150 mg to about 210 mg, about 150 mg to about 200 mg, about 200 mg to about 400 mg, about 200 mg to about 390 mg about 200 mg to about 380 mg, about 200 mg to about 370 mg, about 200 mg to about 360 mg, about 200 mg to about 350 mg, about 200 mg to about 340 mg, about 200 mg to about 330 mg, about 200 mg to about 320 mg, about 200 mg to about 310 mg, about 200 mg to about 300 mg, about 200 mg to about 290 mg, about 200 mg to about 280 mg, about 200 mg to about 270 mg, about 200 mg to about 260 mg, about 200 mg to about 250 mg, about 200 mg to about 240 mg, about 200 mg to about 230 mg, about 200 mg to about 220 mg, about 200 mg to about 210 mg, about 225 mg to about 400 mg, about 225 mg to about 390 mg about 225 mg to about 380 mg, about 225 mg to about 370 mg, about 225 mg to about 360 mg, about 225 mg to about 350 mg, about 225 mg to about 340 mg, about 225 mg to about 330 mg, about 225 mg to about 320 mg, about 225 mg to about 310 mg, about 225 mg to about 300 mg, about 225 mg to about 290 mg, about 225 mg to about 280 mg, about 225 mg to about 270 mg, about 225 mg to about 260 mg, about 225 mg to about 250 mg, about 250 mg to about 400 mg, about 250 mg to about 390 mg about 250 mg to about 380 mg, about 250 mg to about 370 mg, about 250 mg to about 360 mg, about 250 mg to about 350 mg, about 250 mg to about 340 mg, about 250 mg to about 330 mg, about 250 mg to about 320 mg, about 250 mg to about 310 mg, about 250 mg to about 300 mg, about 250 mg to about 275 mg, about 275 mg to about 400 mg, about 275 mg to about 390 mg about 275 mg to about 380 mg, about 275 mg to about 370 mg, about 275 mg to about 360 mg, about 275 mg to about 350 mg, about 275 mg to about 340 mg, about 275 mg to about 330 mg, about 275 mg to about 320 mg, about 275 mg to about 310 mg, about 275 mg to about 300 mg, about 300 mg to about 400 mg, about 300 mg to about 390 mg about 300 mg to about 380 mg, about 300 mg to about 370 mg, about 300 mg to about 360 mg, about 300 mg to about 350 mg, about 300 mg to about 325 mg, about 325 mg to about 400 mg, about 325 mg to about 390 mg about 325 mg to about 380 mg, about 325 mg to about 370 mg, about 325 mg to about 360 mg, about 325 mg to about 350 mg, about 350 mg to about 400 mg, about 350 mg to about 390 mg about 350 mg to about 380 mg, about 350 mg to about 375 mg, and about 375 mg to about 400 mg. In some aspects, pembrolizumab is administered in an amount of any one of about 10 mg, about 50 mg, about 100 mg, about 150 mg, about 200 mg, about 210 mg, about 220 mg, about 225 mg, about 230 mg, about 240 mg, about 250 mg, about 260 mg, about 270 mg, about 275 mg, about 280 mg, about 290 mg, about 300 mg, about 310 mg, about 320 mg, about 325 mg, about 330 mg, about 340 mg, about 350 mg, about 360 mg, about 370 mg, about 375 mg, about 380 mg, about 390 mg, and about 400 mg. In some aspects, the pembrolizumab is administered in an amount of about 200 mg.

In some aspects, the antibody that binds PD-1 is nivolumab. In some aspects, nivolumab is administered in an amount of any one of about 15 mg to about 500 mg, about 15 mg to about 480 mg, about 15 mg to about 440 mg, about 15 mg to about 400 mg, about 15 mg to about 360 mg, about 15 mg to about 320 mg, about 15 mg to about 300 mg, about 15 mg to about 280 mg, about 15 mg to about 240 mg, about 15 mg to about 200 mg, about 15 mg to about 160 mg, about 15 mg to about 120 mg, about 15 mg to about 100 mg, about 15 mg to about 40 mg, about 40 mg to about 500 mg, about 40 mg to about 480 mg, about 40 mg to about 440 mg, about 40 mg to about 400 mg, about 40 mg to about 360 mg, about 40 mg to about 320 mg, about 40 mg to about 300 mg, about 40 mg to about 280 mg, about 40 mg to about 240 mg, about 40 mg to about 200 mg, about 40 mg to about 160 mg, about 40 mg to about 120 mg, about 40 mg to about 100 mg, about 100 mg to about 500 mg, about 100 mg to about 480 mg, about 100 mg to about 440 mg, about 100 mg to about 400 mg, about 100 mg to about 360 mg, about 100 mg to about 320 mg, about 100 mg to about 300 mg, about 100 mg to about 280 mg, about 100 mg to about 240 mg, about 100 mg to about 200 mg, about 100 mg to about 160 mg, about 100 mg to about 120 mg, about 120 mg to about 500 mg, about 120 mg to about 480 mg, about 120 mg to about 440 mg, about 120 mg to about 400 mg, about 120 mg to about 360 mg, about 120 mg to about 320 mg, about 120 mg to about 300 mg, about 120 mg to about 280 mg, about 120 mg to about 240 mg, about 120 mg to about 200 mg, about 120 mg to about 160 mg, about 160 mg to about 500 mg, about 160 mg to about 480 mg, about 160 mg to about 440 mg, about 160 mg to about 400 mg, about 160 mg to about 360 mg, about 160 mg to about 320 mg, about 160 mg to about 300 mg, about 160 mg to about 280 mg, about 160 mg to about 240 mg, about 160 mg to about 200 mg, about 200 mg to about 500 mg, about 200 mg to about 480 mg, about 200 mg to about 440 mg, about 200 mg to about 400 mg, about 200 mg to about 360 mg, about 200 mg to about 320 mg, about 200 mg to about 300 mg, about 200 mg to about 280 mg, about 200 mg to about 240 mg, about 240 mg to about 500 mg, about 240 mg to about 480 mg, about 240 mg to about 440 mg, about 240 mg to about 400 mg, about 240 mg to about 360 mg, about 240 mg to about 320 mg, about 240 mg to about 300 mg, about 240 mg to about 280 mg, about 280 mg to about 500 mg, about 280 mg to about 480 mg, about 280 mg to about 440 mg, about 280 mg to about 400 mg, about 280 mg to about 360 mg, about 280 mg to about 320 mg, about 280 mg to about 300 mg, about 300 mg to about 500 mg, about 300 mg to about 480 mg, about 300 mg to about 440 mg, about 300 mg to about 400 mg, about 300 mg to about 360 mg, about 300 mg to about 320 mg, about 320 mg to about 500 mg, about 320 mg to about 480 mg, about 320 mg to about 440 mg, about 320 mg to about 400 mg, about 320 mg to about 360 mg, about 360 mg to about 500 mg, about 360 mg to about 480 mg, about 360 mg to about 440 mg, about 360 mg to about 400 mg, about 360 mg to about 500 mg, about 360 mg to about 480 mg, about 360 mg to about 440 mg, about 360 mg to about 400 mg, about 400 mg to about 500 mg, about 400 mg to about 480 mg, about 400 mg to about 440 mg, about 440 mg to about 500 mg, about 440 mg to about 480 mg, or about 480 mg to about 500 mg. In some aspects, nivolumab is administered in an amount of any one of about 15 mg, about 40 mg, about 100 mg, about 120 mg, about 160 mg, about 200 mg, about 240 mg, about 280 mg, about 300 mg, about 320 mg, about 360 mg, about 400 mg, about 440 mg, about 480 mg, and about 500 mg. In some aspects, the nivolumab is administered in an amount of about 360 mg.

In some aspects, the antibody that binds PD-L1 is atezolizumab. In some aspects, atezolizumab is administered in an amount of any one of about 75 mg to about 1700 mg, about 75 mg to about 1680 mg, about 75 mg to about 1600 mg, about 75 mg to about 1500 mg, about 75 mg to about 1400 mg, about 75 mg to about 1300 mg, about 75 mg to about 1200 mg, about 75 mg to about 1080 mg, about 75 mg to about 900 mg, about 75 mg to about 840 mg, about 75 mg to about 720 mg, about 75 mg to about 600 mg, about 75 mg to about 480 mg, about 75 mg to about 360 mg, about 75 mg to about 300 mg, about 75 mg to about 240 mg, about 75 mg to about 120 mg, about 120 mg to about 1700 mg, about 120 mg to about 1680 mg, about 120 mg to about 1600 mg, about 120 mg to about 1500 mg, about 120 mg to about 1400 mg, about 120 mg to about 1300 mg, about 120 mg to about 1200 mg, about 120 mg to about 1080 mg, about 120 mg to about 900 mg, about 120 mg to about 840 mg, about 120 mg to about 720 mg, about 120 mg to about 600 mg, about 120 mg to about 480 mg, about 120 mg to about 360 mg, about 120 mg to about 300 mg, about 120 mg to about 240 mg, about 240 mg to about 1700 mg, about 240 mg to about 1680 mg, about 240 mg to about 1600 mg, about 240 mg to about 1500 mg, about 240 mg to about 1400 mg, about 240 mg to about 1300 mg, about 240 mg to about 1200 mg, about 240 mg to about 1080 mg, about 240 mg to about 900 mg, about 240 mg to about 840 mg, about 240 mg to about 720 mg, about 240 mg to about 600 mg, about 240 mg to about 480 mg, about 240 mg to about 360 mg, about 240 mg to about 300 mg, about 300 mg to about 1700 mg, about 300 mg to about 1680 mg, about 300 mg to about 1600 mg, about 300 mg to about 1500 mg, about 300 mg to about 1400 mg, about 300 mg to about 1300 mg, about 300 mg to about 1200 mg, about 300 mg to about 1080 mg, about 300 mg to about 900 mg, about 300 mg to about 840 mg, about 300 mg to about 720 mg, about 300 mg to about 600 mg, about 300 mg to about 480 mg, about 300 mg to about 360 mg, about 360 mg to about 1700 mg, about 360 mg to about 1680 mg, about 360 mg to about 1600 mg, about 360 mg to about 1500 mg, about 360 mg to about 1400 mg, about 360 mg to about 1300 mg, about 360 mg to about 1200 mg, about 360 mg to about 1080 mg, about 360 mg to about 900 mg, about 360 mg to about 840 mg, about 360 mg to about 720 mg, about 360 mg to about 600 mg, about 360 mg to about 480 mg, about 480 mg to about 1700 mg, about 480 mg to about 1680 mg, about 480 mg to about 1600 mg, about 480 mg to about 1500 mg, about 480 mg to about 1400 mg, about 480 mg to about 1300 mg, about 480 mg to about 1200 mg, about 480 mg to about 1080 mg, about 480 mg to about 900 mg, about 480 mg to about 840 mg, about 480 mg to about 720 mg, about 480 mg to about 600 mg, about 600 mg to about 1700 mg, about 600 mg to about 1680 mg, about 600 mg to about 1600 mg, about 600 mg to about 1500 mg, about 600 mg to about 1400 mg, about 600 mg to about 1300 mg, about 600 mg to about 1200 mg, about 600 mg to about 1080 mg, about 600 mg to about 900 mg, about 600 mg to about 840 mg, about 600 mg to about 720 mg, about 720 mg to about 1700 mg, about 720 mg to about 1680 mg, about 720 mg to about 1600 mg, about 720 mg to about 1500 mg, about 720 mg to about 1400 mg, about 720 mg to about 1300 mg, about 720 mg to about 1200 mg, about 720 mg to about 1080 mg, about 720 mg to about 900 mg, about 720 mg to about 840 mg, about 840 mg to about 1700 mg, about 840 mg to about 1680 mg, about 840 mg to about 1600 mg, about 840 mg to about 1500 mg, about 840 mg to about 1400 mg, about 840 mg to about 1300 mg, about 840 mg to about 1200 mg, about 840 mg to about 1080 mg, about 840 mg to about 900 mg, about 900 mg to about 1700 mg, about 900 mg to about 1680 mg, about 900 mg to about 1600 mg, about 900 mg to about 1500 mg, about 900 mg to about 1400 mg, about 900 mg to about 1300 mg, about 900 mg to about 1200 mg, about 900 mg to about 1080 mg, about 1080 mg to about 1700 mg, about 1080 mg to about 1680 mg, about 1080 mg to about 1600 mg, about 1080 mg to about 1500 mg, about 1080 mg to about 1400 mg, about 1080 mg to about 1300 mg, about 1080 mg to about 1200 mg, about 1200 mg to about 1700 mg, about 1200 mg to about 1680 mg, about 1200 mg to about 1600 mg, about 1200 mg to about 1500 mg, about 1200 mg to about 1400 mg, about 1200 mg to about 1300 mg, about 1300 mg to about 1700 mg, about 1300 mg to about 1680 mg, about 1300 mg to about 1600 mg, about 1300 mg to about 1500 mg, about 1300 mg to about 1400 mg, about 1400 mg to about 1700 mg, about 1400 mg to about 1680 mg, about 1400 mg to about 1600 mg, about 1400 mg to about 1500 mg, about 1500 mg to about 1700 mg, about 1500 mg to about 1680 mg, about 1500 mg to about 1600 mg, about 1600 mg to about 1700 mg, about 1600 mg to about 1680 mg, or about 1680 mg to about 1700 mg. In some aspects, atezolizumab is administered in an amount of any one of about 75 mg, about 120 mg, about 240 mg, about 300 mg, about 360 mg, about 480 mg, about 600 mg, about 720 mg, about 840 mg, about 900 mg, about 1080 mg, about 1200 mg, about 1300 mg, about 1400 mg, about 1500 mg, about 1600 mg, about 1680 mg, and about 1700 mg. In some aspects, atezolizumab is administered in an amount of about 1200 mg.

In some aspects, the antibody that binds CTLA-4 is ipilimumab. In some aspects, ipilimumab is administered in an amount of any one of about 1 mg/kg to about 10 mg/kg, about 1 mg/kg to about 9 mg/kg, about 1 mg/kg to about 8 mg/kg, about 1 mg/kg to about 7 mg/kg, about 1 mg/kg to about 6 mg/kg, about 1 mg/kg to about 5 mg/kg, about 1 mg/kg to about 4 mg/kg, about 1 mg/kg to about 3 mg/kg, about 1 mg/kg to about 2 mg/kg, about 2 mg/kg to about 10 mg/kg, about 2 mg/kg to about 9 mg/kg, about 2 mg/kg to about 8 mg/kg, about 2 mg/kg to about 7 mg/kg, about 2 mg/kg to about 6 mg/kg, about 2 mg/kg to about 5 mg/kg, about 2 mg/kg to about 4 mg/kg, about 2 mg/kg to about 3 mg/kg, about 3 mg/kg to about 10 mg/kg, about 3 mg/kg to about 9 mg/kg, about 3 mg/kg to about 8 mg/kg, about 3 mg/kg to about 7 mg/kg, about 3 mg/kg to about 6 mg/kg, about 3 mg/kg to about 5 mg/kg, about 3 mg/kg to about 4 mg/kg, about 4 mg/kg to about 10 mg/kg, about 4 mg/kg to about 9 mg/kg, about 4 mg/kg to about 8 mg/kg, about 4 mg/kg to about 7 mg/kg, about 4 mg/kg to about 6 mg/kg, about 4 mg/kg to about 5 mg/kg, about 5 mg/kg to about 10 mg/kg, about 5 mg/kg to about 9 mg/kg, about 5 mg/kg to about 8 mg/kg, about 5 mg/kg to about 7 mg/kg, about 5 mg/kg to about 6 mg/kg, about 6 mg/kg to about 10 mg/kg, about 6 mg/kg to about 9 mg/kg, about 6 mg/kg to about 8 mg/kg, about 6 mg/kg to about 7 mg/kg, about 7 mg/kg to about 10 mg/kg, about 7 mg/kg to about 9 mg/kg, about 7 mg/kg to about 8 mg/kg, about 8 mg/kg to about 10 mg/kg, and about 8 mg/kg to about 9 mg/kg. In some aspects, ipilimumab is administered in an amount of about 1 mg/kg to about 3 mg/kg. In some aspects, ipilimumab is administered in an amount of any one of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, and about 10 mg/kg. In some aspects, ipilimumab is administered in an amount of about 3 mg/kg.

In some aspects, according to any one of the methods described herein, the composition comprising enhanced APCs is administered intravenously. In some aspects, the immune checkpoint inhibitor is administered intravenously, orally, or subcutaneously.

In some aspects, a method described herein comprises multiple (e.g., any of 2, 3, 4, 5, 6, 7, 8, 9, 10 or more) cycles of administering the composition comprising enhanced APCs as described herein to the subject in need thereof. In some aspects, the enhanced APCs are administered to the subject in need thereof 2, 3, 4, 5, 6, 7, 8, 9, 10 or more times. In some aspects, the duration of time between any two consecutive administrations of the composition comprising enhanced APCs is at least about 1 day (e.g., at least about any of 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 1 year, or longer, including any ranges between these values).

In some aspects, the composition comprising enhanced APCs is administered in any one of a 1-, 2-, 3—, 4-, 5-, 6-, 7-, 8-, 9-, or 10-week cycle. In some aspects, the composition comprising enhanced APCs is administered on day 1 of any one of a 1-, 2-, 3—, 4-, 5-, 6-, 7-, 8-, 9-, or 10-week cycle. In some aspects, the composition comprising enhanced APCs is administered in a 3-week cycle. In some aspects, the composition comprising enhanced APCs is administered in a 6-week cycle. In some aspects, the composition comprising enhanced APCs is administered on one or more of day 1, 2, 3, 4, 5, 6, 7, 8, 9, 10, 11, 12, 13, 14, 15, 16, 17, 18, 19, or 20 in a treatment cycle. In some aspects, the composition comprising enhanced APCs is administered on day 1 of a treatment cycle. In other aspects, the composition comprising enhanced APCs is administered on day 2 of a treatment cycle. In some aspects, the composition comprising enhanced APCs is administered on day 1 and day 2 of a treatment cycle. In some aspects, the composition comprising enhanced APCs is administered on day 1 and day 3 of a treatment cycle. In some aspects, the composition comprising enhanced APCs is administered on day 8 of a treatment cycle. In some aspects, the composition comprising enhanced APCs is administered on day 1 of a three-week cycle. In some aspects, the composition comprising enhanced APCs is further administered on day 2 of a three-week cycle. In some aspects, the composition comprising enhanced APCs is administered in three-week cycles until the supply of the composition of enhanced APCs is exhausted, or for one year. In some aspects, the composition comprising enhanced APCs is administered to the subject for at least about 3 months, 6 months, 9 months, 12 months, 15 months, 18 months, 21 months, or 24 months.

In some aspects, any one of about 0.25×10⁶ cells/kg, about 0.5×10⁶ cells/kg, about 1.25×10⁶ cells/kg, about 2.5×10⁶ cells/kg, about 3.25×10⁶ cells/kg, about 5.0×10⁶ cells/kg, and about 7.50×10⁶ cells/kg of enhanced APCs are administered on day 1 of each three-week cycle. In some aspects, about 0.25×10⁶ cells/kg to about 7.5×10⁶ cells/kg of enhanced APCs are administered on day 1 of each three-week cycle. In some aspects, about 0.5×10⁶ cells/kg to about 5.0×10⁶ cells/kg of enhanced APCs are administered on day 1 of each three-week cycle. In some aspects, about 0.5×10⁶ cells/kg, about 2.5×10⁶ cells/kg, or about 5.0×10⁶ cells/kg of enhanced APCs are administered on day 1 of each three-week cycle. In some aspects, any one of about 0.25×10⁶ cells/kg, about 0.5×10⁶ cells/kg, about 1.25×10⁶ cells/kg, about 2.5×10⁶ cells/kg, about 3.25×10⁶ cells/kg, about 5.0×10⁶ cells/kg, and about 7.50×10⁶ cells/kg of enhanced APCs are administered on day 2 of each three-week cycle. In some aspects, about 0.25×10⁶ cells/kg to about 7.5×10⁶ cells/kg of enhanced APCs are administered on day 2 of each three-week cycle. In some aspects, about 0.5×10⁶ cells/kg to about 5.0×10⁶ cells/kg of enhanced APCs are administered on day 2 of each three-week cycle. In some aspects, about 0.5×10⁶ cells/kg, about 2.5×10⁶ cells/kg, or about 5.0×10⁶ cells/kg of enhanced APCs are administered on day 2 of each three-week cycle.

In some aspects, 0.5×10⁶ cells/kg of enhanced APCs are administered on day 1 of each three-week cycle, and 0.5×10⁶ cells/kg of enhanced APCs are administered on day 2 of each three-week cycle. In some aspects, 0.5×10⁶ cells/kg of enhanced APCs are administered on day 1 of each three-week cycle, and 0.5×10⁶ cells/kg of enhanced APCs are administered on day 3 of each three-week cycle. In some aspects, 2.5×10⁶ cells/kg of enhanced APCs are administered on day 1 of each three-week cycle, and 2.5×10⁶ cells/kg of enhanced APCs are administered on day 2 of each three-week cycle. In some aspects, 2.5×10⁶ cells/kg of enhanced APCs are administered on day 1 of each three-week cycle, and 2.5×10⁶ cells/kg of enhanced APCs are administered on day 3 of each three-week cycle. In some aspects, 5.0×10⁶ cells/kg of enhanced APCs are administered on day 1 of each three-week cycle, and 5.0×10⁶ cells/kg of enhanced APCs are administered on day 2 of each three-week cycle. In some aspects, 5.0×10⁶ cells/kg of enhanced APCs are administered on day 1 of each three-week cycle, and 5.0×10⁶ cells/kg of enhanced APCs are administered on day 3 of each three-week cycle. In some aspects, 0.25×10⁶ cells/kg of enhanced APCs are administered on day 1 of each three-week cycle, and 0.25×10⁶ cells/kg of enhanced APCs are administered on day 2 of each three-week cycle. In some aspects, 0.25×10⁶ cells/kg of enhanced APCs are administered on day 1 of each three-week cycle, and 0.25×10⁶ cells/kg of enhanced APCs are administered on day 3 of each three-week cycle. In some aspects, 1.25×10⁶ cells/kg of enhanced APCs are administered on day 1 of each three-week cycle, and 1.25× 10⁶ cells/kg of enhanced APCs are administered on day 2 of each three-week cycle. In some aspects, 1.25×10⁶ cells/kg of enhanced APCs are administered on day 1 of each three-week cycle, and 1.25×10⁶ cells/kg of enhanced APCs are administered on day 3 of each three-week cycle. In some aspects, 3.25×10⁶ cells/kg of enhanced APCs are administered on day 1 of each three-week cycle, and 3.25×10⁶ cells/kg of enhanced APCs are administered on day 2 of each three-week cycle. In some aspects, 3.25×10⁶ cells/kg of enhanced APCs are administered on day 1 of each three-week cycle, and 3.25× 10⁶ cells/kg of enhanced APCs are administered on day 3 of each three-week cycle. In some aspects, 7.50×10⁶ cells/kg of enhanced APCs are administered on day 1 of each three-week cycle, and 7.50×10⁶ cells/kg of enhanced APCs are administered on day 2 of each three-week cycle. In some aspects, 7.50×10⁶ cells/kg of enhanced APCs are administered on day 1 of each three-week cycle, and 7.50×10⁶ cells/kg of enhanced APCs are administered on day 3 of each three-week cycle.

In some aspects, wherein a method further comprises administering an immune checkpoint inhibitors, the immune checkpoint inhibitor is targeted to PD-1, PD-L1, and/or CTLA-4. In some aspects, the antibody that binds PD-1, the antibody that binds PD-L1, and/or the antibody that binds CTLA-4 is administered 1, 2, 3, 4, 5, 6, 7, 8, 9, 10 or more times per cycle. In some aspects, the antibody that binds PD-1 is administered once per three-week cycle. In some aspects, the antibody that binds PD-L1 is administered once per three-week cycle. In some aspects, the antibody that binds CTLA-4 is administered once per three-week cycle. In some aspects, the antibody that binds PD-1 is administered once per two three-week cycles. In some aspects, the antibody that binds PD-L1 is administered once per two three-week cycles. In some aspects, the antibody that binds CTLA-4 is administered once per two three-week cycles.

In some aspects, according to any one of the methods described herein, the immune checkpoint inhibitor is administered in any one of a 1-, 2-, 3—, 4-, 5-, 6-, 7-, 8-, 9-, or 10-week cycle. In some aspects, the immune checkpoint inhibitor is administered on day 1 of any one of a 1-, 2-, 3—, 4-, 5-, 6-, 7-, 8-, 9-, or 10-week cycle. In some aspects, the immune checkpoint inhibitor is administered one or more times on day 1, 2, 3, 4, 5, 6, 7, 8, 9, 10, 11, 12, 13, 14, 15, 16, 17, 18, 19, or 20 in a treatment cycle.

In some aspects, the immune checkpoint inhibitor is an antibody that binds PD-1, wherein the antibody that binds PD-1 is administered on day 1 of a three-week cycle. In some aspects, the immune checkpoint inhibitor is an antibody that binds PD-1, wherein the antibody that binds PD-1 is administered on day 1 of each three-week cycle. In some aspects, the immune checkpoint inhibitor is an antibody that binds PD-1, wherein the antibody that binds PD-1 is administered on day 8 of a three-week cycle. In some aspects, the immune checkpoint inhibitor is an antibody that binds PD-1, wherein the antibody that binds PD-1 is administered on day 8 of the first three-week cycle. In some aspects, the immune checkpoint inhibitor is an antibody that binds PD-1, wherein the antibody that binds PD-1 is administered on day 8 of the first three-week cycle and day 1 of each subsequent three-week cycle. In some aspects, the immune checkpoint inhibitor is an antibody that binds PD-1, wherein the antibody that binds PD-1 is administered on day 1 of the third three-week cycle and day 1 of each subsequent three-week cycle.

In some aspects, the immune checkpoint inhibitor is an antibody that binds PD-1, wherein the antibody that binds PD-1 is pembrolizumab, wherein the pembrolizumab is administered on day 1 of a three-week cycle at a dose of about 200 mg. In some aspects, the immune checkpoint inhibitor is an antibody that binds PD-1, wherein the antibody that binds PD-1 is pembrolizumab, wherein the pembrolizumab is administered on day 1 of each three-week cycle at a dose of about 200 mg. In some aspects, the immune checkpoint inhibitor is an antibody that binds PD-1, wherein the antibody that binds PD-1 is pembrolizumab, wherein the pembrolizumab is administered on day 8 of a three-week cycle at a dose of about 200 mg. In some aspects, the immune checkpoint inhibitor is an antibody that binds PD-1, wherein the antibody that binds PD-1 is pembrolizumab, wherein the pembrolizumab is administered on day 8 of the first three-week cycle at a dose of about 200 mg. In some aspects, the immune checkpoint inhibitor is an antibody that binds PD-1, wherein the antibody that binds PD-1 is pembrolizumab, wherein the pembrolizumab is administered on day 8 of the first three-week cycle and day 1 of each subsequent three-week cycle at a dose of about 200 mg. In some aspects, the immune checkpoint inhibitor is an antibody that binds PD-1, wherein the antibody that binds PD-1 is pembrolizumab, wherein the pembrolizumab is administered on day 1 of the third three-week cycle and day 1 of each subsequent three-week cycle at a dose of about 200 mg.

In other aspects, the immune checkpoint inhibitor is an antibody that binds PD-1, wherein the antibody that binds PD-1 is nivolumab, wherein the nivolumab is administered on day 1 of a three-week cycle at a dose of about 360 mg. In some aspects, the immune checkpoint inhibitor is an antibody that binds PD-1, wherein the antibody that binds PD-1 is nivolumab, wherein the nivolumab is administered on day 1 of each three-week cycle at a dose of about 360 mg. In some aspects, the immune checkpoint inhibitor is an antibody that binds PD-1, wherein the antibody that binds PD-1 is nivolumab, wherein the nivolumab is administered on day 8 of a three-week cycle at a dose of about 360 mg. In some aspects, the immune checkpoint inhibitor is an antibody that binds PD-1, wherein the antibody that binds PD-1 is nivolumab, wherein the nivolumab is administered on day 8 of the first three-week cycle at a dose of about 360 mg. In some aspects, the immune checkpoint inhibitor is an antibody that binds PD-1, wherein the antibody that binds PD-1 is nivolumab, wherein the nivolumab is administered on day 8 of the first three-week cycle and day 1 of each subsequent three-week cycle at a dose of about 360 mg. In some aspects, the immune checkpoint inhibitor is an antibody that binds PD-1, wherein the antibody that binds PD-1 is nivolumab, wherein the nivolumab is administered on day 1 of the third three-week cycle and day 1 of each subsequent three-week cycle at a dose of about 360 mg.

In some aspects, the immune checkpoint inhibitor is an antibody that binds PD-L1, wherein the antibody that binds PD-L1 is administered on day 1 of a three-week cycle. In some aspects, the immune checkpoint inhibitor is an antibody that binds PD-L1, wherein the antibody that binds PD-L1 is administered on day 1 of each three-week cycle. In some aspects, the immune checkpoint inhibitor is an antibody that binds PD-L1, wherein the antibody that binds PD-L1 is administered on day 8 of the first three-week cycle and on day 1 of each subsequent cycle. In some aspects, the immune checkpoint inhibitor is an antibody that binds PD-L1, wherein the antibody that binds PD-L1 is atezolizumab, wherein the atezolizumab is administered on day 1 of a three-week cycle at a dose of about 1200 mg. In some aspects, the immune checkpoint inhibitor is an antibody that binds PD-L1, wherein the antibody that binds PD-L1 is atezolizumab, wherein the atezolizumab is administered on day 1 of each three-week cycle at a dose of about 1200 mg. In some aspects, the immune checkpoint inhibitor is an antibody that binds PD-L1, wherein the antibody that binds PD-L1 is atezolizumab, wherein the atezolizumab is administered on day 8 of the first three-week cycle and on day 1 of each subsequent cycle at a dose of about 1200 mg.

In some aspects, the immune checkpoint inhibitor is an antibody that binds CTLA-4, wherein the antibody that binds CTLA-4 is administered on day 1 of a three-week cycle. In some aspects, the immune checkpoint inhibitor is an antibody that binds CTLA-4, wherein the antibody that binds CTLA-4 is administered on day 1 of each three-week cycle. In some aspects, the immune checkpoint inhibitor is an antibody that binds CTLA-4, wherein the antibody that binds CTLA-4 is administered for a maximum of four doses. In some aspects, the immune checkpoint inhibitor is an antibody that binds CTLA-4, wherein the antibody that binds CTLA-4 is administered once per two three-week cycles. In some aspects, the immune checkpoint inhibitor is an antibody that binds CTLA-4, wherein the antibody that binds CTLA-4 is ipilimumab, wherein the ipilimumab is administered on day 1 of a three-week cycle at a dose of about 3 mg/kg. In some aspects, the immune checkpoint inhibitor is an antibody that binds CTLA-4, wherein the antibody that binds CTLA-4 is ipilimumab, wherein the ipilimumab is administered on day 1 of each three-week cycle at a dose of about 3 mg/kg. In some aspects, the immune checkpoint inhibitor is an antibody that binds CTLA-4, wherein the antibody that binds CTLA-4 is ipilimumab, wherein the ipilimumab is administered for a maximum of four doses at a dose of about 3 mg/kg. In some aspects, the immune checkpoint inhibitor is an antibody that binds CTLA-4, wherein the antibody that binds CTLA-4 is ipilimumab, wherein the ipilimumab is administered once per two three-week cycles at a dose of about 3 mg/kg.

### Composition of Enhanced APCs

In some aspects, according to methods described herein, the composition comprising enhanced APCs comprises about 1.0×10⁶ to about 1.0×10⁸ enhanced APCs/mL. In some aspects, the composition comprising enhanced APCs comprises any one of about 1.0×10⁶ to about 0.5×10⁷, about 0.5×10⁷ to about 1.0×10⁷, about 1.0×10⁷ to about 0.5×10⁸, and about 0.5×10⁸ to about 1.0×10⁸ enhanced APCs/mL. In some aspects, the composition comprising enhanced APCs comprises about any one of about 1.0×10⁶, about 0.2×10⁷, about 0.3×10⁷, about 0.4×10⁷, about 0.5×10⁷, about 0.6×10⁷, about 0.7×10⁷, about 0.8×10⁷, about 0.9×10⁷, about 1.0×10⁷, about 0.2×10⁸, about 0.3×10⁸, about 0.4×10⁸, about 0.5×10⁸, about 0.6×10⁸, about 0.7×10⁸, about 0.8×10⁸, about 0.9×10⁸, and about 1.0×10⁸ enhanced APCs/mL. In some aspects, the composition comprising enhanced APCs comprises about 8.5×10⁶ enhanced APCs/mL. In other aspects, the composition comprising enhanced APCs comprises about 1.1×10⁷ enhanced APCs/mL.

In some aspects, the composition comprises about 5.0×10⁶ to about 1.0×10⁹ enhanced APCs. In some aspects, the composition comprises any one of about 5.0×10⁶ to about 1.0×10⁷, about 1.0×10⁷ to about 0.5×10⁸, about 0.5×10⁸ to about 1.0×10⁸, about 1.0×10⁸ to about 0.5×10⁹, and about 0.5×10⁹ to about 1.0×10⁹ enhanced APCs. In some aspects, the composition comprises any one of about 5.0×10⁶, about 6.0×10⁶, about 7.0×10⁶, about 8.0×10⁶, about 9.0×10⁶, about 1.0×10⁷, about 2.0×10⁷, about 3.0×10⁷, about 4.0×10⁷, about 5.0×10⁷, about 6.0×10⁷, about 7.0×10⁷, about 8.0×10⁷, about 9.0×10⁷, about 1.0×10⁸, about 2.0×10⁸, about 3.0×10⁸, about 4.0×10⁸, about 5.0×10⁸, about 6.0×10⁸, about 7.0×10⁸, about 8.0×10⁸, about 9.0×10⁸, and about 1.0×10⁹ enhanced APCs. In some aspects, the composition comprises about 8.1×10⁷ enhanced APCs. In other aspects, the composition comprises about 1.05×10⁸ enhanced APCs.

In some aspects, according to methods described herein, the composition comprising enhanced APCs comprises a cryopreservation medium. In some aspects, the composition comprising enhanced APCs comprises cryopreservation medium at a concentration of about any one of 20%, 25%, 30%, 35%, 40%, 45%, 50%, 55%, 60%, 65%, 70%, 75%, 80%, or 85% (w/w). In some aspects, the composition comprising enhanced APCs comprises cryopreservation medium at a concentration of about any one of 20% to 25%, 25% to 30%, 30% to 35%, 35% to 40%, 40% to 45%, 45% to 50%, 50% to 55%, 55% to 60%, 60% to 65%, 65% to 70%, 70% to 75%, 75% to 80% or 80% to 85% (w/w).

In some aspects, according to methods described herein, the composition comprising enhanced APCs comprises a hypothermic preservation medium. In some aspects, the composition comprising enhanced APCs comprises hypothermic preservation medium at a concentration of about any one of 10%, 15%, 20%, 25%, 30%, 35%, 40%, 45%, 50%, 55%, 60%, 65%, or 70% (w/w). In some aspects, the composition comprising enhanced APCs comprises hypothermic preservation medium at a concentration of about any one of 10% to 15%, 15% to 20%, 20% to 25%, 25% to 30%, 30% to 35%, 35% to 40%, 40% to 45%, 45% to 50%, 50% to 55%, 55% to 60%, 60% to 65%, or 65% to 70% (w/w).

In some aspects, according to methods described herein, the composition comprising enhanced APCs comprises human serum albumin at a concentration of about any one of 2%, 3%, 4%, 5%, 8%, or 10% (w/w). In some aspects, the composition comprising enhanced APCs comprises human serum albumin at a concentration of about any one of 2% to 3%, 3% to 5%, 5% to 8%, or 8% to 10% (w/w). In some aspects, human serum albumin is added to the composition as a human serum albumin solution. In some aspects, the concentration of the human serum albumin solution in the composition is about any one of 15%, 20%, 25%, 30%, 35%, 40%, 45%, or 50% (w/w). In some aspects, the concentration of the human serum albumin solution in the composition is about any of 10% to 15%, 15% to 20%, 20% to 25%, 25% to 30%, 30% to 35%, 35% to 40%, 40% to 45%, or 45% to 50% (w/w).

In some aspects, according to methods described herein, the pH of the composition is about 5.0 to about 9.5. In some aspects, the pH of the composition is about 6.0 to about 8.5. In some aspects, the pH of the composition is about 7.0 to about pH 7.9. In some aspects, the pH of the composition is about 7.4. In some aspects, the pH of the composition is any one of about 5, 5.5, 6, 6.5, 7, 7.5, 8, 8.5, 9, or 9.5. In other aspects, the pH of the composition is any one of about 7, 7.1, 7.2, 7.3, 7.4, 7.5, 7.6, 7.7, 7.8, or 7.9. In some aspects, the pH of the composition is any one of about 5 to about 6, about 6 to about 7, about 7 to about 8, about 8 to about 9, or about 9 to about 10. In some aspects, the pH of the composition is any one of about 7 to about 7.1, about 7.1 to about 7.2, about 7.2 to about 7.3, about 7.3 to about 7.4, about 7.4 to about 7.5, about 7.5 to about 7.6, about 7.6 to about 7.7, about 7.7 to about 7.8, or about 7.8 to about 7.9.

In some aspects, the cryopreservation medium comprises CryoStor® CS10. In some aspects, the composition comprising enhanced APCs comprises about 5.0×10⁶ to about 1.0×10⁹ enhanced APCs in CryoStor® CS10. In some aspects, the composition comprising enhanced APCs comprises about 5×10⁶ to about 5×10⁷ enhanced APCs in CryoStor® CS10.

In some aspects, the composition comprising enhanced APCs comprises: a) about 5×10⁶ enhanced APCs to about 1×10⁹ enhanced APCs; b) cryopreservation medium at a concentration of about 40% to about 95% (w/w); c) hypothermic preservation medium at a concentration of about 25% to about 35% (w/w); and d) human serum albumin solution at a concentration of 15% to about 25% (w/w), wherein the pH of the composition is about pH 6.0 to about pH 8.5.

In some aspects, the composition comprising enhanced APCs comprises: a) about 8.1×10⁷ enhanced APCs; b) cryopreservation medium at a concentration of about 50% (w/w); c) hypothermic preservation medium at a concentration of about 30% (w/w); and d) human serum albumin solution at a concentration of 20% (w/w), wherein the pH of the composition is about pH 7.0 to about pH 7.9.

In other aspects, the composition comprising enhanced APCs comprises: a) about 1.05×10⁸ enhanced APCs; b) cryopreservation medium at a concentration of about 50% (w/w); c) hypothermic preservation medium at a concentration of about 30% (w/w); and d) human serum albumin solution at a concentration of 20% (w/w), wherein the pH of the composition is about pH 7.0 to about pH 7.9.

In some aspects, provided herein is a method for treating a human papilloma virus (HPV)-associated cancer in a subject in need thereof, the method comprising administering to the subject: (i) a composition comprising modified antigen presenting cells (“enhanced APCs”), wherein the enhanced APCs comprise an HPV E6 and/or HPV E7 antigen, CD86, membrane-bound interleukin-2 (“mbIL-2”), and membrane-bound interleukin-12 (“mbIL-12”), wherein the enhanced APCs are capable of activating T cells in an HLA agnostic manner, and (ii) an antagonist of PD-1.

In some aspects, provided herein is a method for treating a HPV+ recurrent, locally advanced, or metastatic tumor in a subject in need thereof, the method comprising administering a composition comprising modified antigen presenting cells (“enhanced APCs”), wherein the enhanced APCs comprise an HPV E6 and/or HPV E7 antigen, CD86, membrane-bound interleukin-2 (“mbIL-2”), and membrane-bound interleukin-12 (“mbIL-12”), and wherein the enhanced APCs are capable of activating T cells in an HLA agnostic manner.

### EXAMPLES

Those skilled in the art will recognize that several aspects are possible within the scope and spirit of this disclosure. The disclosure will now be described in greater detail by reference to the following non-limiting examples. The following examples further illustrate the disclosure but, of course, should not be construed in any way limits its scope.

### Example 1. Clinical Protocol

A Phase 1/2, first-in-human, multicenter, open-label study of the use of enhanced APCs described herein as a monotherapy and in combination with immune checkpoint inhibitor in patients with HPV16+ recurrent, locally advanced, or metastatic solid tumors is conducted.

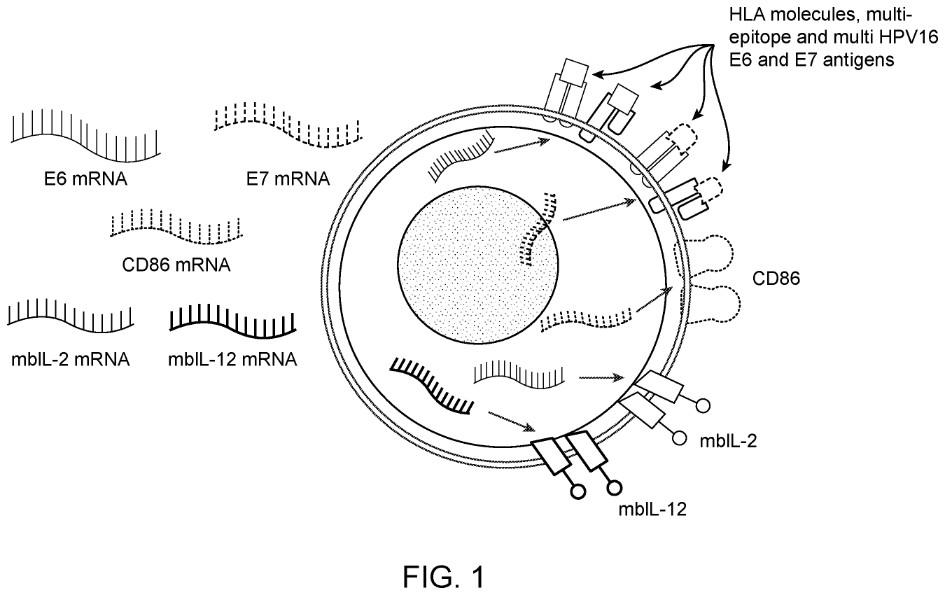

As described herein, the enhanced APCs for the above trial have been produced by squeeze delivering into PBMCs the following nucleic acid constructs: (1) mRNA encoding the full-length of HPV-16 E6 protein (SEQ ID NO: 14), (2) mRNA encoding the full-length of HPV-16 E7 protein (SEQ ID NO: 17), (3) mRNA encoding CD86, (4) mRNA encoding membrane bound IL-2, and mRNA encoding membrane-boudn IL-12. (See FIG. 1). Prior to use, the enhanced APCs have been formulated in a solution containing 50% (w/w) of CryoStor® CS10, 30% (w/w) of HypoThermosol® FRS, and 20% (w/w) of 25% Human Serum Albumin (HSA) to a target concentration of 11×10⁶ live cells/mL, and filling into vials. The pH of the formulation was adjusted to 7.0-7.9. The vials were cryopreserved using a chamber temperature of ≤−170° C., with the vials reaching and maintaining a temperature of ≤−140° C. during the different stages of the development process (e.g., production and storage). Unless indicated otherwise, the term “drug substance” is used herein to refer to the enhanced APCs of the present disclosure. Unless indicated otherwise, the term “drug product” refers to the formulation comprising the drug substance which is ultimately administed to the subjects to be treated.

### Overview

The study population consists of adult patients with HPV16+ recurrent, locally advanced, or metastatic solid tumors (head and neck, cervical, anal, vulvar, penile), who have met key eligibility criteria, and in whom the HPV16+tumor status has been diagnosed via local standard of care (SOC) methods tumors. HPV16+status can be confirmed by a local report reviewed by the Sponsor. If the locally obtained documentation establishing HPV16+tumor status is deemed insufficient by the Sponsor, central confirmation should be done using whole blood for circulating HPV DNA analysis, or from archival tumor tissue, or a fresh tumor biopsy collected at Screening.

Alternatively, whole blood for tumor tissue modified virus (TTMV) HPV16 (cfDNA) or tumor tissue (archival or fresh) can be provided for central laboratory analysis. Screening should be completed within 28 days, 7-14 days prior to leukapheresis.

Eligible patients must undergo leukapheresis. The leukapheresis product is sent to the manufacturer to produce each patient's personalized autologous cellular therapy. Frozen vials of drug product will then be sent to the study sites for administration.

This study is conducted in 2 parts. The initial parts Part 1 (1A and 1B) will determine the recommended phase 2 dose (RP2D) of the drug product (as monotherapy) and evaluate the safety and preliminary efficacy when combined with pembrolizumab in patients with HPV+solid tumors who have progressed from available therapies, including ICIs. In Part 1A, the monotherapy is established through dose escalation cohorts in a BOIN design. In Part 1B, the safety and preliminary efficacy of the drug product is further assessed in combination with pembrolizumab at RP2D or a calibrated dose level based on overall safety data. Patients receive the drug product at a dose level of RP2D or lower, and the standard dose (200 mg) of pembrolizumab.

Part 2 initiates ICI therapy naïve patients with HPV16+ recurrent, locally advanced, or metastatic tumors who have progressed after standard of care treatment. Patients are ICI therapy naïve patients when diagnosed with HPV16+solid tumors and who have progressed from standard of care (SOC) but have not been treated with a PD-1, PD-L1 or CTLA-4 inhibitor, or any other agent targeting T-cell co-stimulation or checkpoint pathways. The goal of combining the drug product and pembrolizumab in a lead-in strategy is to change the tumor microenvironment by increasing the amount of tumor infiltrating lymphocytes that have been associated with improved clinical response to immunotherapy with checkpoint inhibitors. The patients receive the drug product at RP2D (from Part 1) and a standard dose (200 mg) of pembrolizumab administered after 6 weeks of the drug product monotherapy lead-in.

Each art of the study is to utilize the Bayesian optimal interval (BOIN) design, used to find the maximum tolerated dose (MTD) with a target DLT rate of 30% for the drug product. Following the BOIN design, a minimum of 3 (unless both of the first 2 patients have DLT) and up to 12 patients are enrolled in each monotherapy cohort, and a dose level can be declared safe when ≤30% patients experience a DLT. Should the toxicity level be higher than 30% DLTs, the dose can be deescalated. If there is lack of immunogenic effect and no safety signal, the cohort can be declared safe. The choice to expand beyond 6 patients in a cohort can be made to further investigate safety and tolerability, immunogenic effects, and antitumor activity at dose levels likely to be the RP2D. The final sample size depends on the dosing schedule, the number of DLTs, and total number of treatment cohorts for determining the RP2D. Up to approximately 60 safety-evaluable patients can be enrolled.

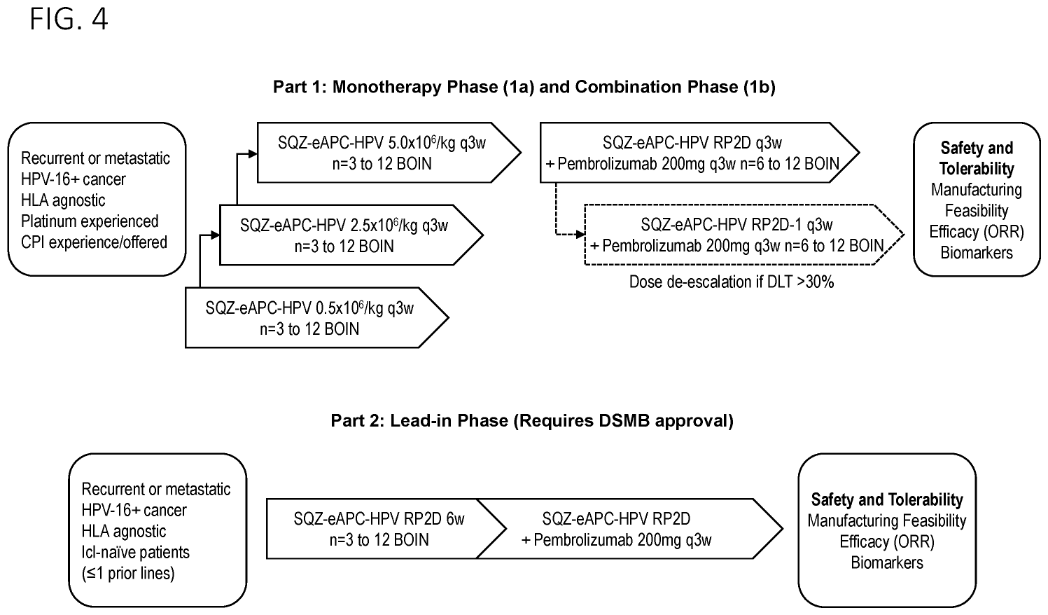

Prior to the start of study treatment, eligible patients undergo a leukapheresis at the study sites. The leukopak are sent to the manufacturer to generate each patient's personalized autologous cellular therapy. Frozen vials of the drug product are then to be sent to the study sites for administration. The study schema is shown as FIG. 4.

### Part 1A: Dose Escalation (Monotherapy)

To determine the safety profile, preliminary efficacy, and RP2D of the drug product as a monotherapy, 3 dose levels of the drug product are to be evaluated as per the following cohorts shown in Table 1.

While the traditional 3+3 design is intended to assess safety and tolerability, it can be prudent to treat up to 6 additional patients in a cohort to further investigate safety and tolerability, immunogenic effects, and antitumor activity. There is a maximum of 12 patients per cohort in this modified 3+3 design. There must be a minimum of 6 patients.

TABLE 1
Summary of Monotherapy Cohorts Planned
During the Escalation Phase

| | Dose of Drug Product | |
|---|---|---|
| Cohort | (cells/kg) | Total Doses^{a} |
| 1 | 0.5 × 10⁶ | ≥3 |
| 2 | 2.5 × 10⁶ | ≥3 |
| 3 | 5.0 × 10⁶ | ≥3 |

DLT = dose-limiting toxicity;
iRECIST = modified RECIST criteria for incorporation into solid tumor studies of immunotherapeutics;
RECIST = Response Evaluation Criteria for Solid Tumours version 1.1
^{a}Dosing with drug product continues every 3 weeks until treatment discontinuation criteria are met, or until the drug product supply is exhausted, or for a maximum of 1 year, whatever comes first. Patients who experience disease progression per RECIST 1.1 can continue dosing if considered in their best interest by the treating Investigator to allow for confirmation of disease progression; ie, iCPD according to iRECIST (Seymour et al. 2017).
b. In Cycle 1, patients receive drug product on Day 1.

Following the Safety Study Committee's (SSC's) review of the safety, efficacy, and pharmacodynamic data from patients in the available cohorts, the SSC can determine that the assessment of dose levels higher than 5.0×10⁶ mbIL-2 in CD45+ cells/kg body weight or lower than 0.5×10⁶ mbIL-2 in CD45+ cells/kg body weight is warranted. If the SSC deems it necessary based on review of available safety data, the following provisional cohorts can be opened prior to opening Part 1B. See Table 2.

TABLE 2
Expanded Summary of Monotherapy Cohorts
Planned During the Escalation Phase

| | Dose of Drug Product | |
|---|---|---|
| Cohort | (cells/kg) | Total Doses^{a} |
| −1 | 0.25 × 10⁶ | ≥3 |
| 1P | 1.25 × 10⁶ | ≥3 |
| 2P | 3.25 × 10⁶ | ≥3 |
| 3P | 7.5 × 10⁶ | ≥3 |

The SSC can decide to open Part 1B without opening any of the provisional cohorts noted above. The cohort of the dose selected for RP2D by the SSC must have a minimum of 6 patients treated with the drug product as a monotherapy.

Patients must have sufficient autologous drug product to achieve at least 3 full dose administrations of the drug product to be dosed in the currently enrolling cohort; otherwise, they are assigned to a lower dose cohort. Should the toxicity level be higher than 30% DLTs, the dose can be de-escalated. Patients are evaluated in accordance with the BOIN design and will accrue to the study in cohorts of a minimum of 3 patients, up to 12 patients at each dose level. Additional patients (up to a total of 12) can be treated in an optional exploratory cohort to further investigate immunogenic effects and antitumor activity of the drug product as a monotherapy.

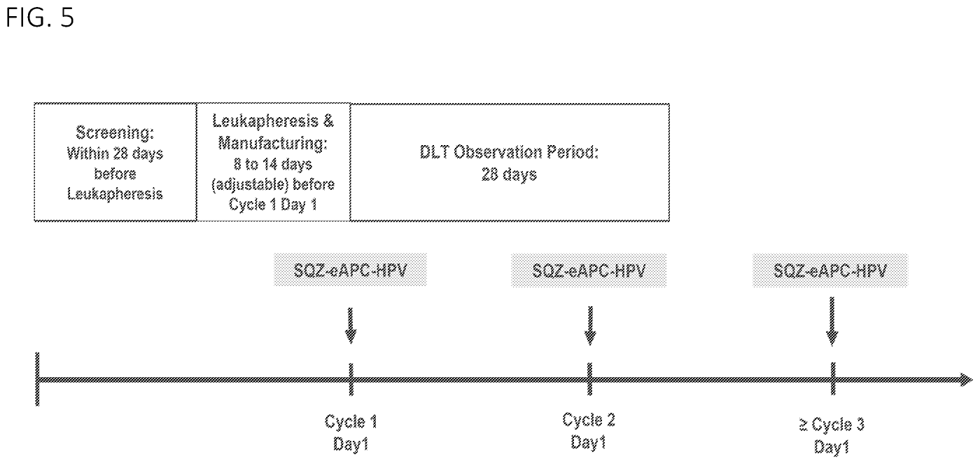

As shown in FIG. 5, patients are administered the drug product as a monotherapy on Day 1 of each 3-week treatment cycle (ie, C1D1, C2D1, C3D1, etc.) for a maximum of 1 year, or until the drug product supply is exhausted or treatment discontinuation criteria are met, whichever occurs first. All adverse events of special interest (AESIs) should resolve to ≤Grade 1 prior to the start of a new cycle of treatment. Other study drug-related adverse events (AEs) should resolve to Grade ≤2 prior to the start of new cycle of treatment. Patients are monitored for the occurrence of DLTs for 28 days after the first dose of the drug product. To be evaluable for DLT assessment, patients must complete the 28-day safety observation period (unless DLT has occurred within the observed period). Patients who are considered non-evaluable for DLT assessment are replaced. Patients who experience disease progression per the Response Evaluation Criteria for Solid Tumors version 1.1 (RECIST 1.1) can continue dosing if considered in their best interest by the treating Investigator to allow for confirmation of disease progression. The first 2 patients in each cohort undergo a minimum 23 hours of observation after the first administration of the drug product.

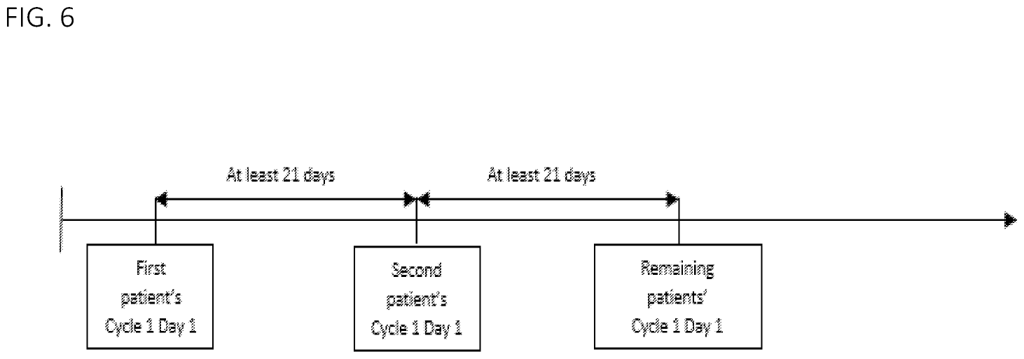

As shown in FIG. 6, the enrollment of the first 2 patients are staggered with a 21-day interval between each patient. As an extra safety measure, all patients in the study is observed for at least 4 hours after each administration of the drug product.

Part 1: Escalation Phase (Drug Product in Combination with Immune Checkpoint Inhibitor)

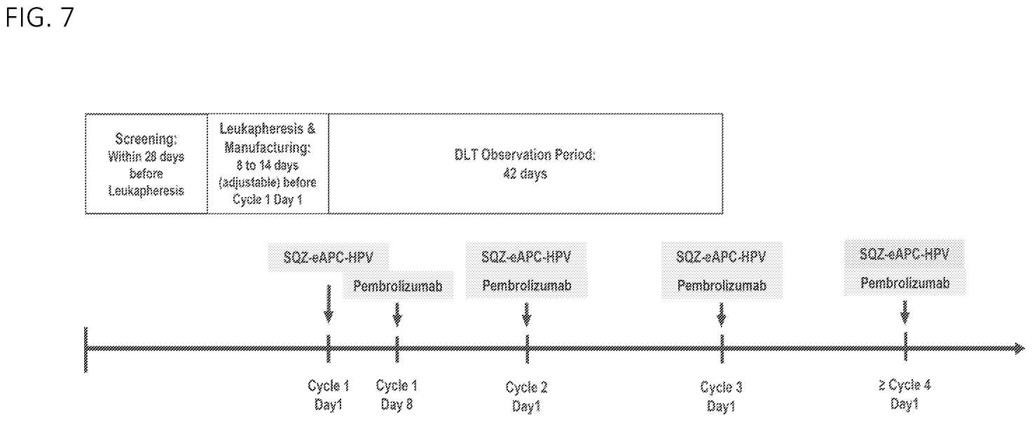

To assess the safety profile and preliminary efficacy of the drug product when combined with pembrolizumab, a minimum of 3, to a maximum of 12 patients are enrolled and evaluated in accordance with the BOIN design. Patients will receive the drug product at a dose level of RP2D or lower, as defined by the SSC and the Sponsor, on Day 1 of Cycle 1 (C1D1), and standard dose (200 mg) of pembrolizumab on Day 8 of Cycle 1 (C1D8). In subsequent cycles, patients are first administered the drug product, and then the standard dose of pembrolizumab on Day 1 of each cycle (C2D1, C3D1.etc.). Patients are treated in a 21-days treatment cycle for a maximum of 1 year with the drug product or until the autologous drug product is exhausted, and 2 years with pembrolizumab, or until the treatment discontinuation criteria are met, whichever comes first, as illustrated in FIG. 7. The first 2 patients in each cohort will undergo a minimum 23 hours of observation after the first administration of the drug product. The enrollment of the first 2 patients are staggered with a 21-day interval between each patient. As an extra safety measure, all patients in the study are observed for at least 4 hours after each administration of the drug product.

If a lower starting dose of the drug product is selected, 3 patients are enrolled initially, and treated with the drug product and pembrolizumab. These initial 3 patients are evaluated for safety for 42 days before enrolling additional patients. If none of the first 3 (0/3) patients experience a DLT during the 42-day safety evaluation period, then the drug product dose can be escalated to the RP2D as defined in Part 1A. If a DLT is observed in 1 out of the first 3 (1/3) patients, additional patients (up to a total of 12) are accrued using a BOIN design, with a safety evaluation period of 42 days.

A minimum of 6 patients are required to determine final RP2D for use in Part 1B and are based on review of available safety, tolerability, immunogenic, and other pharmacodynamic and antitumor data.

Patients who experience disease progression per Response Evaluation Criteria for Solid Tumors version 1.1 (RECIST 1.1) can continue dosing if considered in their best interest by the treating Investigator to allow for confirmation of disease progression.

Patients in Part 1A and 1B are enrolled in staggered manner in all cohorts (ie, the first 2 patients are enrolled no less than 21 days apart in each cohort). All AESIs should resolve to Grade ≤1 prior to the start of a new cycle of treatment. Other study drug-related AEs should resolve to Grade ≤2 prior to the start of new cycle of treatment.

Part 2: Lead-in Drug Product in Combination with Immune Checkpoint Inhibitor

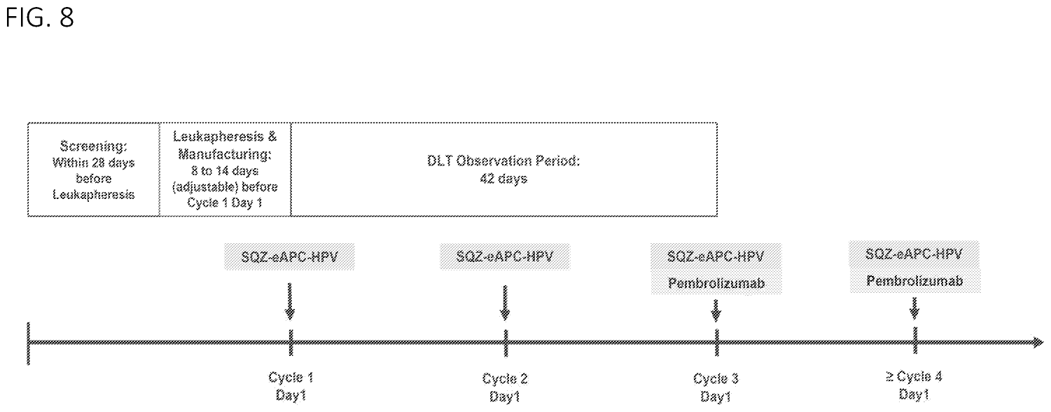

To further assess the safety and preliminary efficacy of the drug product in combination with pembrolizumab, ICI naïve patients with HPV16+solid tumors will receive the drug product as a monotherapy at the RP2D (from Part 1B) on Day 1 of each treatment cycle. Treatment with pembrolizumab will commence in Cycle 3. Starting from Cycle 3, and in all subsequent cycles, standard doses of pembrolizumab are administered on day 1 after the drug product administration, as shown in FIG. 8. Patients are treated in 21-days treatment cycles for a maximum of 1 year with the drug product or until the autologous drug product is exhausted, and 2 years with pembrolizumab, or until the treatment discontinuation criteria are met, whichever occurs first. A BOIN design is employed for accrual and evaluation of patients. All AESIs should resolve to Grade ≤1 prior to the start of a new cycle of treatment. Other study drug-related AEs should resolve to Grade ≤2 prior to the start of new cycle of treatment.

If DLT is observed in 1 out of the first 3 (1/3) patients, additional (up to a total of 12) patients will be accrued using a BOIN design, with a safety evaluation period of 42 days. If at any point, the DLT rate is found to be ≥30%, all available data for aggregate safety, pharmacodynamics, and efficacy data will be reviewed. Based on review of the data, it can be determined that a lower dose level of the drug product should be explored (dose de-escalation) or it can be decided to stop enrollment altogether.

Patients who experience disease progression per RECIST 1.1 can continue dosing if considered in their best interest by the treating Investigator to allow for confirmation of disease progression.

### Dose-Limiting Toxicity

A patient is considered evaluable for DLT assessment if they: a) experience a DLT during the DLT assessment period (28 days in Part 1A, 42 days in Part 1B and Part 2), regardless of the dose administered; or b) do not experience a DLT during the DLT assessment period after having received 2 doses of the drug product (Part 1A), and those who complete the drug product monotherapy lead-in period of 42 days and at least 1 dose of Pembrolizumab (Parts 1B and 2).

The minimum number of mbIL-2 in CD45+ cells per dose needed to be considered evaluable for DLTs will be 70% of the target dose. Patients with a manufactured cell dose of less than the 70% of target dose will be excluded from DLT assessment. However, these patients will be included in safety, dose feasibility, and exploratory studies.

Patients experiencing a DLT that is not an IRR are discontinued from the study. If it is in the patient's best interest to continue treatment on investigational product, then the subsequent treatment will be determined by the Investigator in consultation with the Sponsor. For IRRs, the premedication or rate of administration is adjusted to enable the patient to remain on study. Patients who experience Grade 3 IRRs can remain on study treatment if the event resolves to Grade ≤2 within 72 hours. The premedication or rate of administration can be adjusted to enable the patient to remain on study.

A DLT is defined as an AE or abnormal laboratory value assessed by the Principal Investigator and confirmed by the SSC as unrelated to disease, disease progression, intercurrent illness, concomitant medications/procedures, or environmental factors, but related to the drug product (either alone or in combination), occurring within either the first 28 days of treatment with monotherapy or the first 42 days of treatment with combination therapy, and which meets any of the pre-defined criteria as listed below using National Cancer Institute (NCI) Common Terminology Criteria for Adverse Events (CTCAE) version 5.0. Grading of CRS and neurotoxicity will use the American Society for Transplantation and Cellular Therapy (ASTCT) Consensus Grading.

### Non-Hematologic Toxicity

Grade 4 or Grade 5
Grade 3 toxicity that does not resolve to Grade <2 or Baseline within 7 days despite optimal supportive care, except for Grade 3 CRS or neurotoxicity that does not resolve to Grade ≤2 within 24 hours
Grade 3 autoimmune or hypersensitivity reaction
Grade 3 infusion related reactions that do not resolve to Grade ≤2 within 72 hours Grade ≥3 hepatic toxicity lasting for >48 hours with the following exception: for patients with Grade 2 aspartate aminotransferase (AST), alanine aminotransferase (ALT), and/or alkaline phosphatase abnormalities at Baseline, only an increase to >8×ULN lasting >48 hours will be considered a DLT.
Grade 3 laboratory value that persists >7 days and requires medical intervention Liver test abnormalities meeting Hy's law criteria

### Hematologic Toxicity

Any Grade 5 toxicity
Any Grade 4 anemia
Any Grade ≥3 febrile neutropenia
Grade ≥4 neutropenia (absolute neutrophil count <500/μL) lasting >7 days
Grade ≥4 thrombocytopenia (<25,000/μL)
Grade ≥3 thrombocytopenia (<50,000/μL) lasting >7 days associated with clinically significant bleeding
Adverse Events at least possibly related to drug product (either alone or in combination) that result in permanent discontinuation or a delay of >14 days of Cycle 2 Day 1 of scheduled drug product administration
Any other event that, in the judgment of the Investigator and Sponsor, is considered to be a DLT.

The following events are not considered a DLT:
Isolated Grade 3 lipase values that are not accompanied by >Grade 3 amylase values or clinical symptoms or radiographic evidence of pancreatitis Grade 3 CRS that improves to ≤Grade 2 within 24 hours with or without symptomatic treatment
Grade 3 skin rash that resolves to ≤Grade 2 within 7 days with or without appropriate supportive care
Immediate hypersensitivity reactions occurring within 2 hours of cell product administration that are reversible to <Grade 2 with 24 hours
Grade 1 or Grade 2 electrolyte abnormalities that are corrected within 72 hours without clinical sequelae

### Alopecia

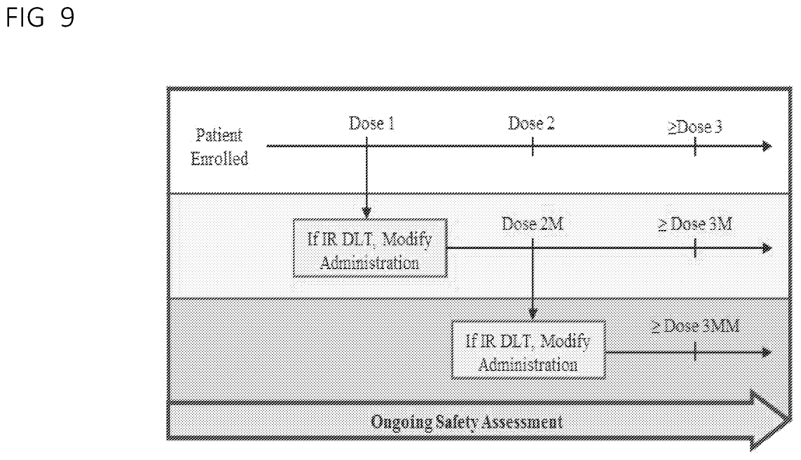

A Grade 3 IRR that can be adequately managed with the addition of premedication or modification of the rate of administration will not be considered a DLT, unless these changes are considered applicable to all subsequent patients enrolled in the study based on the recommendations of the SSC, as shown in FIG. 9. If the modification(s) applies to all subsequent patients, the cohort will restart for the DLT evaluation. The patient who experienced the Grade 3 IRR can remain in the study with modification to their premedication or the rate of administration only if the Grade 3 IRR resolved to Grade ≤2 within 72 hours.

Incidence of late-occurring, immune-mediated toxicity will be taken into consideration when determining the RP2D of drug product, either as monotherapy or in combination with ICI. If the MTD is not reached in any cohort, Cohort 3P can be tested. In the event of AEs covered by the definition of a DLT but unrelated to drug product, the findings will be discussed by the SSC.

### Stopping Criteria for a Cohort and Stopping of Dose Escalation or Progression to Cohort and Termination of Study

The BOIN design will drive the decision to declare a cohort as safe (Section 3.1.1). The minimum number of patients needed to confirm a cohort as safe is 3 patients with 0 DLTs. A cohort enrolling up to 12 patients will be confirmed safe with≤30% of patients with a DLT (for instance, 6 patients with <2 DLTs, 9 patients with <3 DLTs, or 12 patients with <4 DLTs).

An AE that meets the definition of a DLT and occurring outside the DLT window will not be counted as a DLT but instead will be considered in the overall safety assessment of a given cohort and the selection of an RP2D regimen.

The cohort stopping rule is based on being above the threshold for DLTs at a given sample size as per the following. If the stopping rule is triggered, the SSC can declare the prior tolerated dose level as the MTD; recommend testing of an intermediate dose level; discontinue enrollment and/or the study; and/or recommend a protocol amendment. Following review by the SSC, dosing of patients can be stopped based on these general safety related criteria:

Any SAE that is considered potentially life-threatening and is assessed by the Medical Monitor as related to investigational product

Any other clinically significant change that indicates to the Investigator or Sponsor a major safety risk

### Dosing Schedule and Study Duration

All patients undergo a single leukapheresis prior to the start of treatment. Patients undergo leukapheresis at study sites, typically 8 to 14 days prior to the initial administration of drug product. Scheduling of the first administration of drug product takes into account site location and shipping logistics.

A cycle is defined as a treatment period of 21 days.

The duration of treatment for the monotherapy dose escalation (Part 1A) is dependent on the selected RP2D regimen. For some patients in Part 2, the kinetics of tumor growth can initially outpace anti-tumor immune activity. With sufficient time, the anti-tumor activity will dominate and become clinically apparent. Thus, it is important to assess RECIST v.1.1 and iRECIST42 in parallel at each time point. Therefore, patients can continue study treatment after the initial RECIST v.1.1-defined progression, and therefore allow for confirmation of disease progression according to iRECIST, if the following criteria are met:
Investigator-assessed clinical benefit, and absence of rapid disease progression Tolerance of study drug as defined by the Investigator
Stable performance status
Treatment beyond progression will not impede prevention of serious consequence from rapidly progressing disease
Lack of complications of disease progression (eg, central nervous system [CNS] metastases)

The assessment of clinical benefit should take into account whether the subject is clinically deteriorating and unlikely to receive further benefit from continued treatment.

The safety observation period corresponds to the DLT (28 days for drug product monotherapy, and 42 days for combination).

After completion of the safety follow up visit (EODW6), patients will enter the long-term follow-up (LTFU) and overall survival (OS) period. Patients experiencing ongoing AEs related to study treatment should continue to be followed as clinically indicated until AEs resolve to Grade 1 or Baseline or are deemed irreversible. Patients who complete treatment or discontinue study treatment for reasons other than progressive disease, will continue to undergo tumor assessment as per the protocol specified schedule until progressive disease is documented, or subsequent treatment initiated. For OS assessment, patients will be contacted (by phone) every 3 months for up to 2 years or until death, withdrawal of consent, lost to follow-up, start of other anticancer therapy, or end of the study, whichever occurs first.

### Study Population

The study population will consist of adult patients with HPV16+solid tumors (head and neck, cervical, anal, vulval, penile), who are eligible, and in whom the HPV16+tumor status has been diagnosed via local standard of care (SOC) methods (with documentation provided to the Sponsor for confirmation). If the locally obtained documentation establishing HPV16+tumor status is deemed insufficient by the Sponsor, central confirmation can be done using tissue from a fresh (or archived) tumor biopsy or from whole blood circulating cell-free (cf) DNA analysis (liquid biopsy) collected at Screening. Eligibility criteria must be met prior to the leukapheresis for manufacture of autologous drug product.

### Number of Patients

The number of patients will depend on safety and observed immunogenic effects. Up to 60 In the Monotherapy Escalation Phase (Part 1A), it is anticipated that between 9 and 36 evaluable patients will be enrolled. If none of the planned cohorts indicate that the MTD has been reached, additional dose levels or regimens can be tested per the SSC. In the Combination Safety Phase (Part 1B), up to a total of 12 evaluable patients will be enrolled, and in drug product monotherapy lead-in combination therapy (Part 2), up to a total of 12 patients will be enrolled.

### Inclusion Criteria

1. Male or female patients ≥18 years of age

2. Be willing and capable of providing signed and dated ICF, agreeing to adhere to treatment, study procedures, and visit schedules as per protocol.

3. Histologically confirmed incurable or metastatic solid tumor (including but not limited to cervical and head and neck tumors) that are HPV16* as determined by histological or HPV16 molecular analysis.

4. Agree to venous access for the leukapheresis for manufacture of autologous blood product and be willing to have a central line inserted if venous access is an issue.

5. Have a lesion (unresectable or metastatic solid tumor) that can be biopsied with acceptable clinical risk and agree to have a fresh biopsy at Screening and on Cycle 2 Day 8 (±2 days). Every effort should be made to obtain the second biopsy from the same lesion of the biopsy at Screening. A lesion in a previously irradiated area could be biopsied as long as there is objective evidence of progression of the lesion before study enrollment.

6. At least 1 measurable lesion according to RECIST 1.1. Lesions biopsied for enrollment should not be followed for radiographic response.

7 Eastern Cooperative Oncology Group (ECOG) performance status (PS) of 0 to 1.

8. Adequate organ function and bone marrow reserve as indicated by the following laboratory assessments performed within 14 days prior to the leukapheresis for manufacture of autologous blood product:
a. Bone marrow function: absolute neutrophil count ≥1000/μL; hemoglobin ≥9 g/dL; platelet count ≥75,000/μL. Note: Blood transfusion within 4 weeks prior to blood draw for autologous blood product manufacture is not permitted; however, patients can receive blood transfusions following the blood draw as clinically indicated.
b. Hepatic function: total serum bilirubin≤1.5×upper limit of normal (ULN); serum aspartate aminotransferase (AST)/alanine aminotransferase (ALT)≤2.5×ULN (≤5×ULN in the presence of hepatic metastasis), and alkaline phosphatase <2.5×ULN with the following exception:
   Patients with liver and bone involvement: alkaline phosphatase≤5×ULN
   Patients with inherited disorders of bilirubin metabolism (discussed with Sponsor)
c. Renal function: serum creatinine≤2.5×ULN or creatinine clearance ≥50 mL/min based on either urine collection or Cockcroft-Gault estimation.
d. Coagulation profile: prothrombin time (PT), international normalized ratio (INR) and partial thromboplastin time (PTT)≤1.5×ULN. Patients on a stable, maintenance regimen of anticoagulant therapy for at least 30 days prior to leukapheresis for manufacture of autologous blood product can have PT/INR measurements >1.5×ULN if, in the opinion of the Investigator, the patient is suitable for the study. An adequate rationale must be provided to the Sponsor prior to enrollment.

9. Female patients of childbearing potential must: a) have a negative serum beta human chorionic gonadotropin (β-hCG) pregnancy test at Screening; and b) agree to use highly effective contraception from the time of informed consent until at least 5 months after the last dose of pembrolizumab or drug product (Clinical Trials Facilitation and Coordination Group, CTFG, 2020).

10. Male patients who are not vasectomized must be willing to use condoms from the time of informed consent until at least 5 months after the last dose of drug product or pembrolizumab.

### Patients in Part 1A and Part 1B

11. For cervical cancer, which is not amenable to curative treatment with surgery, radiation, and/or chemoradiation therapy, patients must have progressive disease after receiving prior treatments, which include at least 1 prior systemic chemotherapeutic treatment with a platinum-based regimen in the adjuvant or recurrent setting and ICI (received or been offered). Patients who relapsed after platinum-containing definitive chemoradiation or after adjuvant chemoradiation are eligible if a platinum re-challenge at time of relapse is not seen as beneficial. For safety intolerant to a platinum-based systemic chemotherapeutic treatment for recurrent disease, reasons must be documented.

12. For head and neck cancer, which is not amenable to curative treatment with surgery, radiation, and/or chemoradiation therapy, the cancer must have progressed following at least 1 prior platinum-based chemotherapy in the primary, adjuvant or recurrent setting and have received or been offered checkpoint immunotherapy. Patients who relapsed after platinum-containing definitive chemoradiation or after adjuvant chemoradiation are eligible if a platinum re-challenge at time of relapse is not seen as beneficial. For patients intolerant to platinum-based chemotherapy for recurrent disease, reasons must be documented.

13. For incurable or metastatic cancers other than cervical or head and neck cancer patients must have been offered all available standard of care (SOC) or progressed from all available standard therapies, or the patient is intolerant to standard therapy (ies) or has a tumor for which no standard therapy (ies) exists according to National Comprehensive Cancer Network (NCCN) Clinical Practice Guidelines (www.nccn.org/guidelines/guidelines-process/development-and-update-of-guidelines).

14. Patients with immune-mediated endocrinopathies following treatment with immune checkpoint inhibitors requiring hormone replacement therapy are eligible. Note:

Patients requiring prednisone as part of hormone replacement therapy are eligible if the daily dose does not exceed 10 mg.

### Patients in Part 2

15. Immune checkpoint inhibitor treatment-naive patients. Patients can have received other anti-cancer therapies for the treatment of the disease under study.

### Exclusion Criteria

1 Treatment with anticancer therapy, including investigational therapy, within 2 weeks prior to leukapheresis for manufacture of autologous blood product. For prior therapies with a half-life longer than 3 days, the interval must be at least 28 days prior to the first administration of study treatment.

2. With Grade >1 AEs (except Grade 2 neuropathy, ototoxicity, mucositis, fatigue, alopecia, and endocrine disorders managed with hormone replacement) according to National Cancer Institute (NCI) Common Terminology Criteria for Adverse Events (CTCAE) version 5.0 related to previous treatment with anticancer or investigational therapy that do not resolve (i.e., Grade <2) at least 2 weeks prior to leukapheresis for manufacture of autologous blood product.

3. With Grade ≥3 AEs related to prior surgery, radiation, or disease that do not resolve to Grade ≤2 as well as patients with any opportunistic or active systemic infection that does not resolve prior to enrollment.

4. Treated with non-corticosteroid based immunosuppressive agents within the last 6 months prior to leukapheresis.

5. With active, known, or suspected autoimmune disease, with the exception of autoimmune-related hypothyroidism on thyroid-replacement therapy, patients with controlled Type 1 diabetes mellitus, patients with eczema, psoriasis, lichen simplex or vitiligo with dermatologic manifestations only.

6. Patients with prior allogeneic bone marrow or solid organ transplantation.

7. Live virus vaccination within 4 weeks prior to leukapheresis for manufacture of autologous blood product.

8. Systemic treatment with either corticosteroids (>10 mg of prednisone or the equivalent per day) or other immunosuppressive medications within 14 days prior to leukapheresis for manufacture of autologous blood product. Inhaled, intranasal, intra-articular and topical (including ocular) steroids are allowed. The use of steroid replacement for patients with adrenal insufficiency is allowed. The use of fludrocortisone for mineralocorticoid replacement in patients with adrenal insufficiency is allowed.

9. Known active central nervous system metastases and/or carcinomatous meningitis. Patients with previously treated brain metastases can participate provided they are stable (without evidence of progression by imaging for at least 4 weeks prior to the first dose of investigational product and any neurologic symptoms have returned to Baseline), have no evidence of new or enlarging brain metastases, and are not using steroids for at least 7 days prior to leukapheresis for manufacture of autologous blood product. This exception does not include carcinomatous meningitis, which is excluded regardless of clinical status.

10. Patients with active interstitial lung disease and any history of myocarditis.

11. Clinically significant cardiac disease, including unstable angina, acute myocardial infarction within 6 months prior to leukapheresis for manufacture of autologous blood product, New York Heart Association class III or IV congestive heart failure, and arrhythmia requiring therapy.

12. Systemic arterial thrombotic or embolic events, such as cerebrovascular accident (including ischemic attacks) within 1 month prior to leukapheresis for manufacture of autologous blood product.

13. Systemic venous thrombotic events (eg, deep vein thrombosis) or pulmonary arterial events (eg, pulmonary embolism) within 1 month prior to leukapheresis for manufacture of autologous blood product. Patients with venous thrombotic events before leukapheresis for manufacture of autologous blood product on stable anticoagulation therapy are eligible.

14. History or presence of an abnormal electrocardiogram (ECG) that is clinically meaningful.

15. Left ventricular ejection fraction (LVEF)<50%.

16. Major surgery within 2 weeks of leukapheresis for manufacture of autologous blood product. Following major surgeries >2 weeks prior to leukapheresis for manufacture of autologous blood product, all surgical wounds must be healed and free of infection or dehiscence.

17. Any other clinically significant comorbidities, such as active infection, known psychiatric or neurological disorder, or any other condition, which in the judgment of the Investigator, could compromise compliance with the protocol, interfere with the interpretation of study results, or predispose the patient to safety risks.

18. Known human immunodeficiency virus (HIV) infection, or active hepatitis B, hepatitis C, or Mycobacterium tuberculosis infection.

19. Female patients who are breastfeeding or have a positive serum pregnancy test at the Screening visit.

20. History of allergy or hypersensitivity to any component of drug product. History of alcohol and/or illicit drug abuse within 12 months of signing the ICF.

### Patients in Part 1B.

22. History of severe allergic anaphylactic reactions to chimeric, human, or humanized antibodies or infusion proteins.

23. Known hypersensitivity to Pembrolizumab, Chinese hamster ovary cell products, or any component of the Pembrolizumab formulation.

24. History of any Grade 3 immune-related AE (irAE) from prior immunotherapy (patients with endocrinopathy managed with replacement therapy or asymptomatic elevation of serum amylase or lipase are eligible), any irAE that led to permanent discontinuation of prior ICI.

### Patients in Part 2

25. Prior treatment with ICI (including but not limited to anti-PD-1, anti-PD-L1, anti-CTLA4, or anti-LAG3, etc.). Other investigational immunotherapy agents must be discussed.

26. History of severe allergic anaphylactic reactions to chimeric, human, or humanized antibodies or infusion proteins.

### Leukapheresis

The goal of the leukapheresis is to provide a yield of PBMCs for each patient of approximately 10 to 14×10⁹ cells to support full treatment duration. Efforts should be made to adjust the procedure in case a low yield is expected. In accordance with local procedures, a white blood cell (WBC) or complete blood cell (CBC) count should be taken during leukapheresis so that the processed blood volume can be increased. In the event a WBC or CBC count cannot be done during leukapheresis, a sample should be taken at the end of leukapheresis to determine the WBC count in the leukopak. The results should be processed as soon as possible and provided to the Sponsor in real-time. Concomitant medications should be collected.

### Tumor Response Assessment and Schedule

Tumor assessment will be performed by the Investigative site at Screening (Baseline), and (+7 days) every 6 weeks for the first 2 tumor assessments, then every 8 weeks until the end of first year, thereafter every 12 weeks for the second year, or until disease progression as confirmed by RECIST 1.1 and iRECIST, subsequent treatment initiated, unacceptable toxicity, withdrawal of consent, or death, whichever occurs first. This is to be done via imaging. Patients who experience disease progression per RECIST 1.1 can continue dosing if considered in their best interest by the treating Investigator to allow for confirmation of disease progression, ie, iCPD according to iRECIST.

Tumors biopsied for fresh tissue collection during screening should not be followed for radiographic response.

If a patient discontinues the investigational product for reasons other than disease progression, the patient should continue to be imaged following the schedule outlined above. If a patient discontinues treatment due to clinical deterioration, the AEs associated with the clinical progression should be recorded on the AE page. Radiographic assessments should be obtained and recorded in the eCRF according to Schedule of Assessments and Procedures.

At screening and all subsequent time points, cervical, anal/rectal, vulvar/vaginal, and penile carcinomas require computed tomography (CT) of the torso (chest, abdomen, and pelvis) and all known sites of disease; oropharyngeal carcinomas require CT of head, neck, chest, and other areas of known involvement. If CT scans cannot be used or do not allow for an appropriate tumor assessment, magnetic resonance imaging (MRI) is permitted. However, the radiographic procedure used for tumor assessment at screening should be used throughout the study. For all other advanced solid tumor types, the Investigator should image all known sites of disease using the imaging modality the Investigator believes best for that tumor type.

Brain MRI is required at screening of all patients with a history of brain metastases and can be repeated at subsequent time points in any patient with a history of brain metastases and/or in any patient who develops symptoms indicative of brain metastasis. If a patient is unable to tolerate or has a contraindication for MRI, CT scan can be used.

The same evaluator should perform assessments to ensure internal consistency across visits. At the Investigator's discretion, CT scans should be repeated at any time if PD is suspected. For patients who achieve a partial response (PR) or complete response (CR), tumor assessment should be repeated 4 weeks later to confirm response.

### Pharmacodynamic Assessments, Including Immunogenic Measurements Sample Collection Schedule

### Tumor Biopsies

Prior to treatment initiation (Cycle 1, Day 1), patients will undergo a tumor biopsy (primary tumor or metastasis), which can also be obtained from a previously radiated site that has active tumor growth. Tumors biopsied for fresh tissue collection during screening should not be followed for radiographic response. All patients are required to undergo an additional tumor biopsy of the same primary tumor or metastasis on Cycle 2 Day 8 (+2 days).

An optional tumor biopsy is requested (pre-dose) and/or if the patient progresses or discontinues treatment. If possible, another mass from the same anatomical location should be obtained if biopsy of the same site as biopsied at screening cannot be performed. These unscheduled biopsies can also be obtained at additional timepoints to further characterize response mechanisms if the patient has consented.

Tumor tissue should be of good quality based on total and viable tumor content. Archived tumor tissue can be provided for central confirmation of HPV-16 positivity, if required or applicable.

### Pharmacodynamic Assessments

Baseline and post-treatment blood and tumor samples will be collected for pharmacodynamic and biomarker assessments according to the Schedule of Assessments and Procedures. Tests include, but are not limited to, immunophenotyping, measurements of T cell biomarkers and cytokine production, endogenous immune responses, and HPV-16 cfDNA. Changes in circulating blood cells and cellular responses in tumor biopsies can also be evaluated. Tumor tissue will be analyzed via immunohistochemistry and/or other related techniques to assess biomarkers related to cancer, inflammation, immune responses, changes in tumor micro-environment, and related areas of interest. Additional pharmacodynamic assessments and biomarkers can be investigated as they are identified throughout the course of the clinical study.

### Optional Pharmacogenomic Analysis

Optional pharmacogenomic assessments on blood sample(s) and/or tumor tissue samples(s) can be performed. These optional tests can include but are not limited to transcriptome profiling and T cell receptor sequencing. No additional samples are required for this testing to be completed. The testing will be performed if the patient has provided a consent form.

### Cytokine Assessments

Blood samples will be collected from all patients for cytokines as per the Schedule of Assessments and Procedures. Patients with Grade 2, 3, or 4 CRS will have additional cytokine plasma levels performed during Grade 2, 3, or 4 CRS events. Blood collections should be obtained at the time of diagnosis of a CRS, at the time of an increase in severity (e.g., when a Grade 2 CRS progresses to a Grade 3 CRS), at the onset of neurological symptoms, and at the time of discharge or resolution.

The evaluation of a cytokine panel will include, but is not limited to, IFN gamma (IFNγ) and IL-6. Although CRS can have a delayed onset, it rarely presents beyond 14 days after initiation of therapy. Patients exhibiting symptoms consistent with CRS presenting outside this window should be carefully evaluated for other causes.

Cytokines will also be monitored for pharmacodynamic assessments. Baseline and post-treatment serum samples will be collected to assess anti-tumor immune responses by measuring cytokines that could provide information about drug inflammatory responses.

### Safety Assessments

Safety is evaluated in this study through the monitoring of all SAEs and nonserious AEs and laboratory abnormalities, defined and graded according to NCI CTCAE version 5.0. General safety assessments include physical examinations and specific laboratory evaluations, including serum chemistry, coagulation, and blood counts including differential. SAEs and >Grade 2 AESIs will be reported in an expedited fashion for entry into the safety database.

During the conduct of the study, the totality of safety events observed is reviewed (including CRS events that resolved to Grade 2) and a decision is made if a given event requires initiation of staggered enrollment of patients following this event. Staggered enrollment in potential additional monotherapy cohorts (Part 1) or in the Combination Safety Cohorts (Part 2) require all subsequent newly enrolled patients in a cohort or cohorts to be staggered by 1 week. Semi sequential enrollment of patients can continue in some cohorts, if applicable. Patients will have cytokine release assays performed during the study. Patients with Grade 2, 3, or 4 CRS have additional blood samples taken for safety laboratories and the evaluation of the cytokine panel.

Exposure to immune checkpoint inhibitors can increase the risk of irAEs, specifically autoimmune conditions. As such, irAEs are recognized early and treated promptly to avoid potential major complications.

All patients return to the clinic for a Safety Follow-up visit within 15 to 45 days after the last dose of investigational product. All AEs and SAEs are recorded until 6 weeks after last dose of investigational product (EOD6W) or up to 45 days from drop out or until initiation of another anticancer therapy, whichever occurs first. Only ongoing SAEs determined by the Investigator to be possibly, probably, or definitely related to Drug product monotherapy or combination therapy are followed up.

### Physical Examination and Height and Weight

A physical examination includes height (screening only), weight, and an assessment of general appearance and an evaluation of the following systems: dermatologic, head, eyes, ears, nose, mouth/throat/neck, thyroid, lymph nodes, respiratory, cardiovascular, gastrointestinal, extremities, musculoskeletal, neurologic, and gynecologic and genitourinary systems, as indicated. It is especially important to capture weight during the physical examination of the patient within 24 hours of leukapheresis, as patient dosing is determined by weight. Vital signs are collected and include measurement of systolic and diastolic blood pressure while the patient is in a seated position, heart rate, temperature, and respiratory rate.

### Performance Status

Eastern Cooperative Oncology Group scales and criteria are used to assess a patient's performance status, assess how the disease affects the daily living abilities of the patient, and determine appropriate treatment and prognosis.

### Vital Signs

Vital signs are collected and include measurement of systolic and diastolic blood pressure while the patient is in a seated position, heart rate, temperature, and respiratory rate.

### 12-Lead Electrocardiograms

12-lead ECGs are performed by qualified site personnel using an ECG machine that determines heart rate, PR interval, QRS interval, RR interval, QT interval, and QTc interval collected by QTcB (QTc corrected by Bazett's formula) and QTcF (QTc corrected by Fridericia's formula). During the collection of ECGs, patients are in a resting position, in a quiet setting without distractions (e.g., television, cell phones) for at least 10 minutes before ECG collection. All ECGs must be evaluated by a qualified physician for the presence of abnormalities.

Echocardiogram or multigated acquisition (MUGA) scans are performed to measure LVEF at Screening and as clinically indicated.

### Laboratory Assessments

Samples for clinical laboratory assessments will be collected. Clinical laboratory tests outlined in Table 3 are performed by the site. Samples for laboratory tests outlined in Table 3 are collected in appropriate tubes and handled according to standard procedures of the site. Clinical laboratory variables are listed in Table 3.

TABLE 3
Clinical Laboratory Assessments
Hematology ^{a}

| | |
|---|---|
| Total white blood cell count | Neutrophils (absolute count) |
| Red blood cell count | Lymphocytes (absolute count) |
| Hemoglobin | Monocytes (absolute count) |
| Hematocrit | Eosinophils (absolute count) |
| Mean corpuscular volume | Basophils (absolute count) |
| Mean corpuscular hemoglobin | Platelet count |
| Mean corpuscular hemoglobin | Red blood cell distribution width |
| concentration | |

Coagulation ^{a, b}

| | |
|---|---|
| Prothrombin time | International normalized ratio |
| Partial thromboplastin time | |

Coagulation (in Cases of CRS only)

| | |
|---|---|
| Fibrinogen | D-dimer |
| von Willebrand factor | |

Serum Chemistry

| | |
|---|---|
| Alanine aminotransferase | Gamma glutamyl transferase |
| Albumin | Glucose |
| Alkaline phosphatase | Lactate dehydrogenase |
| Aspartate aminotransferase | Phosphorus |
| Blood urea nitrogen | Potassium |
| Calcium | Sodium |
| Chloride | Thyroid function test |
| | (TSH, free T3, and free T4) |
| Creatinine | Total bilirubin |
| C-reactive protein | Total protein |
| Creatine kinase | Uric acid |
| Ferritin | Magnesium |

Urinalysis

| | |
|---|---|
| Bilirubin | Blood |
| Glucose | pH and specific gravity |
| Ketones | Protein |
| Leukocytes | Urobilinogen |
| Nitrite | Leukocyte esterase |

Microscopic (if macroscopic panel is abnormal) white blood cells,
red blood cells, casts, crystals, bacteria, and epithelial cells
Viral Serology

| | |
|---|---|
| Hepatitis B core antibody | Human immunodeficiency virus |
| IgM antibody to anti-HBC | (Types 1 and 2) antibodies |
| Hepatitis B surface antigen | Hepatitis C virus antibody |

Pregnancy Testing
Serum β-hCG (women of childbearing potential only) required
at screening; urine hCG can be used in subsequent assessments.
Abbreviations: β-hCG = beta-human chorionic gonadotropin; CRS = cytokine-release syndrome; T3 = triiodothyronine; T4 = thyroxine; TSH = thyroid-stimulating hormone.
^{a} These laboratory tests are required to be collected prior to or on the day of leukapheresis, with the results available for review prior to leukapheresis.
^{b} Results of coagulation parameters should be available for review (by Investigator or designee) on the day of or the day before any tumor biopsy.

### Adverse Events

Samples for clinical An AE is any untoward medical occurrence in a patient that does not necessarily have a causal relationship with the investigational product administered. An AE can therefore be any unfavorable or unintended sign (including an abnormal laboratory finding), symptom, or disease temporally associated with the use of an investigational product, whether or not related to the investigational product. Adverse events can be new events or can be pre-existing conditions that have become aggravated or have worsened in severity or frequency. Adverse events can be clinically significant changes from Baseline in physical examination, laboratory tests, or other diagnostic investigation. In this study, an AE is treatment-emergent if the onset time is after administration of investigational product through 6 weeks after the last dose of investigational product.

### Serious Adverse Event (SAE)

An SAE is any AE that results in any of the following:

### Death

Is immediately life-threatening
Requires in-patient hospitalization or prolongation of existing hospitalization
Results in persistent or significant disability or incapacity
Results in a congenital abnormality or birth defect
Is an important medical event that can jeopardize the patient or can require medical intervention to prevent 1 of the outcomes listed above

All SAEs that occur after any patient has signed the ICF, before treatment, during treatment, or within 30 days following the cessation of treatment, whether or not they are related to the study, must be recorded on the appropriate clinical procedure form.

### Adverse Events of Special Interest

An AESI is an AE (serious or nonserious) of scientific and medical concern specific to investigational product, for which ongoing monitoring and immediate notification by the Investigator to the Sponsor is required. Such AEs can require further investigation to characterize and understand them. Adverse events of special interest can be added or removed during the study by a protocol amendment. The following AEs are considered AESIs:
Events suggestive of hypersensitivity, cytokine release, systemic inflammatory response syndrome, systemic inflammatory activation
Influenza-like illness
Infusion-reaction syndrome
irAEs related to immune therapy, such as myocarditis, neurological irAEs, transaminitis of immune-related etiology, and nephritis

In addition, the following events are reported to the Sponsor:
Liver tests abnormalities meeting Hy's law criteria, i.e., an AST or ALT laboratory value >3×ULN and a total bilirubin laboratory value >2×ULN and, at the same time, an alkaline phosphatase laboratory value <2×ULN, as determined by protocol specified or unscheduled laboratory testing.

### Assessment of Severity

The NCI CTCAE version 5.0 is used to assess and grade severity for AEs and for laboratory abnormalities. ASTCT Consensus Grading will be used for CRS and ICANS. Each AE term will be mapped to the latest version of Medical Dictionary for Regulatory Activities (MedDRA) term and code. If the event is not covered in CTCAE version 5.0, the guidelines shown in Table 4 should be used to assess severity.

TABLE 4
Severity and Toxicity Grade of Events Not Covered by CTCAE

| Toxicity | | |
|---|---|---|
| Grade | Severity | Description |
| Grade 1 | Mild | Transient or mild discomfort (<48 hours); |
| | | no medical intervention/therapy required. |
| Grade 2 | Moderate | Mild to moderate limitation in activity - |
| | | some assistance can be needed; no or minimal |
| | | medical intervention/therapy required. |
| Grade 3 | Severe | Marked limitation in activity, some |
| | | assistance usually required; medical |
| | | intervention/therapy required, |
| | | hospitalization possible. |
| Grade 4 | Life- | Extreme limitation in activity, significant |
| | threatening | assistance required; significant medical |
| | | intervention/therapy required, |
| | | hospitalization or hospice care possible. |
| Grade 5 | Fatal | The patient died due to the event. |

Source: (NIAID, 2003)
Abbreviations: CTCAE = Common Terminology Criteria for Adverse Events

### Assessment of Causality

The Investigator(s) will provide causality assessment and relationship of AEs and SAEs to the study treatment. For patients receiving combination therapy (Part 1B and Part 2), causality will be assessed individually for each protocol-specified therapy. A reasonable suspected causal relationship should be attributed to the Pembrolizumab alone if the event is consistent with the Pembrolizumab prescribing labeling.

TABLE 5
Investigator Assessed Causality Relationship of Adverse Events

| Relationship | Description |
|---|---|
| Not Related | The event is clearly related to factors other than study treatment, such as the patient's clinical |
| | state, therapeutic interventions, or concomitant medications administered to the patient. |
| Unlikely | The temporal association, patient history and/or circumstances are such that the study drug or |
| Related | treatment is not likely to have had an association with observed event. |
| Possibly | The event follows a reasonable temporal sequence from the time of study treatment |
| Related | administration, and/or follows a known response pattern to study treatment, but could have |
| | been produced by other factors, such as the patient's clinical state, therapeutic interventions, |
| | or concomitant medications administered to the patient. |
| Probably | The event follows a reasonable temporal sequence from the time of study treatment |
| Related | administration, and follows a known response pattern to study treatment, and cannot be |
| | reasonably explained by other factors, such as the patient's clinical state, therapeutic |
| | interventions, or concomitant medications administered to the patient. |
| Definitely | The event follows a reasonable temporal sequence from the time of study treatment |
| Related | administration, and follows a known response pattern to study treatment, and cannot be |
| | reasonably explained by other factors, such as the patient's clinical state, therapeutic |
| | interventions, or concomitant medications administered to the patient. |
| | In addition, the event either occurs immediately following study treatment |
| | administration, improves on stopping study treatment, reappears on repeat exposure, or |
| | there is a positive reaction at the application site. |

### Expectedness

An AE that is not listed in, or is inconsistent with the specificity or severity, from the applicable product information (e.g., the IB for Drug product or the approved labeling for atezolizumab, ipilimumab, or nivolumab) is considered unexpected.

### Efficacy Analyses

Progression-free Survival (PFS) is defined as the time from Cycle 1 Day 1 to first documentation of objective tumor progression (PD, radiological) according to RECIST 1.1 or death due to any cause, whichever comes first. Progression-free survival data will be censored on the date of last tumor assessment documenting absence of PD for patients who do not have objective tumor progression and are still on study at the time of the analysis, are given antitumor treatment other than investigational product, or are removed from treatment follow-up prior to documentation of objective tumor progression. Patients having no tumor assessments after enrollment who are not known to have died will have PFS censored on Cycle 1 Day 1. PFS will be assessed by both RECIST 1.1 and iRECIST criteria to accommodate different practice across participating sites.

Overall Survival (OS) is defined as the time from the date of Cycle 1 Day 1 to date of death due to any cause. In the absence of confirmation of death, survival time will be censored at the last date the patient is known to be alive. Patients lacking data beyond Cycle 1 Day 1 will have their survival times censored on Cycle 1 Day 1.

Objective Response Rate (ORR) is defined as the proportion of patients with CR or PR according to RECIST 1.1. Objective response rate will be provided as unconfirmed and confirmed ORR. Confirmed responses are those that persist on repeat imaging study at least 28 days after the initial documentation of response. Similarly, iORR by iRECIST will also be summarized and reported.

Time to Response is defined as the time (in months) from the start of the study treatment to first incidence of CR or PR by RECIST 1.1. This will be calculated only for patients who achieve CR or PR.

Duration of Response (DoR) is defined as the time from the first documentation of PR or CR to the first documentation of objective tumor progression or death due to any cause. Duration of response data will be censored on the day of the last tumor assessment documenting absence of PD for patients who do not have tumor progression and are still on the study at the time of an analysis, are given antitumor treatment other than the investigational product, or are removed from the study follow-up prior to documentation of objective tumor progression will be censored at the last tumor assessment. Similarly, iDoR by iRECIST will also be summarized and reported.

Best Overall Response (BOR) is determined once all tumor assessments from Cycle 1 Day 1 until disease progression or death are recorded. In general, it is the best response across all assessments; however, confirmation of CR, PR, and stable disease (SD) will also be used in BOR determination. To confirm CR or PR, changes in tumor measurements must be confirmed by repeat assessments that should be no less than 4 weeks (28 days) after the criteria for response are first met. To confirm SD, it must have occurred at least 12 weeks from Cycle 1 Day 1; otherwise, BOR will depend on subsequent assessments. Best overall response will be summarized by percentages and as a time to event variable for time to best response using enrollment as the anchor date. Similarly, iBOR by iRECIST will also be summarized and reported.

Disease Control Rate (DCR) is the proportion of patients in whom the BOR is determined as CR, PR, or SD by RECIST 1.1 at defined time points. All patients in the safety population with measurable disease at Baseline and eligible for tumor assessment will be considered as the denominator of the DCR proportion at 3, 6, and 12 months. Similarly, iDCR by iRECIST will also be summarized and reported.

Stable Disease 12 weeks is the proportion of patients for whom the BOR is determined as CR, PR, or SD by RECIST 1.1 and is maintained for at least 12 week. All patients in the safety population with measurable disease at Baseline and eligible for tumor assessment will be considered as the denominator of the proportion at 3, 6, and 12 months. Similarly, SD for 12 weeks by iRECIST will also be summarized and reported.

The efficacy analyses will be performed on the safety population. Best overall response will be reported as incidence and percentage. Efficacy parameters, OS, PFS, Time to Response, and DOR will be described using Kaplan-Meier analyses. Proportions such as ORR, DCR, and patients with SD ≥12 weeks will be reported. When meaningful sample sizes are available, 95% confidence intervals will also be reported. If the Efficacy-evaluable population differs from the Safety population, efficacy analyses will also be performed using the Efficacy-evaluable population. All assessments using response assessments by RECIST 1.1 or iRECIST will be analyzed using the Investigators' review assessments.

The Kaplan-Meier method is used to estimate the median PFS and 2-sided 95% confidence interval. Patients who die, regardless of cause of death, are considered to have had an event unless subsequent anticancer therapy was received prior to death. If subsequent therapy is received, the patient will be censored of date of last evaluable tumor assessment prior to subsequent therapy. Patients who withdraw consent for the study are considered censored at the time of the last evaluable tumor assessment prior to withdrawing consent. Patients who are still alive at the time of the clinical data cut-off date are censored at the most recent evaluable tumor assessment. All patients who were lost to follow-up prior to the clinical data cut-off date will also be considered censored at the time of the last evaluable tumor assessment prior to lost to follow up.

Duration of response, time to best overall response, and overall survival will use the same method as PFS. In addition, iPFS, iBOR, iDCR, and time to iBOR using iRECIST are analyzed and reported using similar methods.

Objective Response Rate (ORR) are presented as a proportion with a 95% confidence interval based on the exact binomial distribution. The point estimate and 2-sided 95% confidence interval of ORR will be provided. DCR and SD lasting at least 12 weeks will be reported as point estimates.

### Safety Analyses

All safety parameters are analyzed using the Safety population. Safety parameters include: AEs, laboratory evaluations, vital signs, ECOG, exposure, ECG, ECHO/MUGA and physical examinations.

The primary endpoint for safety is the number of patients with any AE and observed toxicity to Drug product administration, where the severity is assessed using NCI CTCAE version 5.0. All AEs with onset after the first administration of Drug product are included in the analysis. Adverse events are collected beginning at signing informed consent; however analyses is performed focusing on treatment-emergent AEs.

AEs are analyzed using descriptive statistics. For patients with multiple incidences of a given AE, the highest severity is used.

### Adverse Events

Adverse events will be collected from the time each patient signs the initial ICF through EODW6. The AEs will be coded using the MedDRA dictionary. Adverse events considered as possibly, probably, or definitely related to investigational product by the Investigator will be classified as related for summary purposes. All AEs are provided in patient listings.

### Clinical Laboratory Evaluation

The baseline is defined as the last non-missing value prior to the first exposure to investigational product. This is typically Cycle Day 1 pre-dose, but can be earlier. Actual values and changes from Baseline clinical laboratory tests are summarized by study visit.

Laboratory test results are classified according to NCI CTCAE version 5.0 and clinical significance as determined by the Investigator. If more than 1 laboratory result is reported per study visit per parameter, the result yielding the most severe classification is selected for analysis. Shift tables are created to show the greatest change from baseline for graded laboratory parameters. All laboratory assessments are provided in listings.

Patients with clinically significant abnormal laboratory test results are listed. This listing includes all results of the laboratory parameter that was abnormal and determined to be clinically significant by the Investigator for a patient across study visit.

### Vital Signs

Vital sign values are classified according to the clinical significance, as determined by the Investigator. The number of patients with a non-missing result, the number and percentage of patients with a non-clinically significant result, and clinically significant result is summarized by study visit and study time point. If more than one vital sign result is reported per study visit and study time point per parameter, the result yielding the most severe classification is selected for analysis.

Patients with clinically significant vital sign values are listed. This listing includes all results of the vital sign parameter that was determined by the Investigator to be clinically significant for a patient across study time points.

### Physical Examination

Abnormal physical examination findings are listed.

The ECG results are presented in a shift table (normal, abnormal not clinically significant, abnormal, clinically significant) to show the greatest change from baseline. All ECG results are presented in patient listings.

All safety data is provided in listings.

The ECOG PS and change from Baseline in ECOG PS are summarized at each scheduled visit that it is collected. Change from Baseline in ECOG PS is summarized as a continuous variable and as a categorical variable. A decrease of ≥1 point from Baseline is categorized as an “improvement” from Baseline. An increase of ≥1 point from Baseline is categorized as a “deterioration” from Baseline. Improvement, deterioration, and unchanged ECOG PS from Baseline is summarized as a categorical variable by treatment at each post-enrollment time point that ECOG PS is evaluated.

### Pharmacodynamic Analyses

The biomarkers analyses in this study are exploratory, and will be summarized for each time point, for change from Baseline and percentage change from Baseline. Correlation between pharmacodynamic markers and Drug product dose can be explored with descriptive statistics and graphical methods. Descriptive statistics (mean, standard deviation, median, minimum, maximum, and geometric mean, if applicable, for each biomarker will be reported. Graphs of individual values over time according to dose group will be presented.

### Dose Manufacture Feasibility

Dose manufacturing feasibility will be assessed based on individual patient batch yield, product failures prohibiting use, and any additional information from leukapheresis through Drug product product production that is deemed relevant.

### Example 2. Preliminary Efficacy

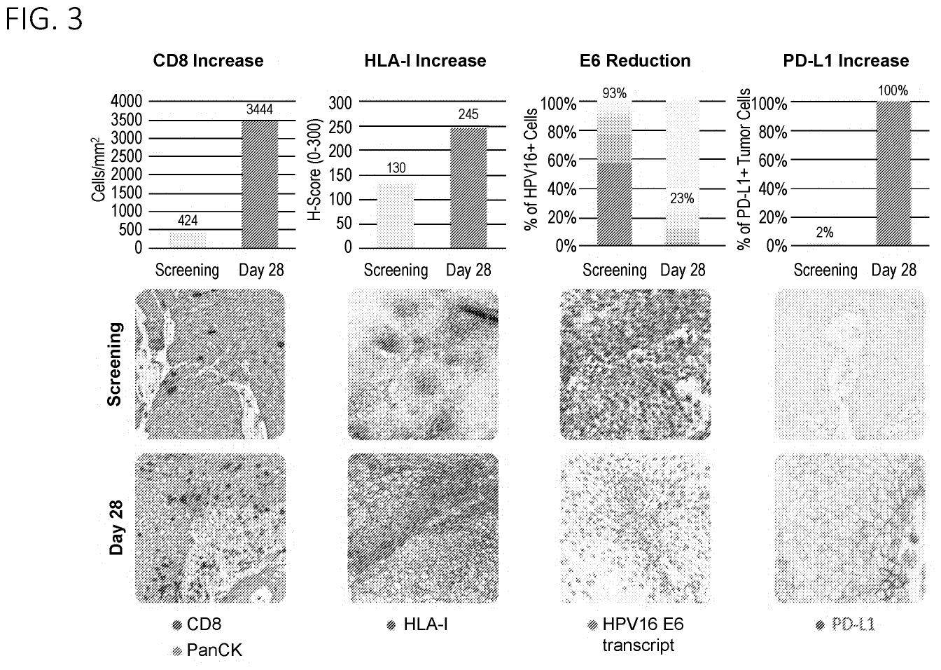

Two patients from the lower dose cohorts have demonstrated a substantial increase in their CD8+ T cell tumor infiltration and had stable disease. One of these 2 patients who had a lower tumor burden has remained on the study for more than 10 months and appears to be deriving clinical benefit. Of the 5 evaluable patients from the highest dose cohort, which has been established as the RP2D, 1 patient with checkpoint refractory head-and-neck cancer had a clinical response and symptomatic improvement. The target lesion demonstrated a complete response at both radiographic assessments. At the most recent assessment, the major oropharyngeal lesion demonstrated continued improvement upon physical examination; however, a new dermal lesion was detected. This patient showed the highest increase in tumor-infiltrating CD8+ T-cells with concomitant reduction in cells expressing E6 and E7 (not shown). Increased PD-L1 expression also suggests potential synergy with combination therapy, as shown in FIG. 3.

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, can need to be revisited. Further, the Examiner is also reminded that any disclaimer made in the instant application should not be read into or against the parent application.

TABLE B
Exemplary Sequences:

| | | |
|---|---|---|
| 1 | ATGATGGTGGACGGCGACAACAGCCACGTGGAAATGAAGCTG | TFRC_G4S_IFN- |
| | GCCGTGGACGAGGAAGAGAACGCCGACAACAACACCAAGGCC | a2a mRNA |
| | AACGTGACCAAGCCTAAGAGATGCAGCGGCAGCATCTGCTAC | |
| | GGCACAATCGCCGTGATCGTGTTCTTCCTGATCGGCTTTATG | |
| | ATCGGCTACCTGGGCTACTGCAAGAGCAGTGATGGACCTGGC | |
| | GAAACAGGCGGAGGCGGAGGATCTGGTGGCGGAGGAAGCGGT | |
| | GGCGGCGGATCTTGTGATCTGCCTCAGACACACAGCCTGGGC | |
| | AGCAGACGAACACTGATGCTGCTGGCCCAGATGCGGAAGATC | |
| | AGCCTGTTCAGCTGCCTGAAGGACCGGCACGATTTCGGCTTC | |
| | CCTCAAGAGGAATTCGGCAACCAGTTCCAGAAGGCCGAGACA | |
| | ATCCCTGTGCTGCACGAGATGATCCAGCAGATCTTCAACCTG | |
| | TTCTCCACCAAGGACAGCAGCGCCGCCTGGGATGAGACACTG | |
| | CTGGACAAGTTCTACACCGAGCTGTACCAGCAGCTGAATGAC | |
| | CTGGAAGCCTGCGTGATCCAAGGCGTGGGAGTGACAGAGACA | |
| | CCCCTGATGAAGGAAGATAGCATCCTGGCCGTGCGCAAGTAC | |
| | TTCCAGCGGATCACCCTGTACCTGAAAGAGAAGAAGTACAGC | |
| | CCCTGCGCCTGGGAAGTCGTGCGGGCCGAAATCATGAGAAGC | |
| | TTCAGCCTGAGCACCAACCTGCAAGAGAGCCTGCGGAGCAAA | |
| | GAGTGA | |
| 2 | ATGATGGTGGACGGCGACAACAGCCACGTGGAAATGAAGCTG | TFRC_G4S_IL-12 |
| | GCCGTGGACGAGGAAGAGAACGCCGACAACAACACCAAGGCC | mRNA |
| | AACGTGACCAAGCCTAAGAGATGCAGCGGCAGCATCTGCTAC | |
| | GGCACAATCGCCGTGATCGTGTTCTTCCTGATCGGCTTTATG | |
| | ATCGGCTACCTGGGCTACTGCAAGAGCAGTGATGGACCTGGC | |
| | GAAACAGGCGGAGGCGGAGGATCTGGTGGCGGAGGAAGTGGC | |
| | GGCGGAGGTTCTATTTGGGAGCTGAAGAAAGACGTGTACGTG | |
| | GTGGAACTGGACTGGTATCCCGATGCTCCTGGCGAGATGGTG | |
| | GTGCTGACCTGCGATACCCCTGAAGAGGACGGCATCACCTGG | |
| | ACACTGGATCAGTCTAGCGAGGTGCTCGGCAGCGGCAAGACC | |
| | CTGACCATCCAAGTGAAAGAGTTTGGCGACGCCGGCCAGTAC | |
| | ACCTGTCACAAAGGCGGAGAAGTGCTGAGCCACAGCCTGCTG | |
| | CTGCTCCACAAGAAAGAGGATGGCATTTGGAGCACCGACATC | |
| | CTGAAGGACCAGAAAGAGCCCAAGAACAAGACCTTCCTGAGA | |
| | TGCGAGGCCAAGAACTACAGCGGCCGGTTCACATGTTGGTGG | |
| | CTGACCACCATCAGCACCGACCTGACCTTCAGCGTGAAGTCC | |
| | AGCAGAGGCAGCAGTGATCCTCAGGGCGTTACATGTGGCGCC | |
| | GCTACACTGTCTGCCGAAAGAGTGCGGGGCGATAACAAAGAA | |
| | TACGAGTACAGCGTGGAATGCCAAGAGGACAGCGCCTGTCCA | |
| | GCCGCCGAAGAGTCTCTGCCTATCGAAGTGATGGTCGACGCC | |
| | GTGCACAAGCTGAAGTACGAGAACTACACCAGCAGCTTTTTC | |
| | ATCCGGGACATCATCAAGCCCGATCCTCCAAAGAACCTGCAG | |
| | CTGAAGCCTCTGAAGAACAGCAGACAGGTGGAAGTGTCCTGG | |
| | GAGTACCCCGACACCTGGTCTACACCCCACAGCTACTTCAGC | |
| | CTGACCTTTTGCGTGCAAGTGCAGGGCAAGTCCAAGCGCGAG | |
| | AAAAAGGACCGGGTGTTCACCGACAAGACCAGCGCCACCGTG | |
| | ATCTGCAGAAAGAACGCCAGCATCAGCGTCAGAGCCCAGGAC | |
| | CGGTACTACAGCAGCTCTTGGAGCGAATGGGCCAGCGTGCCA | |
| | TGTAGCGGAGGTGGTGGTAGCGGAGGCGGCGGAAGCGGCGGT | |
| | GGTGGATCAGGTGGTGGTGGCTCTAGAAACCTGCCAGTGGCT | |
| | ACCCCTGATCCTGGCATGTTCCCTTGTCTGCACCACAGCCAG | |
| | AACCTGCTGAGAGCCGTGTCCAACATGCTGCAGAAGGCCAGA | |
| | CAGACCCTGGAATTCTACCCCTGCACCAGCGAGGAAATCGAC | |
| | CACGAGGACATCACCAAGGATAAGACCAGCACCGTGGAAGCC | |
| | TGCCTGCCTCTGGAACTGACCAAGAACGAGAGCTGCCTGAAC | |
| | AGCCGGGAAACCTCCTTCATCACCAACGGCTCTTGCCTGGCC | |
| | AGCAGAAAGACAAGCTTCATGATGGCCCTGTGCCTGAGCAGC | |
| | ATCTACGAGGACCTGAAGATGTACCAGGTGGAATTCAAGACC | |
| | ATGAACGCCAAGCTGCTGATGGACCCCAAGCGGCAGATCTTC | |
| | CTGGACCAGAACATGCTGGCTGTGATCGACGAGCTGATGCAG | |
| | GCCCTGAACTTCAACAGCGAGACAGTGCCCCAGAAGTCTAGC | |
| | CTGGAAGAACCCGACTTCTACAAGACCAAGATCAAGCTGTGC | |
| | ATCCTGCTGCACGCCTTCCGGATCAGAGCCGTGACCATCGAC | |
| | AGAGTGATGAGCTACCTGAACGCCTCCTGA | |
| 3 | (G₄S)₃ | G₄S Linker |
| 4 | (EAAAK)₃ | EAAAK linker |
| 5 | (G₄S)ₙ | G₄S Linker |
| 6 | (EAAAK)ₙ | EAAAK linker |
| 7 | MMVDGDNSHVEMKLAVDEEENADNNTKANVTKPKRCSGSICY | TFRC_G4S_IL-2 |
| | GTIAVIVFFLIGFMIGYLGYCKSSDGPGETGGGGGSGGGGSG | |
| | GGGSAPTSSSTKKTQLQLEHLLLDLQMILNGINNYKNPKLTR | |
| | MLTFKFYMPKKATELKHLQCLEEELKPLEEVLNLAQSKNFHL | |
| | RPRDLISNINVIVLELKGSETTFMCEYADETATIVEFLNRWI | |
| | TFCQSIISTLT | |
| 8 | MLKKRGNHSTGLCLLVMFFMVLVALVGLGLGMFQLFHLQKET | FasL_G4S_IL-2 |
| | GGGGGSGGGGSGGGGSAPTSSSTKKTQLQLEHLLLDLQMILN | |
| | GINNYKNPKLTRMLTFKFYMPKKATELKHLQCLEEELKPLEE | |
| | VLNLAQSKNFHLRPRDLISNINVIVLELKGSETTFMCEYADE | |
| | TATIVEFLNRWITFCQSIISTLT | |
| 9 | MMVDGDNSHVEMKLAVDEEENADNNTKANVTKPKRCSGSICY | TFRC_G4S_IL-12 |
| | GTIAVIVFFLIGFMIGYLGYCKSSDGPGETGGGGGSGGGGSG | |
| | GGGSIWELKKDVYVVELDWYPDAPGEMVVLTCDTPEEDGITW | |
| | TLDQSSEVLGSGKTLTIQVKEFGDAGQYTCHKGGEVLSHSLL | |
| | LLHKKEDGIWSTDILKDQKEPKNKTFLRCEAKNYSGRFTCWW | |
| | LTTISTDLTFSVKSSRGSSDPQGVTCGAATLSAERVRGDNKE | |
| | YEYSVECQEDSACPAAEESLPIEVMVDAVHKLKYENYTSSFF | |
| | IRDIIKPDPPKNLQLKPLKNSRQVEVSWEYPDTWSTPHSYFS | |
| | LTFCVQVQGKSKREKKDRVFTDKTSATVICRKNASISVRAQD | |
| | RYYSSSWSEWASVPCSGGGGSGGGGSGGGGSGGGGSRNLPVA | |
| | TPDPGMFPCLHHSQNLLRAVSNMLQKARQTLEFYPCTSEEID | |
| | HEDITKDKTSTVEACLPLELTKNESCLNSRETSFITNGSCLA | |
| | SRKTSFMMALCLSSIYEDLKMYQVEFKTMNAKLLMDPKRQIF | |
| | LDQNMLAVIDELMQALNFNSETVPQKSSLEEPDFYKTKIKLC | |
| | ILLHAFRIRAVTIDRVMSYLNAS | |
| 10 | MMVDGDNSHVEMKLAVDEEENADNNTKANVTKPKRCSGSICY | TFRC_G4S_IFN- |
| | GTIAVIVFFLIGFMIGYLGYCKSSDGPGETGGGGGSGGGGSG | a2a |
| | GGGSCDLPQTHSLGSRRTLMLLAQMRKISLFSCLKDRHDFGF | |
| | PQEEFGNQFQKAETIPVLHEMIQQIFNLFSTKDSSAAWDETL | |
| | LDKFYTELYQQLNDLEACVIQGVGVTETPLMKEDSILAVRKY | |
| | FQRITLYLKEKKYSPCAWEVVRAEIMRSFSLSTNLQESLRSK | |
| | E | |
| 11 | MMVDGDNSHVEMKLAVDEEENADNNTKANVTKPKRCSGSICY | TFRC domain |
| | GTIAVIVFFLIGFMIGYLGYCKSSDGPGETG | |
| 12 | MLKKRGNHSTGLCLLVMFFMVLVALVGLGLGMFQLFHLQKET | FasL domain |
| | G | |
| 13 | ATGATGGTGGACGGCGACAACAGCCACGTGGAGATGAAGCTG | membrane-bound |
| | GCCGTGGACGAGGAGGAGAACGCCGACAACAACACCAAGGCC | IL-7 mRNA |
| | AACGTGACCAAGCCCAAGCGCTGCAGCGGCAGCATCTGCTAC | |
| | GGCACCATCGCCGTGATCGTGTTCTTCCTGATCGGCTTCATG | |
| | ATCGGCTACCTGGGCTACTGCAAGAGCAGCGACGGCCCCGGC | |
| | GAGACCGGCGGaGGCGGCGGaAGCGGCGGCGGaGGCAGCGGC | |
| | GGaGGCGGaAGCGACTGCGACATCGAGGGCAAGGACGGCAAG | |
| | CAGTACGAGAGCGTGCTGATGGTGAGCATCGACCAGCTGCTG | |
| | GACAGCATGAAGGAGATCGGCAGCAACTGCCTGAACAACGAG | |
| | TTCAACTTCTTCAAGCGCCACATCTGCGACGCCAACAAGGAG | |
| | GGCATGTTCCTGTTCCGCGCCGCCCGCAAGCTGCGCCAGTTC | |
| | CTGAAGATGAACAGCACCGGCGACTTCGACCTGCACCTGCTG | |
| | AAGGTGAGCGAGGGCACCACCATCCTGCTGAACTGCACCGGC | |
| | CAGGTGAAGGGCCGCAAGCCCGCCGCCCTGGGCGAGGCCCAG | |
| | CCCACCAAGAGCCTGGAGGAGAACAAGAGCCTGAAGGAGCAG | |
| | AAGAAGCTGAACGACCTGTGCTTCCTGAAGCGCCTGCTGCAG | |
| | GAGATCAAGACCTGCTGGAACAAGATCCTGATGGGCACCAAG | |
| | GAGCACtaa | |
| 18 | GGAAAUAAGAGAGAAAAGAAGAGUAAGAAGAAAUAUAAGAGC | E6 mRNA |
| | CACCAUGCACCAGAAACGGACCGCCAUGUUCCAGGAUCCUCA | |
| | AGAGAGGCCCAGAAAGCUGCCUCAGCUGUGUACCGAGCUGCA | |
| | GACCACCAUCCACGACAUCAUCCUGGAAUGCGUGUACUGCAA | |
| | GCAGCAGCUCCUGCGGAGAGAGGUGUACGAUUUCGCCUUCCG | |
| | GGACCUGUGCAUCGUGUACAGAGAUGGCAACCCCUACGCCGU | |
| | GUGCGACAAGUGCCUGAAGUUCUACAGCAAGAUCAGCGAGUA | |
| | CCGGCACUACUGCUACAGCCUGUACGGCACCACACUGGAACA | |
| | GCAGUACAACAAGCCCCUGUGCGACCUGCUGAUCCGGUGCAU | |
| | CAACUGCCAGAAACCUCUGUGCCCCGAGGAAAAGCAGCGGCA | |
| | CCUGGACAAGAAGCAGCGGUUCCACAACAUCAGAGGCCGGUG | |
| | GACCGGCAGAUGCAUGAGCUGUUGUCGGAGCAGCAGAACCAG | |
| | ACGGGAAACCCAGCUGUGAGCUGCCUUCUGCGGGGCUUGCCU | |
| | UCUGGCCAUGCCCUUCUUCUCUCCCUUGCACCUGUACCUCUU | |
| | GGUCUUUGAAUAAAGCCUGAGUAGGAAGUCUAGAAAAAAAAA | |
| | AAAAAAAAAAAAAAAAAAAAAAAAAAAAAAAAAAAAAAAAAA | |
| | AAAAAAAAAAAAAAAAAAAAAAAAAAAAA | |
| 19 | GGAAAUAAGAGAGAAAAGAAGAGUAAGAAGAAAUAUAAGAGC | E7 mRNA |
| | CACCAUGCACGGCGAUACCCCUACACUGCACGAGUACAUGCU | |
| | GGACCUGCAGCCUGAGACAACCGACCUGUACUGCUACGAGCA | |
| | GCUGAACGACAGCAGCGAGGAAGAGGACGAGAUUGACGGACC | |
| | UGCCGGACAGGCCGAACCUGAUAGAGCCCACUACAAUAUCGU | |
| | GACCUUCUGCUGCAAGUGCGACAGCACCCUGAGACUGUGUGU | |
| | GCAGAGCACCCACGUGGACAUCAGAACCCUGGAAGAUCUGCU | |
| | GAUGGGCACCCUGGGCAUCGUGUGCCCUAUCUGUAGCCAGAA | |
| | GCCUUGAGCUGCCUUCUGCGGGGCUUGCCUUCUGGCCAUGCC | |
| | CUUCUUCUCUCCCUUGCACCUGUACCUCUUGGUCUUUGAAUA | |
| | AAGCCUGAGUAGGAAGUCUAGAAAAAAAAAAAAAAAAAAAAA | |
| | AAAAAAAAAAAAAAAAAAAAAAAAAAAAAAAAAAAAAAAAAA | |
| | AAAAAAAAAAAAAAAAA | |
| 20 | GGAAAUAAGAGAGAAAAGAAGAGUAAGAAGAAAUAUAAGAGC | CD86 mRNA |
| | CACCAUGGAUCCCCAGUGUACCAUGGGCCUGAGCAACAUCCU | |
| | GUUCGUGAUGGCCUUUCUGCUGAGCGGAGCCGCUCCUCUGAA | |
| | GAUCCAGGCCUACUUCAACGAGACAGCCGACCUGCCUUGCCA | |
| | GUUCGCCAACAGCCAGAAUCAGAGCCUGAGCGAGCUGGUGGU | |
| | GUUCUGGCAGGACCAAGAGAACCUGGUGCUGAACGAGGUGUA | |
| | CCUGGGCAAAGAAAAGUUCGACAGCGUGCACAGCAAGUACAU | |
| | GGGCAGAACCAGCUUCGACUCCGACAGCUGGACACUGAGACU | |
| | GCACAACCUGCAGAUCAAGGACAAGGGCCUGUACCAGUGCAU | |
| | CAUCCACCACAAGAAACCCACCGGCAUGAUCAGAAUCCACCA | |
| | GAUGAACAGCGAGCUGAGCGUGCUGGCCAACUUCAGCCAGCC | |
| | UGAGAUCGUGCCCAUCAGCAACAUCACCGAGAACGUGUACAU | |
| | CAACCUGACCUGCAGCAGCAUCCACGGCUACCCCGAGCCUAA | |
| | GAAAAUGUCCGUGCUGCUGCGGACCAAGAACAGCACCAUCGA | |
| | GUACGACGGCGUGAUGCAGAAAAGCCAGGACAACGUGACCGA | |
| | GCUGUACGACGUGUCCAUCAGCCUGUCCGUGUCAUUCCCCGA | |
| | CGUGACCAGCAAUAUGACCAUCUUCUGCAUCCUGGAAACCGA | |
| | CAAGACCCGGCUGCUGUCUAGCCCCUUCAGCAUCGAGCUGGA | |
| | AGAUCCCCAGCCUCCUCCUGAUCACAUCCCUUGGAUUACCGC | |
| | CGUGCUGCCCACCGUGAUCAUCUGCGUGAUGGUGUUUUGCCU | |
| | GAUCCUGUGGAAGUGGAAGAAGAAGAAACGGCCUCGGAACAG | |
| | CUACAAGUGCGGCACCAACACCAUGGAACGGGAAGAGAGCGA | |
| | GCAGACCAAGAAGAGAGAGAAGAUUCACAUCCCCGAGCGGAG | |
| | CGACGAAGCCCAGAGAGUGUUUAAGAGCAGCAAGACCAGCAG | |
| | CUGCGACAAGAGCGAUACCUGCUUCUGAGCUGCCUUCUGCGG | |
| | GGCUUGCCUUCUGGCCAUGCCCUUCUUCUCUCCCUUGCACCU | |
| | GUACCUCUUGGUCUUUGAAUAAAGCCUGAGUAGGAAGUCUAG | |
| | AAAAAAAAAAAAAAAAAAAAAAAAAAAAAAAAAAAAAAAAAA | |
| | AAAAAAAAAAAAAAAAAAAAAAAAAAAAAAAAAAAAAA | |
| 21 | GGAAAUAAGAGAGAAAAGAAGAGUAAGAAGAAAUAUAAGAGC | membrane-bound |
| | CACCAUGAUGGUGGACGGCGACAACAGCCACGUGGAAAUGAA | IL-2 mRNA |
| | GCUGGCCGUGGACGAGGAAGAGAACGCCGACAACAACACCAA | |
| | GGCCAACGUGACCAAGCCUAAGAGAUGCAGCGGCAGCAUCUG | |
| | CUACGGCACAAUCGCCGUGAUCGUGUUCUUCCUGAUCGGCUU | |
| | UAUGAUCGGCUACCUGGGCUACUGCAAGAGCAGUGAUGGACC | |
| | UGGCGAAACAGGCGGAGGCGGAGGAUCUGGUGGCGGAGGAAG | |
| | CGGUGGCGGCGGUUCUGCUCCUACAAGCAGCAGCACCAAGAA | |
| | AACCCAGCUGCAACUGGAACAUCUGCUGCUGGACCUGCAGAU | |
| | GAUCCUGAACGGCAUCAACAACUACAAGAACCCCAAGCUGAC | |
| | CCGGAUGCUGACCUUCAAGUUCUACAUGCCCAAGAAGGCCAC | |
| | CGAGCUGAAGCACCUCCAGUGCCUGGAAGAGGAACUGAAGCC | |
| | CCUGGAAGAAGUGCUGAAUCUGGCCCAGAGCAAGAACUUCCA | |
| | CCUGAGGCCUAGGGACCUGAUCAGCAACAUCAAUGUGAUCGU | |
| | GCUGGAACUCAAGGGCAGCGAGACAACCUUCAUGUGCGAGUA | |
| | CGCCGACGAGACAGCUACCAUCGUGGAAUUUCUGAACCGGUG | |
| | GAUCACCUUCUGCCAGAGCAUCAUCAGCACCCUGACCUGAGC | |
| | UGCCUUCUGCGGGGCUUGCCUUCUGGCCAUGCCCUUCUUCUC | |
| | UCCCUUGCACCUGUACCUCUUGGUCUUUGAAUAAAGCCUGAG | |
| | UAGGAAGUCUAGAAAAAAAAAAAAAAAAAAAAAAAAAAAAAA | |
| | AAAAAAAAAAAAAAAAAAAAAAAAAAAAAAAAAAAAAAAAAA | |
| | AAAAAAAA | |
| 22 | GGAAAUAAGAGAGAAAAGAAGAGUAAGAAGAAAUAUAAGAGC | membrane-bound |
| | CACCAUGAUGGUGGACGGCGACAACAGCCACGUGGAAAUGAA | IL-12 mRNA |
| | GCUGGCCGUGGACGAGGAAGAGAACGCCGACAACAACACCAA | |
| | GGCCAACGUGACCAAGCCUAAGAGAUGCAGCGGCAGCAUCUG | |
| | CUACGGCACAAUCGCCGUGAUCGUGUUCUUCCUGAUCGGCUU | |
| | UAUGAUCGGCUACCUGGGCUACUGCAAGAGCAGUGAUGGACC | |
| | UGGCGAAACAGGCGGAGGCGGAGGAUCUGGUGGCGGAGGAAG | |
| | UGGCGGCGGAGGUUCUAUUUGGGAGCUGAAGAAAGACGUGUA | |
| | CGUGGUGGAACUGGACUGGUAUCCCGAUGCUCCUGGCGAGAU | |
| | GGUGGUGCUGACCUGCGAUACCCCUGAAGAGGACGGCAUCAC | |
| | CUGGACACUGGAUCAGUCUAGCGAGGUGCUCGGCAGCGGCAA | |
| | GACCCUGACCAUCCAAGUGAAAGAGUUUGGCGACGCCGGCCA | |
| | GUACACCUGUCACAAAGGCGGAGAAGUGCUGAGCCACAGCCU | |
| | GCUGCUGCUCCACAAGAAAGAGGAUGGCAUUUGGAGCACCGA | |
| | CAUCCUGAAGGACCAGAAAGAGCCCAAGAACAAGACCUUCCU | |
| | GAGAUGCGAGGCCAAGAACUACAGCGGCCGGUUCACAUGUUG | |
| | GUGGCUGACCACCAUCAGCACCGACCUGACCUUCAGCGUGAA | |
| | GUCCAGCAGAGGCAGCAGUGAUCCUCAGGGCGUUACAUGUGG | |
| | CGCCGCUACACUGUCUGCCGAAAGAGUGCGGGGCGAUAACAA | |
| | AGAAUACGAGUACAGCGUGGAAUGCCAAGAGGACAGCGCCUG | |
| | UCCAGCCGCCGAAGAGUCUCUGCCUAUCGAAGUGAUGGUCGA | |
| | CGCCGUGCACAAGCUGAAGUACGAGAACUACACCAGCAGCUU | |
| | UUUCAUCCGGGACAUCAUCAAGCCCGAUCCUCCAAAGAACCU | |
| | GCAGCUGAAGCCUCUGAAGAACAGCAGACAGGUGGAAGUGUC | |
| | CUGGGAGUACCCCGACACCUGGUCUACACCCCACAGCUACUU | |
| | CAGCCUGACCUUUUGCGUGCAAGUGCAGGGCAAGUCCAAGCG | |
| | CGAGAAAAAGGACCGGGUGUUCACCGACAAGACCAGCGCCAC | |
| | CGUGAUCUGCAGAAAGAACGCCAGCAUCAGCGUCAGAGCCCA | |
| | GGACCGGUACUACAGCAGCUCUUGGAGCGAAUGGGCCAGCGU | |
| | GCCAUGUAGCGGAGGUGGUGGUAGCGGAGGCGGCGGAAGCGG | |
| | CGGUGGUGGAUCAGGUGGUGGUGGCUCUAGAAACCUGCCAGU | |
| | GGCUACCCCUGAUCCUGGCAUGUUCCCUUGUCUGCACCACAG | |
| | CCAGAACCUGCUGAGAGCCGUGUCCAACAUGCUGCAGAAGGC | |
| | CAGACAGACCCUGGAAUUCUACCCCUGCACCAGCGAGGAAAU | |
| | CGACCACGAGGACAUCACCAAGGAUAAGACCAGCACCGUGGA | |
| | AGCCUGCCUGCCUCUGGAACUGACCAAGAACGAGAGCUGCCU | |
| | GAACAGCCGGGAAACCUCCUUCAUCACCAACGGCUCUUGCCU | |
| | GGCCAGCAGAAAGACAAGCUUCAUGAUGGCCCUGUGCCUGAG | |
| | CAGCAUCUACGAGGACCUGAAGAUGUACCAGGUGGAAUUCAA | |
| | GACCAUGAACGCCAAGCUGCUGAUGGACCCCAAGCGGCAGAU | |
| | CUUCCUGGACCAGAACAUGCUGGCUGUGAUCGACGAGCUGAU | |
| | GCAGGCCCUGAACUUCAACAGCGAGACAGUGCCCCAGAAGUC | |
| | UAGCCUGGAAGAACCCGACUUCUACAAGACCAAGAUCAAGCU | |
| | GUGCAUCCUGCUGCACGCCUUCCGGAUCAGAGCCGUGACCAU | |
| | CGACAGAGUGAUGAGCUACCUGAACGCCUCCUGAGCUGCCUU | |
| | CUGCGGGGCUUGCCUUCUGGCCAUGCCCUUCUUCUCUCCCUU | |
| | GCACCUGUACCUCUUGGUCUUUGAAUAAAGCCUGAGUAGGAA | |
| | GUCUAGAAAAAAAAAAAAAAAAAAAAAAAAAAAAAAAAAAAA | |
| | AAAAAAAAAAAAAAAAAAAAAAAAAAAAAAAAAAAAAAAAAA | |
| | AA | |

1-35. (canceled)

36. A method for treating a HPV+ recurrent, locally advanced, or metastatic tumor in a subject in need thereof, the method comprising administering to the subject a composition comprising modified antigen presenting cells (“enhanced APCs”), wherein the enhanced APCs comprise an HPV antigen and are capable of activating T cells in an HLA agnostic manner.

37. The method of claim 36, wherein the enhanced APCs comprise T cells, B cells, NK cells, monocytes, or combinations thereof.

38-44. (canceled)

45. The method of claim 36, wherein the enhanced APCs exhibit increased expression of a co-stimulatory molecule and/or a cytokine as compared to corresponding non-modified APCs (“reference APCs”).

46. The method of claim 45, wherein the co-stimulatory molecule comprises CD86 and/or wherein the cytokine comprises a membrane-bound cytokine.

47-48. (canceled)

49. The method of claim 45, wherein the cytokine comprises IL-2, IL-12, or both.

50. The method of claim 36, wherein the enhanced APCs have been prepared by passing a cell suspension comprising input APCs through a cell-deforming constriction, thereby causing perturbations in the APCs such that a nucleic acid encoding the antigen, a nucleic acid encoding the co-stimulatory molecule and/or a nucleic acid encoding the cytokine enters the APCs through the perturbations when contacted with the APCs, and thereby producing the enhanced APCs.

51. (canceled)

52. The method of claim 50, wherein the nucleic acid encoding the antigen, the nucleic acid encoding the co-stimulatory molecule, and/or the nucleic acid encoding the cytokine is an mRNA.

53. (canceled)

54. The method of claim 36, wherein the enhanced APCs have been conditioned in a medium comprising an adjuvant.

55. The method of claim 54, wherein the enhanced APCs have been conditioned in a medium comprising an adjuvant:
(i) for about 2 hours to about 10 hours;
(ii) for about 3 hours to about 6 hours;
(iii) for about 4 hours; or
(iv) at about 37° C.

56-60. (canceled)

61. The method of claim 36, wherein the composition comprising enhanced APCs is administered in combination with an immune checkpoint inhibitor, wherein the one or more immune checkpoint inhibitors comprises an antagonist of CTLA-4 and/or an antagonist of PD-1/PD-L1, wherein the antagonist of PD-1/PD-L1 is an antibody that binds PD-1 or an antibody that binds PD-L1 and wherein the antagonist of CTLA-4 is an antibody that binds CTLA-4.

62-65. (canceled)

66. The method of claim 61, wherein the antibody that binds PD-1 is pembrolizumab or nivolumab, wherein the antibody that binds PD-L1 is atezolizumab and wherein the antibody that binds CTLA-4 is ipilimumab.

67-71. (canceled)

72. The method of claim 36, wherein the HPV-associated cancer and/or HPV+tumor comprises a cervical cancer, anal cancer, vulval cancer, vaginal cancer, penile cancer, or oropharyngeal cancer.

73. The method of claim 36, wherein the composition comprising enhanced APCs is administered in an amount of:
(i) about 0.25×10⁶ cells/kg to about 7.50×10⁶ cells/kg;
(ii) about 0.5×10⁶ cells/kg to about 5.0×10⁶ cells/kg;
(iii) about 0.5×10⁶ cells/kg;
(iv) about 2.5×10⁶ cells/kg:
(v) 5.0×10⁶ cells/kg;
(vi) 0.25×10⁶ cells/kg;
(vii) 1.25×10⁶ cells/kg;
(viii) 3.25×10⁶ cells/kg; or
(ix) 7.5×10⁶ cells/kg.

74-84. (canceled)

85. The method of claim 66, wherein:
(i) pembrolizumab is administered in an amount of about 10 mg to about 400 mg;
(ii) nivolumab is administered in an amount of about 15 mg to about 500 mg;
(iii) atezolizumab is administered in an amount of about 75 mg to about 1700 mg; or
(iv) ipilimumab is administered in an amount of about 1 mg/kg to about 10 mg/kg.

86-95. (canceled)

96. The method of claim 36, wherein the composition comprising enhanced APCs is administered on day 1 of a three-week cycle and/or day 2 of a first three-week cycle.

97-99. (canceled)

100. The method of claim 61, wherein the antibody that binds PD-1, the antibody that binds PD-L1, and/or the antibody that binds CTLA 4 is administered once per three-week cycle.

101. The method of claim 61, wherein:
(i) the antibody that binds PD-1 is administered on:
(a) day 1 of a three-week cycle;
(b) day 8 of the first three-week cycle;
(c) day 8 of the first three-week cycle and day 1 of each subsequent three-week cycle;
(d) day 1 of the third three-week cycle; or
(e) day 1 of the third three-week cycle and day 1 of each subsequent three-week cycle;
(ii) the antibody that binds PD-L1 is administered on day 8 of the first three-week cycle and day 1 of each subsequent cycle; or
(iii) the antibody that binds CTLA-4 is administered on day 1 of each three-week cycle or once per two three-week cycles.

102-105. (canceled)

106. The method of claim 100, wherein:
(i) the antibody that binds PD-1 is pembrolizumab, wherein the pembrolizumab is administered in an amount of about 200 mg;
(ii) the antibody that binds PD-1 is nivolumab, wherein the nivolumab is administered in an amount of about 360 mg;
(iii) the antibody that binds CTLA-4 is ipilimumab, wherein the ipilimumab is administered at a dose of about 3 mg/kg; or
(iv) the antibody that binds PD-L1 is atezolizumab, wherein the atezolizumab is administered at a dose of about 1200 mg.

107-113. (canceled)

114. The method of claim 36, wherein the composition comprising enhanced APCs comprises:
(a) about 5×10⁶ enhanced APCs to about 1×10⁹ enhanced APCs,
(b) a cryopreservation medium at a concentration of about 40% to about 95% (w/w),
(c) a hypothermic preservation medium at a concentration of about 25% to about 35% (w/w), and
(d) a human serum albumin solution at a concentration of 15% to about 25% (w/w), wherein the pH of the composition is about pH 6.0 to about pH 8.5.

115-119. (canceled)

120. The method of claim 114, wherein the cryopreservation medium is CryoStor® CS10 and wherein the hypothermic preservation medium is HypoThermasol® FRS.

121-137. (canceled)


SEQUENCE LISTING (22 sequences)

SEQ ID NO: 1 - protein, 762 aa, synthetic construct
  source 1..762: note = TFRC_G4S_IFN-a2a mRNA
ATGATGGTGGACGGCGACAACAGCCACGTGGAAATGAAGCTGGCCGTGGACGAGGAAGAGAACGCCGACAACAACACCAAGGCCAACGTGACCAAGCCTAAGAGATGCAGCGGCAGCATCTGCTACGGCACAATCGCCGTGATCGTGTTCTTCCTGATCGGCTTTATGATCGGCTACCTGGGCTACTGCAAGAGCAGTGATGGACCTGGCGAAACAGGCGGAGGCGGAGGATCTGGTGGCGGAGGAAGCGGTGGCGGCGGATCTTGTGATCTGCCTCAGACACACAGCCTGGGCAGCAGACGAACACTGATGCTGCTGGCCCAGATGCGGAAGATCAGCCTGTTCAGCTGCCTGAAGGACCGGCACGATTTCGGCTTCCCTCAAGAGGAATTCGGCAACCAGTTCCAGAAGGCCGAGACAATCCCTGTGCTGCACGAGATGATCCAGCAGATCTTCAACCTGTTCTCCACCAAGGACAGCAGCGCCGCCTGGGATGAGACACTGCTGGACAAGTTCTACACCGAGCTGTACCAGCAGCTGAATGACCTGGAAGCCTGCGTGATCCAAGGCGTGGGAGTGACAGAGACACCCCTGATGAAGGAAGATAGCATCCTGGCCGTGCGCAAGTACTTCCAGCGGATCACCCTGTACCTGAAAGAGAAGAAGTACAGCCCCTGCGCCTGGGAAGTCGTGCGGGCCGAAATCATGAGAAGCTTCAGCCTGAGCACCAACCTGCAAGAGAGCCTGCGGAGCAAAGAGTGA

SEQ ID NO: 2 - protein, 1836 aa, synthetic construct
  source 1..1836: note = TFRC_G4S_IL-12 mRNA
ATGATGGTGGACGGCGACAACAGCCACGTGGAAATGAAGCTGGCCGTGGACGAGGAAGAGAACGCCGACAACAACACCAAGGCCAACGTGACCAAGCCTAAGAGATGCAGCGGCAGCATCTGCTACGGCACAATCGCCGTGATCGTGTTCTTCCTGATCGGCTTTATGATCGGCTACCTGGGCTACTGCAAGAGCAGTGATGGACCTGGCGAAACAGGCGGAGGCGGAGGATCTGGTGGCGGAGGAAGTGGCGGCGGAGGTTCTATTTGGGAGCTGAAGAAAGACGTGTACGTGGTGGAACTGGACTGGTATCCCGATGCTCCTGGCGAGATGGTGGTGCTGACCTGCGATACCCCTGAAGAGGACGGCATCACCTGGACACTGGATCAGTCTAGCGAGGTGCTCGGCAGCGGCAAGACCCTGACCATCCAAGTGAAAGAGTTTGGCGACGCCGGCCAGTACACCTGTCACAAAGGCGGAGAAGTGCTGAGCCACAGCCTGCTGCTGCTCCACAAGAAAGAGGATGGCATTTGGAGCACCGACATCCTGAAGGACCAGAAAGAGCCCAAGAACAAGACCTTCCTGAGATGCGAGGCCAAGAACTACAGCGGCCGGTTCACATGTTGGTGGCTGACCACCATCAGCACCGACCTGACCTTCAGCGTGAAGTCCAGCAGAGGCAGCAGTGATCCTCAGGGCGTTACATGTGGCGCCGCTACACTGTCTGCCGAAAGAGTGCGGGGCGATAACAAAGAATACGAGTACAGCGTGGAATGCCAAGAGGACAGCGCCTGTCCAGCCGCCGAAGAGTCTCTGCCTATCGAAGTGATGGTCGACGCCGTGCACAAGCTGAAGTACGAGAACTACACCAGCAGCTTTTTCATCCGGGACATCATCAAGCCCGATCCTCCAAAGAACCTGCAGCTGAAGCCTCTGAAGAACAGCAGACAGGTGGAAGTGTCCTGGGAGTACCCCGACACCTGGTCTACACCCCACAGCTACTTCAGCCTGACCTTTTGCGTGCAAGTGCAGGGCAAGTCCAAGCGCGAGAAAAAGGACCGGGTGTTCACCGACAAGACCAGCGCCACCGTGATCTGCAGAAAGAACGCCAGCATCAGCGTCAGAGCCCAGGACCGGTACTACAGCAGCTCTTGGAGCGAATGGGCCAGCGTGCCATGTAGCGGAGGTGGTGGTAGCGGAGGCGGCGGAAGCGGCGGTGGTGGATCAGGTGGTGGTGGCTCTAGAAACCTGCCAGTGGCTACCCCTGATCCTGGCATGTTCCCTTGTCTGCACCACAGCCAGAACCTGCTGAGAGCCGTGTCCAACATGCTGCAGAAGGCCAGACAGACCCTGGAATTCTACCCCTGCACCAGCGAGGAAATCGACCACGAGGACATCACCAAGGATAAGACCAGCACCGTGGAAGCCTGCCTGCCTCTGGAACTGACCAAGAACGAGAGCTGCCTGAACAGCCGGGAAACCTCCTTCATCACCAACGGCTCTTGCCTGGCCAGCAGAAAGACAAGCTTCATGATGGCCCTGTGCCTGAGCAGCATCTACGAGGACCTGAAGATGTACCAGGTGGAATTCAAGACCATGAACGCCAAGCTGCTGATGGACCCCAAGCGGCAGATCTTCCTGGACCAGAACATGCTGGCTGTGATCGACGAGCTGATGCAGGCCCTGAACTTCAACAGCGAGACAGTGCCCCAGAAGTCTAGCCTGGAAGAACCCGACTTCTACAAGACCAAGATCAAGCTGTGCATCCTGCTGCACGCCTTCCGGATCAGAGCCGTGACCATCGACAGAGTGATGAGCTACCTGAACGCCTCCTGA

SEQ ID NO: 3 - protein, 15 aa, synthetic construct
  source 1..15: note = G4S Linker
GGGGSGGGGSGGGGS

SEQ ID NO: 4 - protein, 15 aa, synthetic construct
  source 1..15: note = EAAAK linker
EAAAKEAAAKEAAAK

SEQ ID NO: 5 - protein, 5 aa, synthetic construct
  source 1..5: note = G4S Linker
  REGION 1..5: note = The sequence can comprise 1-100 repeats of the GGGGS subsequence
GGGGS

SEQ ID NO: 6 - protein, 5 aa, synthetic construct
  source 1..5: note = EAAAK linker
  REGION 1..5: note = The entire amino acid sequence can be repeated 1-100 times
EAAAK

SEQ ID NO: 7 - protein, 221 aa, synthetic construct
  source 1..221: note = TFRC_G4S_IL-2
MMVDGDNSHVEMKLAVDEEENADNNTKANVTKPKRCSGSICYGTIAVIVFFLIGFMIGYLGYCKSSDGPGETGGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 8 - protein, 191 aa, synthetic construct
  source 1..191: note = FasL_G4S_IL-2
MLKKRGNHSTGLCLLVMFFMVLVALVGLGLGMFQLFHLQKETGGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 9 - protein, 611 aa, synthetic construct
  source 1..611: note = TFRC_G4S_IL-12
MMVDGDNSHVEMKLAVDEEENADNNTKANVTKPKRCSGSICYGTIAVIVFFLIGFMIGYLGYCKSSDGPGETGGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 10 - protein, 253 aa, synthetic construct
  source 1..253: note = TFRC_G4S_IFN-a2a
MMVDGDNSHVEMKLAVDEEENADNNTKANVTKPKRCSGSICYGTIAVIVFFLIGFMIGYLGYCKSSDGPGETGGGGGSGGGGSGGGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 11 - protein, 73 aa, synthetic construct
  source 1..73: note = TFRC domain
MMVDGDNSHVEMKLAVDEEENADNNTKANVTKPKRCSGSICYGTIAVIVFFLIGFMIGYLGYCKSSDGPGETG

SEQ ID NO: 12 - protein, 43 aa, synthetic construct
  source 1..43: note = FasL domain
MLKKRGNHSTGLCLLVMFFMVLVALVGLGLGMFQLFHLQKETG

SEQ ID NO: 13 - protein, 723 aa, synthetic construct
  source 1..723: note = membrane-bound IL-7 mRNA
ATGATGGTGGACGGCGACAACAGCCACGTGGAGATGAAGCTGGCCGTGGACGAGGAGGAGAACGCCGACAACAACACCAAGGCCAACGTGACCAAGCCCAAGCGCTGCAGCGGCAGCATCTGCTACGGCACCATCGCCGTGATCGTGTTCTTCCTGATCGGCTTCATGATCGGCTACCTGGGCTACTGCAAGAGCAGCGACGGCCCCGGCGAGACCGGCGGAGGCGGCGGAAGCGGCGGCGGAGGCAGCGGCGGAGGCGGAAGCGACTGCGACATCGAGGGCAAGGACGGCAAGCAGTACGAGAGCGTGCTGATGGTGAGCATCGACCAGCTGCTGGACAGCATGAAGGAGATCGGCAGCAACTGCCTGAACAACGAGTTCAACTTCTTCAAGCGCCACATCTGCGACGCCAACAAGGAGGGCATGTTCCTGTTCCGCGCCGCCCGCAAGCTGCGCCAGTTCCTGAAGATGAACAGCACCGGCGACTTCGACCTGCACCTGCTGAAGGTGAGCGAGGGCACCACCATCCTGCTGAACTGCACCGGCCAGGTGAAGGGCCGCAAGCCCGCCGCCCTGGGCGAGGCCCAGCCCACCAAGAGCCTGGAGGAGAACAAGAGCCTGAAGGAGCAGAAGAAGCTGAACGACCTGTGCTTCCTGAAGCGCCTGCTGCAGGAGATCAAGACCTGCTGGAACAAGATCCTGATGGGCACCAAGGAGCACTAA

SEQ ID NO: 14 - protein, 158 aa, synthetic construct
  source 1..158: note = HPV-16 E6 UniProt: P03126
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 15 - protein, 98 aa, synthetic construct
  source 1..98: note = HPV-16 E7 UniProt: P03129
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 16 - protein, 158 aa, synthetic construct
  source 1..158: note = HPV-18 E6 UniProt: P06463
MARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQV

SEQ ID NO: 17 - protein, 105 aa, synthetic construct
  source 1..105: note = HPV-18 E7 UniProt: P06788
MHGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQ

SEQ ID NO: 18 - protein, 701 aa, synthetic construct
  source 1..701: note = E6 mRNA
GGAAAUAAGAGAGAAAAGAAGAGUAAGAAGAAAUAUAAGAGCCACCAUGCACCAGAAACGGACCGCCAUGUUCCAGGAUCCUCAAGAGAGGCCCAGAAAGCUGCCUCAGCUGUGUACCGAGCUGCAGACCACCAUCCACGACAUCAUCCUGGAAUGCGUGUACUGCAAGCAGCAGCUCCUGCGGAGAGAGGUGUACGAUUUCGCCUUCCGGGACCUGUGCAUCGUGUACAGAGAUGGCAACCCCUACGCCGUGUGCGACAAGUGCCUGAAGUUCUACAGCAAGAUCAGCGAGUACCGGCACUACUGCUACAGCCUGUACGGCACCACACUGGAACAGCAGUACAACAAGCCCCUGUGCGACCUGCUGAUCCGGUGCAUCAACUGCCAGAAACCUCUGUGCCCCGAGGAAAAGCAGCGGCACCUGGACAAGAAGCAGCGGUUCCACAACAUCAGAGGCCGGUGGACCGGCAGAUGCAUGAGCUGUUGUCGGAGCAGCAGAACCAGACGGGAAACCCAGCUGUGAGCUGCCUUCUGCGGGGCUUGCCUUCUGGCCAUGCCCUUCUUCUCUCCCUUGCACCUGUACCUCUUGGUCUUUGAAUAAAGCCUGAGUAGGAAGUCUAGAAAAAAAAAAAAAAAAAAAAAAAAAAAAAAAAAAAAAAAAAAAAAAAAAAAAAAAAAAAAAAAAAAAAAAAAAAAAAAAA

SEQ ID NO: 19 - protein, 521 aa, synthetic construct
  source 1..521: note = E7 mRNA
GGAAAUAAGAGAGAAAAGAAGAGUAAGAAGAAAUAUAAGAGCCACCAUGCACGGCGAUACCCCUACACUGCACGAGUACAUGCUGGACCUGCAGCCUGAGACAACCGACCUGUACUGCUACGAGCAGCUGAACGACAGCAGCGAGGAAGAGGACGAGAUUGACGGACCUGCCGGACAGGCCGAACCUGAUAGAGCCCACUACAAUAUCGUGACCUUCUGCUGCAAGUGCGACAGCACCCUGAGACUGUGUGUGCAGAGCACCCACGUGGACAUCAGAACCCUGGAAGAUCUGCUGAUGGGCACCCUGGGCAUCGUGUGCCCUAUCUGUAGCCAGAAGCCUUGAGCUGCCUUCUGCGGGGCUUGCCUUCUGGCCAUGCCCUUCUUCUCUCCCUUGCACCUGUACCUCUUGGUCUUUGAAUAAAGCCUGAGUAGGAAGUCUAGAAAAAAAAAAAAAAAAAAAAAAAAAAAAAAAAAAAAAAAAAAAAAAAAAAAAAAAAAAAAAAAAAAAAAAAAAAAAAAAA

SEQ ID NO: 20 - protein, 1214 aa, synthetic construct
  source 1..1214: note = CD86 mRNA
GGAAAUAAGAGAGAAAAGAAGAGUAAGAAGAAAUAUAAGAGCCACCAUGGAUCCCCAGUGUACCAUGGGCCUGAGCAACAUCCUGUUCGUGAUGGCCUUUCUGCUGAGCGGAGCCGCUCCUCUGAAGAUCCAGGCCUACUUCAACGAGACAGCCGACCUGCCUUGCCAGUUCGCCAACAGCCAGAAUCAGAGCCUGAGCGAGCUGGUGGUGUUCUGGCAGGACCAAGAGAACCUGGUGCUGAACGAGGUGUACCUGGGCAAAGAAAAGUUCGACAGCGUGCACAGCAAGUACAUGGGCAGAACCAGCUUCGACUCCGACAGCUGGACACUGAGACUGCACAACCUGCAGAUCAAGGACAAGGGCCUGUACCAGUGCAUCAUCCACCACAAGAAACCCACCGGCAUGAUCAGAAUCCACCAGAUGAACAGCGAGCUGAGCGUGCUGGCCAACUUCAGCCAGCCUGAGAUCGUGCCCAUCAGCAACAUCACCGAGAACGUGUACAUCAACCUGACCUGCAGCAGCAUCCACGGCUACCCCGAGCCUAAGAAAAUGUCCGUGCUGCUGCGGACCAAGAACAGCACCAUCGAGUACGACGGCGUGAUGCAGAAAAGCCAGGACAACGUGACCGAGCUGUACGACGUGUCCAUCAGCCUGUCCGUGUCAUUCCCCGACGUGACCAGCAAUAUGACCAUCUUCUGCAUCCUGGAAACCGACAAGACCCGGCUGCUGUCUAGCCCCUUCAGCAUCGAGCUGGAAGAUCCCCAGCCUCCUCCUGAUCACAUCCCUUGGAUUACCGCCGUGCUGCCCACCGUGAUCAUCUGCGUGAUGGUGUUUUGCCUGAUCCUGUGGAAGUGGAAGAAGAAGAAACGGCCUCGGAACAGCUACAAGUGCGGCACCAACACCAUGGAACGGGAAGAGAGCGAGCAGACCAAGAAGAGAGAGAAGAUUCACAUCCCCGAGCGGAGCGACGAAGCCCAGAGAGUGUUUAAGAGCAGCAAGACCAGCAGCUGCGACAAGAGCGAUACCUGCUUCUGAGCUGCCUUCUGCGGGGCUUGCCUUCUGGCCAUGCCCUUCUUCUCUCCCUUGCACCUGUACCUCUUGGUCUUUGAAUAAAGCCUGAGUAGGAAGUCUAGAAAAAAAAAAAAAAAAAAAAAAAAAAAAAAAAAAAAAAAAAAAAAAAAAAAAAAAAAAAAAAAAAAAAAAAAAAAAAAAA

SEQ ID NO: 21 - protein, 890 aa, synthetic construct
  source 1..890: note = membrane-bound IL-2 mRNA
GGAAAUAAGAGAGAAAAGAAGAGUAAGAAGAAAUAUAAGAGCCACCAUGAUGGUGGACGGCGACAACAGCCACGUGGAAAUGAAGCUGGCCGUGGACGAGGAAGAGAACGCCGACAACAACACCAAGGCCAACGUGACCAAGCCUAAGAGAUGCAGCGGCAGCAUCUGCUACGGCACAAUCGCCGUGAUCGUGUUCUUCCUGAUCGGCUUUAUGAUCGGCUACCUGGGCUACUGCAAGAGCAGUGAUGGACCUGGCGAAACAGGCGGAGGCGGAGGAUCUGGUGGCGGAGGAAGCGGUGGCGGCGGUUCUGCUCCUACAAGCAGCAGCACCAAGAAAACCCAGCUGCAACUGGAACAUCUGCUGCUGGACCUGCAGAUGAUCCUGAACGGCAUCAACAACUACAAGAACCCCAAGCUGACCCGGAUGCUGACCUUCAAGUUCUACAUGCCCAAGAAGGCCACCGAGCUGAAGCACCUCCAGUGCCUGGAAGAGGAACUGAAGCCCCUGGAAGAAGUGCUGAAUCUGGCCCAGAGCAAGAACUUCCACCUGAGGCCUAGGGACCUGAUCAGCAACAUCAAUGUGAUCGUGCUGGAACUCAAGGGCAGCGAGACAACCUUCAUGUGCGAGUACGCCGACGAGACAGCUACCAUCGUGGAAUUUCUGAACCGGUGGAUCACCUUCUGCCAGAGCAUCAUCAGCACCCUGACCUGAGCUGCCUUCUGCGGGGCUUGCCUUCUGGCCAUGCCCUUCUUCUCUCCCUUGCACCUGUACCUCUUGGUCUUUGAAUAAAGCCUGAGUAGGAAGUCUAGAAAAAAAAAAAAAAAAAAAAAAAAAAAAAAAAAAAAAAAAAAAAAAAAAAAAAAAAAAAAAAAAAAAAAAAAAAAAAAAA

SEQ ID NO: 22 - protein, 2060 aa, synthetic construct
  source 1..2060: note = membrane-bound IL-12 mRNA
GGAAAUAAGAGAGAAAAGAAGAGUAAGAAGAAAUAUAAGAGCCACCAUGAUGGUGGACGGCGACAACAGCCACGUGGAAAUGAAGCUGGCCGUGGACGAGGAAGAGAACGCCGACAACAACACCAAGGCCAACGUGACCAAGCCUAAGAGAUGCAGCGGCAGCAUCUGCUACGGCACAAUCGCCGUGAUCGUGUUCUUCCUGAUCGGCUUUAUGAUCGGCUACCUGGGCUACUGCAAGAGCAGUGAUGGACCUGGCGAAACAGGCGGAGGCGGAGGAUCUGGUGGCGGAGGAAGUGGCGGCGGAGGUUCUAUUUGGGAGCUGAAGAAAGACGUGUACGUGGUGGAACUGGACUGGUAUCCCGAUGCUCCUGGCGAGAUGGUGGUGCUGACCUGCGAUACCCCUGAAGAGGACGGCAUCACCUGGACACUGGAUCAGUCUAGCGAGGUGCUCGGCAGCGGCAAGACCCUGACCAUCCAAGUGAAAGAGUUUGGCGACGCCGGCCAGUACACCUGUCACAAAGGCGGAGAAGUGCUGAGCCACAGCCUGCUGCUGCUCCACAAGAAAGAGGAUGGCAUUUGGAGCACCGACAUCCUGAAGGACCAGAAAGAGCCCAAGAACAAGACCUUCCUGAGAUGCGAGGCCAAGAACUACAGCGGCCGGUUCACAUGUUGGUGGCUGACCACCAUCAGCACCGACCUGACCUUCAGCGUGAAGUCCAGCAGAGGCAGCAGUGAUCCUCAGGGCGUUACAUGUGGCGCCGCUACACUGUCUGCCGAAAGAGUGCGGGGCGAUAACAAAGAAUACGAGUACAGCGUGGAAUGCCAAGAGGACAGCGCCUGUCCAGCCGCCGAAGAGUCUCUGCCUAUCGAAGUGAUGGUCGACGCCGUGCACAAGCUGAAGUACGAGAACUACACCAGCAGCUUUUUCAUCCGGGACAUCAUCAAGCCCGAUCCUCCAAAGAACCUGCAGCUGAAGCCUCUGAAGAACAGCAGACAGGUGGAAGUGUCCUGGGAGUACCCCGACACCUGGUCUACACCCCACAGCUACUUCAGCCUGACCUUUUGCGUGCAAGUGCAGGGCAAGUCCAAGCGCGAGAAAAAGGACCGGGUGUUCACCGACAAGACCAGCGCCACCGUGAUCUGCAGAAAGAACGCCAGCAUCAGCGUCAGAGCCCAGGACCGGUACUACAGCAGCUCUUGGAGCGAAUGGGCCAGCGUGCCAUGUAGCGGAGGUGGUGGUAGCGGAGGCGGCGGAAGCGGCGGUGGUGGAUCAGGUGGUGGUGGCUCUAGAAACCUGCCAGUGGCUACCCCUGAUCCUGGCAUGUUCCCUUGUCUGCACCACAGCCAGAACCUGCUGAGAGCCGUGUCCAACAUGCUGCAGAAGGCCAGACAGACCCUGGAAUUCUACCCCUGCACCAGCGAGGAAAUCGACCACGAGGACAUCACCAAGGAUAAGACCAGCACCGUGGAAGCCUGCCUGCCUCUGGAACUGACCAAGAACGAGAGCUGCCUGAACAGCCGGGAAACCUCCUUCAUCACCAACGGCUCUUGCCUGGCCAGCAGAAAGACAAGCUUCAUGAUGGCCCUGUGCCUGAGCAGCAUCUACGAGGACCUGAAGAUGUACCAGGUGGAAUUCAAGACCAUGAACGCCAAGCUGCUGAUGGACCCCAAGCGGCAGAUCUUCCUGGACCAGAACAUGCUGGCUGUGAUCGACGAGCUGAUGCAGGCCCUGAACUUCAACAGCGAGACAGUGCCCCAGAAGUCUAGCCUGGAAGAACCCGACUUCUACAAGACCAAGAUCAAGCUGUGCAUCCUGCUGCACGCCUUCCGGAUCAGAGCCGUGACCAUCGACAGAGUGAUGAGCUACCUGAACGCCUCCUGAGCUGCCUUCUGCGGGGCUUGCCUUCUGGCCAUGCCCUUCUUCUCUCCCUUGCACCUGUACCUCUUGGUCUUUGAAUAAAGCCUGAGUAGGAAGUCUAGAAAAAAAAAAAAAAAAAAAAAAAAAAAAAAAAAAAAAAAAAAAAAAAAAAAAAAAAAAAAAAAAAAAAAAAAAAAAAAAA